SEQUENCE LISTING (674 sequences)

SEQ ID NO: 1 - DNA, 24 bp, Synthetic construct
  source 1..24: mol_type = other DNA
tggctactatgtattgatgttaac

SEQ ID NO: 2 - DNA, 24 bp, Synthetic construct
  source 1..24: mol_type = other DNA
ttaacatcaatacatagtagccag

SEQ ID NO: 3 - DNA, 24 bp, Synthetic construct
  source 1..24: mol_type = other DNA
ccattaagatgtactcgctctgta

SEQ ID NO: 4 - DNA, 24 bp, Synthetic construct
  source 1..24: mol_type = other DNA
acagagcgagtacatcttaatgga

SEQ ID NO: 5 - DNA, 24 bp, Synthetic construct
  source 1..24: mol_type = other DNA
taatcatttttgtctgttattttt

SEQ ID NO: 6 - DNA, 24 bp, Synthetic construct
  source 1..24: mol_type = other DNA
aaaataacagacaaaaatgattac

SEQ ID NO: 7 - DNA, 24 bp, Synthetic construct
  source 1..24: mol_type = other DNA
gtctatattcagacagatgataat

SEQ ID NO: 8 - DNA, 24 bp, Synthetic construct
  source 1..24: mol_type = other DNA
ttatcatctgtctgaatatagaca

SEQ ID NO: 9 - DNA, 24 bp, Synthetic construct
  source 1..24: mol_type = other DNA
ctagacacatataccaagtaatga

SEQ ID NO: 10 - DNA, 24 bp, Synthetic construct
  source 1..24: mol_type = other DNA
cattacttggtatatgtgtctaga

SEQ ID NO: 11 - DNA, 24 bp, Synthetic construct
  source 1..24: mol_type = other DNA
ggatatagggagtatgaacaactg

SEQ ID NO: 12 - DNA, 24 bp, Synthetic construct
  source 1..24: mol_type = other DNA
agttgttcatactccctatatcct

SEQ ID NO: 13 - DNA, 24 bp, Synthetic construct
  source 1..24: mol_type = other DNA
caattagttccgtattgataatat

SEQ ID NO: 14 - DNA, 24 bp, Synthetic construct
  source 1..24: mol_type = other DNA
tattatcaatacggaactaattgt

SEQ ID NO: 15 - DNA, 24 bp, Synthetic construct
  source 1..24: mol_type = other DNA
ggatcgacgtatttataataatac

SEQ ID NO: 16 - DNA, 24 bp, Synthetic construct
  source 1..24: mol_type = other DNA
tattattataaatacgtcgatcca

SEQ ID NO: 17 - DNA, 24 bp, Synthetic construct
  source 1..24: mol_type = other DNA
tgttctatatctatatttaattat

SEQ ID NO: 18 - DNA, 24 bp, Synthetic construct
  source 1..24: mol_type = other DNA
taattaaatatagatatagaacag

SEQ ID NO: 19 - DNA, 24 bp, Synthetic construct
  source 1..24: mol_type = other DNA
gcggaggtctccactcttctctct

SEQ ID NO: 20 - DNA, 24 bp, Synthetic construct
  source 1..24: mol_type = other DNA
gagagaagagtggagacctccgca

SEQ ID NO: 21 - DNA, 24 bp, Synthetic construct
  source 1..24: mol_type = other DNA
ccatcttatcatcgcccacgtaca

SEQ ID NO: 22 - DNA, 24 bp, Synthetic construct
  source 1..24: mol_type = other DNA
gtacgtgggcgatgataagatgga

SEQ ID NO: 23 - DNA, 24 bp, Synthetic construct
  source 1..24: mol_type = other DNA
cccaattcctcgcaactgggctcc

SEQ ID NO: 24 - DNA, 24 bp, Synthetic construct
  source 1..24: mol_type = other DNA
gagcccagttgcgaggaattgggt

SEQ ID NO: 25 - DNA, 24 bp, Synthetic construct
  source 1..24: mol_type = other DNA
cgcctccacgacactgccccccgc

SEQ ID NO: 26 - DNA, 24 bp, Synthetic construct
  source 1..24: mol_type = other DNA
cggggggcagtgtcgtggaggcgg

SEQ ID NO: 27 - DNA, 24 bp, Synthetic construct
  source 1..24: mol_type = other DNA
caagtccgccgcctccattcttca

SEQ ID NO: 28 - DNA, 24 bp, Synthetic construct
  source 1..24: mol_type = other DNA
gaagaatggaggcggcggacttga

SEQ ID NO: 29 - DNA, 25 bp, Synthetic construct
  source 1..25: mol_type = other DNA
gtgcgttggtgggtctgaaacaata

SEQ ID NO: 30 - DNA, 25 bp, Synthetic construct
  source 1..25: mol_type = other DNA
attgtttcagacccaccaacgcaca

SEQ ID NO: 31 - DNA, 24 bp, Synthetic construct
  source 1..24: mol_type = other DNA
gtctcaaaaggaggtgagctggga

SEQ ID NO: 32 - DNA, 24 bp, Synthetic construct
  source 1..24: mol_type = other DNA
cccagctcacctccttttgagacg

SEQ ID NO: 33 - DNA, 24 bp, Synthetic construct
  source 1..24: mol_type = other DNA
tttagccgactaattttagatgag

SEQ ID NO: 34 - DNA, 24 bp, Synthetic construct
  source 1..24: mol_type = other DNA
tcatctaaaattagtcggctaaat

SEQ ID NO: 35 - DNA, 24 bp, Synthetic construct
  source 1..24: mol_type = other DNA
taatctttagttccgtgcccccgc

SEQ ID NO: 36 - DNA, 24 bp, Synthetic construct
  source 1..24: mol_type = other DNA
cgggggcacggaactaaagattat

SEQ ID NO: 37 - DNA, 24 bp, Synthetic construct
  source 1..24: mol_type = other DNA
tgcaggctcaccccgacgtgcccc

SEQ ID NO: 38 - DNA, 24 bp, Synthetic construct
  source 1..24: mol_type = other DNA
gggcacgtcggggtgagcctgcac

SEQ ID NO: 39 - DNA, 24 bp, Synthetic construct
  source 1..24: mol_type = other DNA
gccctccgcgcgcgatgcccccat

SEQ ID NO: 40 - DNA, 24 bp, Synthetic construct
  source 1..24: mol_type = other DNA
tgggggcatcgcgcgcggagggca

SEQ ID NO: 41 - DNA, 24 bp, Synthetic construct
  source 1..24: mol_type = other DNA
caccgacgcagagctcgcccatgc

SEQ ID NO: 42 - DNA, 24 bp, Synthetic construct
  source 1..24: mol_type = other DNA
catgggcgagctctgcgtcggtgt

SEQ ID NO: 43 - DNA, 24 bp, Synthetic construct
  source 1..24: mol_type = other DNA
cacgtgccccggcggcgtcgcgat

SEQ ID NO: 44 - DNA, 24 bp, Synthetic construct
  source 1..24: mol_type = other DNA
tcgcgacgccgccggggcacgtgc

SEQ ID NO: 45 - DNA, 24 bp, Synthetic construct
  source 1..24: mol_type = other DNA
tatccccgccgtcgcgcgcctacg

SEQ ID NO: 46 - DNA, 24 bp, Synthetic construct
  source 1..24: mol_type = other DNA
gtaggcgcgcgacggcggggatac

SEQ ID NO: 47 - DNA, 24 bp, Synthetic construct
  source 1..24: mol_type = other DNA
gtacctggagcagaagcgatttcg

SEQ ID NO: 48 - DNA, 24 bp, Synthetic construct
  source 1..24: mol_type = other DNA
gaaatcgcttctgctccaggtaca

SEQ ID NO: 49 - DNA, 24 bp, Synthetic construct
  source 1..24: mol_type = other DNA
tgccgaggagctcaccgctgtgaa

SEQ ID NO: 50 - DNA, 24 bp, Synthetic construct
  source 1..24: mol_type = other DNA
tcacagcggtgagctcctcggcaa

SEQ ID NO: 51 - DNA, 24 bp, Synthetic construct
  source 1..24: mol_type = other DNA
tagtcccgtcccctccaggtcctg

SEQ ID NO: 52 - DNA, 24 bp, Synthetic construct
  source 1..24: mol_type = other DNA
aggacctggaggggacgggactaa

SEQ ID NO: 53 - DNA, 24 bp, Synthetic construct
  source 1..24: mol_type = other DNA
aattcacttgtgtattcccccgaa

SEQ ID NO: 54 - DNA, 24 bp, Synthetic construct
  source 1..24: mol_type = other DNA
tcgggggaatacacaagtgaattg

SEQ ID NO: 55 - DNA, 24 bp, Synthetic construct
  source 1..24: mol_type = other DNA
gagacagtctgcacggctcctatc

SEQ ID NO: 56 - DNA, 24 bp, Synthetic construct
  source 1..24: mol_type = other DNA
ataggagccgtgcagactgtctcg

SEQ ID NO: 57 - DNA, 24 bp, Synthetic construct
  source 1..24: mol_type = other DNA
gcatgtacccaataccctgttttg

SEQ ID NO: 58 - DNA, 24 bp, Synthetic construct
  source 1..24: mol_type = other DNA
aaaacagggtattgggtacatgct

SEQ ID NO: 59 - DNA, 24 bp, Synthetic construct
  source 1..24: mol_type = other DNA
tgaccttgggtactattatagcac

SEQ ID NO: 60 - DNA, 24 bp, Synthetic construct
  source 1..24: mol_type = other DNA
tgctataatagtacccaaggtcaa

SEQ ID NO: 61 - DNA, 24 bp, Synthetic construct
  source 1..24: mol_type = other DNA
tttcagttcgagtgttagaccctg

SEQ ID NO: 62 - DNA, 24 bp, Synthetic construct
  source 1..24: mol_type = other DNA
agggtctaacactcgaactgaaac

SEQ ID NO: 63 - DNA, 24 bp, Synthetic construct
  source 1..24: mol_type = other DNA
ctttttggaattcttctgaccagg

SEQ ID NO: 64 - DNA, 24 bp, Synthetic construct
  source 1..24: mol_type = other DNA
ctggtcagaagaattccaaaaaga

SEQ ID NO: 65 - DNA, 24 bp, Synthetic construct
  source 1..24: mol_type = other DNA
caagaaaattaaggatatatagcg

SEQ ID NO: 66 - DNA, 24 bp, Synthetic construct
  source 1..24: mol_type = other DNA
gctatatatccttaattttcttgg

SEQ ID NO: 67 - DNA, 24 bp, Synthetic construct
  source 1..24: mol_type = other DNA
taaggcatgtcgataataaagact

SEQ ID NO: 68 - DNA, 24 bp, Synthetic construct
  source 1..24: mol_type = other DNA
gtctttattatcgacatgccttag

SEQ ID NO: 69 - DNA, 24 bp, Synthetic construct
  source 1..24: mol_type = other DNA
ataggaccgagcaactcggattcg

SEQ ID NO: 70 - DNA, 24 bp, Synthetic construct
  source 1..24: mol_type = other DNA
gaatccgagttgctcggtcctatt

SEQ ID NO: 71 - DNA, 24 bp, Synthetic construct
  source 1..24: mol_type = other DNA
gactcggacttggactgtgcaact

SEQ ID NO: 72 - DNA, 24 bp, Synthetic construct
  source 1..24: mol_type = other DNA
gttgcacagtccaagtccgagtca

SEQ ID NO: 73 - DNA, 24 bp, Synthetic construct
  source 1..24: mol_type = other DNA
agccgggatactcaaaatcgaaat

SEQ ID NO: 74 - DNA, 24 bp, Synthetic construct
  source 1..24: mol_type = other DNA
tttcgattttgagtatcccggctg

SEQ ID NO: 75 - DNA, 24 bp, Synthetic construct
  source 1..24: mol_type = other DNA
actcggtccataggttaatactca

SEQ ID NO: 76 - DNA, 24 bp, Synthetic construct
  source 1..24: mol_type = other DNA
gagtattaacctatggaccgagtc

SEQ ID NO: 77 - DNA, 24 bp, Synthetic construct
  source 1..24: mol_type = other DNA
tcatattttacatctaaacgttcg

SEQ ID NO: 78 - DNA, 24 bp, Synthetic construct
  source 1..24: mol_type = other DNA
gaacgtttagatgtaaaatatgag

SEQ ID NO: 79 - DNA, 24 bp, Synthetic construct
  source 1..24: mol_type = other DNA
tggaagcaaacacccccttagacg

SEQ ID NO: 80 - DNA, 24 bp, Synthetic construct
  source 1..24: mol_type = other DNA
gtctaagggggtgtttgcttccac

SEQ ID NO: 81 - DNA, 24 bp, Synthetic construct
  source 1..24: mol_type = other DNA
ccacctcttgtcgaccatttgtag

SEQ ID NO: 82 - DNA, 24 bp, Synthetic construct
  source 1..24: mol_type = other DNA
tacaaatggtcgacaagaggtggt

SEQ ID NO: 83 - DNA, 24 bp, Synthetic construct
  source 1..24: mol_type = other DNA
catgttccgttctgccgactgatg

SEQ ID NO: 84 - DNA, 24 bp, Synthetic construct
  source 1..24: mol_type = other DNA
atcagtcggcagaacggaacatga

SEQ ID NO: 85 - DNA, 24 bp, Synthetic construct
  source 1..24: mol_type = other DNA
cctgcagctgcagctgcagtgcgt

SEQ ID NO: 86 - DNA, 24 bp, Synthetic construct
  source 1..24: mol_type = other DNA
cgcactgcagctgcagctgcagga

SEQ ID NO: 87 - DNA, 24 bp, Synthetic construct
  source 1..24: mol_type = other DNA
actccatcgtctattaatggctcg

SEQ ID NO: 88 - DNA, 24 bp, Synthetic construct
  source 1..24: mol_type = other DNA
gagccattaatagacgatggagta

SEQ ID NO: 89 - DNA, 24 bp, Synthetic construct
  source 1..24: mol_type = other DNA
ccagcgctcggcttagacaagcct

SEQ ID NO: 90 - DNA, 24 bp, Synthetic construct
  source 1..24: mol_type = other DNA
ggcttgtctaagccgagcgctgga

SEQ ID NO: 91 - DNA, 24 bp, Synthetic construct
  source 1..24: mol_type = other DNA
atgaaggtcagtcagtagtcccac

SEQ ID NO: 92 - DNA, 24 bp, Synthetic construct
  source 1..24: mol_type = other DNA
tgggactactgactgaccttcatg

SEQ ID NO: 93 - DNA, 24 bp, Synthetic construct
  source 1..24: mol_type = other DNA
agcttttagtctagctcgacagtc

SEQ ID NO: 94 - DNA, 24 bp, Synthetic construct
  source 1..24: mol_type = other DNA
actgtcgagctagactaaaagctg

SEQ ID NO: 95 - DNA, 24 bp, Synthetic construct
  source 1..24: mol_type = other DNA
ttctttctaatccacctattttct

SEQ ID NO: 96 - DNA, 24 bp, Synthetic construct
  source 1..24: mol_type = other DNA
gaaaataggtggattagaaagaac

SEQ ID NO: 97 - DNA, 24 bp, Synthetic construct
  source 1..24: mol_type = other DNA
gcgactcactctcgtagttggtgt

SEQ ID NO: 98 - DNA, 24 bp, Synthetic construct
  source 1..24: mol_type = other DNA
caccaactacgagagtgagtcgcc

SEQ ID NO: 99 - DNA, 24 bp, Synthetic construct
  source 1..24: mol_type = other DNA
tcatgacctcctcctcaagctcgg

SEQ ID NO: 100 - DNA, 24 bp, Synthetic construct
  source 1..24: mol_type = other DNA
cgagcttgaggaggaggtcatgaa

SEQ ID NO: 101 - DNA, 24 bp, Synthetic construct
  source 1..24: mol_type = other DNA
ctcatatttgaagcctccttgttc

SEQ ID NO: 102 - DNA, 24 bp, Synthetic construct
  source 1..24: mol_type = other DNA
aacaaggaggcttcaaatatgaga

SEQ ID NO: 103 - DNA, 24 bp, Synthetic construct
  source 1..24: mol_type = other DNA
agaaagcatcgaccttagcaaggt

SEQ ID NO: 104 - DNA, 24 bp, Synthetic construct
  source 1..24: mol_type = other DNA
ccttgctaaggtcgatgctttctt

SEQ ID NO: 105 - DNA, 24 bp, Synthetic construct
  source 1..24: mol_type = other DNA
ttgtccatgcgcttggtgaggtcg

SEQ ID NO: 106 - DNA, 24 bp, Synthetic construct
  source 1..24: mol_type = other DNA
gacctcaccaagcgcatggacaag

SEQ ID NO: 107 - DNA, 24 bp, Synthetic construct
  source 1..24: mol_type = other DNA
aatctaacctttgagtaccaaatg

SEQ ID NO: 108 - DNA, 24 bp, Synthetic construct
  source 1..24: mol_type = other DNA
atttggtactcaaaggttagattg

SEQ ID NO: 109 - DNA, 24 bp, Synthetic construct
  source 1..24: mol_type = other DNA
tggagacacatacacagagaagaa

SEQ ID NO: 110 - DNA, 24 bp, Synthetic construct
  source 1..24: mol_type = other DNA
tcttctctgtgtatgtgtctccat

SEQ ID NO: 111 - DNA, 24 bp, Synthetic construct
  source 1..24: mol_type = other DNA
cttgtttggattgaaaccattaca

SEQ ID NO: 112 - DNA, 24 bp, Synthetic construct
  source 1..24: mol_type = other DNA
gtaatggtttcaatccaaacaaga

SEQ ID NO: 113 - DNA, 24 bp, Synthetic construct
  source 1..24: mol_type = other DNA
atatattggacttgtattccaagc

SEQ ID NO: 114 - DNA, 24 bp, Synthetic construct
  source 1..24: mol_type = other DNA
cttggaatacaagtccaatatata

SEQ ID NO: 115 - DNA, 24 bp, Synthetic construct
  source 1..24: mol_type = other DNA
gtccttatagatttggacacttat

SEQ ID NO: 116 - DNA, 24 bp, Synthetic construct
  source 1..24: mol_type = other DNA
taagtgtccaaatctataaggact

SEQ ID NO: 117 - DNA, 24 bp, Synthetic construct
  source 1..24: mol_type = other DNA
caaatcttcttgcctaagcaaatt

SEQ ID NO: 118 - DNA, 24 bp, Synthetic construct
  source 1..24: mol_type = other DNA
atttgcttaggcaagaagatttgt

SEQ ID NO: 119 - DNA, 24 bp, Synthetic construct
  source 1..24: mol_type = other DNA
ctaaactctattttatactccctc

SEQ ID NO: 120 - DNA, 24 bp, Synthetic construct
  source 1..24: mol_type = other DNA
agggagtataaaatagagtttagt

SEQ ID NO: 121 - DNA, 24 bp, Synthetic construct
  source 1..24: mol_type = other DNA
agtgttcattttggctcctcattt

SEQ ID NO: 122 - DNA, 24 bp, Synthetic construct
  source 1..24: mol_type = other DNA
aatgaggagccaaaatgaacacta

SEQ ID NO: 123 - DNA, 24 bp, Synthetic construct
  source 1..24: mol_type = other DNA
tcagatggatgaaaatgaatctag

SEQ ID NO: 124 - DNA, 24 bp, Synthetic construct
  source 1..24: mol_type = other DNA
tagattcattttcatccatctgaa

SEQ ID NO: 125 - DNA, 24 bp, Synthetic construct
  source 1..24: mol_type = other DNA
tgaatccactgatatgttaaaacg

SEQ ID NO: 126 - DNA, 24 bp, Synthetic construct
  source 1..24: mol_type = other DNA
gttttaacatatcagtggattcat

SEQ ID NO: 127 - DNA, 24 bp, Synthetic construct
  source 1..24: mol_type = other DNA
gggacggagagagtatattccaag

SEQ ID NO: 128 - DNA, 24 bp, Synthetic construct
  source 1..24: mol_type = other DNA
ttggaatatactctctccgtccca

SEQ ID NO: 129 - DNA, 24 bp, Synthetic construct
  source 1..24: mol_type = other DNA
ctatctttgggttttcatcttttt

SEQ ID NO: 130 - DNA, 24 bp, Synthetic construct
  source 1..24: mol_type = other DNA
aaaagatgaaaacccaaagatagc

SEQ ID NO: 131 - DNA, 24 bp, Synthetic construct
  source 1..24: mol_type = other DNA
gaccaggagggactctatttatat

SEQ ID NO: 132 - DNA, 24 bp, Synthetic construct
  source 1..24: mol_type = other DNA
tataaatagagtccctcctggtca

SEQ ID NO: 133 - DNA, 24 bp, Synthetic construct
  source 1..24: mol_type = other DNA
gataataaagactctgactaatag

SEQ ID NO: 134 - DNA, 24 bp, Synthetic construct
  source 1..24: mol_type = other DNA
tattagtcagagtctttattatcg

SEQ ID NO: 135 - DNA, 24 bp, Synthetic construct
  source 1..24: mol_type = other DNA
agactctgactaataggaccgagc

SEQ ID NO: 136 - DNA, 24 bp, Synthetic construct
  source 1..24: mol_type = other DNA
ctcggtcctattagtcagagtctt

SEQ ID NO: 137 - DNA, 24 bp, Synthetic construct
  source 1..24: mol_type = other DNA
actaataggaccgagcaactcgga

SEQ ID NO: 138 - DNA, 24 bp, Synthetic construct
  source 1..24: mol_type = other DNA
ccgagttgctcggtcctattagtc

SEQ ID NO: 139 - DNA, 24 bp, Synthetic construct
  source 1..24: mol_type = other DNA
gaccgagcaactcggattcggtgg

SEQ ID NO: 140 - DNA, 24 bp, Synthetic construct
  source 1..24: mol_type = other DNA
caccgaatccgagttgctcggtcc

SEQ ID NO: 141 - DNA, 24 bp, Synthetic construct
  source 1..24: mol_type = other DNA
aactcggattcggtggagtgactc

SEQ ID NO: 142 - DNA, 24 bp, Synthetic construct
  source 1..24: mol_type = other DNA
agtcactccaccgaatccgagttg

SEQ ID NO: 143 - DNA, 24 bp, Synthetic construct
  source 1..24: mol_type = other DNA
ttcggtggagtgactcggacttgg

SEQ ID NO: 144 - DNA, 24 bp, Synthetic construct
  source 1..24: mol_type = other DNA
caagtccgagtcactccaccgaat

SEQ ID NO: 145 - DNA, 24 bp, Synthetic construct
  source 1..24: mol_type = other DNA
agtgactcggacttggactgtgca

SEQ ID NO: 146 - DNA, 24 bp, Synthetic construct
  source 1..24: mol_type = other DNA
gcacagtccaagtccgagtcactc

SEQ ID NO: 147 - DNA, 24 bp, Synthetic construct
  source 1..24: mol_type = other DNA
ggacttggactgtgcaactcggat

SEQ ID NO: 148 - DNA, 24 bp, Synthetic construct
  source 1..24: mol_type = other DNA
tccgagttgcacagtccaagtccg

SEQ ID NO: 149 - DNA, 24 bp, Synthetic construct
  source 1..24: mol_type = other DNA
actgtgcaactcggattcagccgg

SEQ ID NO: 150 - DNA, 24 bp, Synthetic construct
  source 1..24: mol_type = other DNA
cggctgaatccgagttgcacagtc

SEQ ID NO: 151 - DNA, 24 bp, Synthetic construct
  source 1..24: mol_type = other DNA
actcggattcagccgggatactca

SEQ ID NO: 152 - DNA, 24 bp, Synthetic construct
  source 1..24: mol_type = other DNA
gagtatcccggctgaatccgagtt

SEQ ID NO: 153 - DNA, 24 bp, Synthetic construct
  source 1..24: mol_type = other DNA
tcagccgggatactcaaaatcgaa

SEQ ID NO: 154 - DNA, 24 bp, Synthetic construct
  source 1..24: mol_type = other DNA
tcgattttgagtatcccggctgaa

SEQ ID NO: 155 - DNA, 24 bp, Synthetic construct
  source 1..24: mol_type = other DNA
gatactcaaaatcgaaatccaagg

SEQ ID NO: 156 - DNA, 24 bp, Synthetic construct
  source 1..24: mol_type = other DNA
cttggatttcgattttgagtatcc

SEQ ID NO: 157 - DNA, 24 bp, Synthetic construct
  source 1..24: mol_type = other DNA
aaatcgaaatccaagggactcggt

SEQ ID NO: 158 - DNA, 24 bp, Synthetic construct
  source 1..24: mol_type = other DNA
ccgagtcccttggatttcgatttt

SEQ ID NO: 159 - DNA, 24 bp, Synthetic construct
  source 1..24: mol_type = other DNA
atccaagggactcggtccataggt

SEQ ID NO: 160 - DNA, 24 bp, Synthetic construct
  source 1..24: mol_type = other DNA
cctatggaccgagtcccttggatt

SEQ ID NO: 161 - DNA, 24 bp, Synthetic construct
  source 1..24: mol_type = other DNA
gactcggtccataggttaatactc

SEQ ID NO: 162 - DNA, 24 bp, Synthetic construct
  source 1..24: mol_type = other DNA
agtattaacctatggaccgagtcc

SEQ ID NO: 163 - DNA, 24 bp, Synthetic construct
  source 1..24: mol_type = other DNA
tcatattttacatctaaacgttcg

SEQ ID NO: 164 - DNA, 24 bp, Synthetic construct
  source 1..24: mol_type = other DNA
gaacgtttagatgtaaaatatgag

SEQ ID NO: 165 - DNA, 24 bp, Synthetic construct
  source 1..24: mol_type = other DNA
tacatctaaacgttcgatgtgcgt

SEQ ID NO: 166 - DNA, 24 bp, Synthetic construct
  source 1..24: mol_type = other DNA
cgcacatcgaacgtttagatgtaa

SEQ ID NO: 167 - DNA, 24 bp, Synthetic construct
  source 1..24: mol_type = other DNA
aacgttcgatgtgcgtggaagcaa

SEQ ID NO: 168 - DNA, 24 bp, Synthetic construct
  source 1..24: mol_type = other DNA
tgcttccacgcacatcgaacgttt

SEQ ID NO: 169 - DNA, 24 bp, Synthetic construct
  source 1..24: mol_type = other DNA
atgtgcgtggaagcaaacaccccc

SEQ ID NO: 170 - DNA, 24 bp, Synthetic construct
  source 1..24: mol_type = other DNA
ggggtgtttgcttccacgcacatc

SEQ ID NO: 171 - DNA, 24 bp, Synthetic construct
  source 1..24: mol_type = other DNA
ggaagcaaacacccccttagacgt

SEQ ID NO: 172 - DNA, 24 bp, Synthetic construct
  source 1..24: mol_type = other DNA
cgtctaagggggtgtttgcttcca

SEQ ID NO: 173 - DNA, 24 bp, Synthetic construct
  source 1..24: mol_type = other DNA
acacccccttagacgtgggacacc

SEQ ID NO: 174 - DNA, 24 bp, Synthetic construct
  source 1..24: mol_type = other DNA
gtgtcccacgtctaagggggtgtt

SEQ ID NO: 175 - DNA, 24 bp, Synthetic construct
  source 1..24: mol_type = other DNA
ttagacgtgggacaccacctcttg

SEQ ID NO: 176 - DNA, 24 bp, Synthetic construct
  source 1..24: mol_type = other DNA
aagaggtggtgtcccacgtctaag

SEQ ID NO: 177 - DNA, 24 bp, Synthetic construct
  source 1..24: mol_type = other DNA
gggacaccacctcttgtcgaccat

SEQ ID NO: 178 - DNA, 24 bp, Synthetic construct
  source 1..24: mol_type = other DNA
tggtcgacaagaggtggtgtccca

SEQ ID NO: 179 - DNA, 24 bp, Synthetic construct
  source 1..24: mol_type = other DNA
acctcttgtcgaccatttgtagcc

SEQ ID NO: 180 - DNA, 24 bp, Synthetic construct
  source 1..24: mol_type = other DNA
gctacaaatggtcgacaagaggtg

SEQ ID NO: 181 - DNA, 24 bp, Synthetic construct
  source 1..24: mol_type = other DNA
tcgaccatttgtagccttcttcat

SEQ ID NO: 182 - DNA, 24 bp, Synthetic construct
  source 1..24: mol_type = other DNA
tgaagaaggctacaaatggtcgac

SEQ ID NO: 183 - DNA, 24 bp, Synthetic construct
  source 1..24: mol_type = other DNA
ttgtagccttcttcatgttccgtt

SEQ ID NO: 184 - DNA, 24 bp, Synthetic construct
  source 1..24: mol_type = other DNA
acggaacatgaagaaggctacaaa

SEQ ID NO: 185 - DNA, 24 bp, Synthetic construct
  source 1..24: mol_type = other DNA
ttcttcatgttccgttctgccgac

SEQ ID NO: 186 - DNA, 24 bp, Synthetic construct
  source 1..24: mol_type = other DNA
tcggcagaacggaacatgaagaag

SEQ ID NO: 187 - DNA, 24 bp, Synthetic construct
  source 1..24: mol_type = other DNA
gttccgttctgccgactgatggat

SEQ ID NO: 188 - DNA, 24 bp, Synthetic construct
  source 1..24: mol_type = other DNA
tccatcagtcggcagaacggaaca

SEQ ID NO: 189 - DNA, 24 bp, Synthetic construct
  source 1..24: mol_type = other DNA
ctgccgactgatggatcactcctg

SEQ ID NO: 190 - DNA, 24 bp, Synthetic construct
  source 1..24: mol_type = other DNA
aggagtgatccatcagtcggcaga

SEQ ID NO: 191 - DNA, 24 bp, Synthetic construct
  source 1..24: mol_type = other DNA
tgatggatcactcctgcagctgca

SEQ ID NO: 192 - DNA, 24 bp, Synthetic construct
  source 1..24: mol_type = other DNA
gcagctgcaggagtgatccatcag

SEQ ID NO: 193 - DNA, 24 bp, Synthetic construct
  source 1..24: mol_type = other DNA
cactcctgcagctgcagctgcagt

SEQ ID NO: 194 - DNA, 24 bp, Synthetic construct
  source 1..24: mol_type = other DNA
ctgcagctgcagctgcaggagtga

SEQ ID NO: 195 - DNA, 24 bp, Synthetic construct
  source 1..24: mol_type = other DNA
atgaagcaaaactctaaagttgac

SEQ ID NO: 196 - DNA, 24 bp, Synthetic construct
  source 1..24: mol_type = other DNA
tcaactttagagttttgcttcata

SEQ ID NO: 197 - DNA, 24 bp, Synthetic construct
  source 1..24: mol_type = other DNA
aacccagtttttcagctctcacta

SEQ ID NO: 198 - DNA, 24 bp, Synthetic construct
  source 1..24: mol_type = other DNA
agtgagagctgaaaaactgggttg

SEQ ID NO: 199 - DNA, 24 bp, Synthetic construct
  source 1..24: mol_type = other DNA
attaccaattgatcatcatcttgc

SEQ ID NO: 200 - DNA, 24 bp, Synthetic construct
  source 1..24: mol_type = other DNA
caagatgatgatcaattggtaatt

SEQ ID NO: 201 - DNA, 24 bp, Synthetic construct
  source 1..24: mol_type = other DNA
attacaactttccagctattttgc

SEQ ID NO: 202 - DNA, 24 bp, Synthetic construct
  source 1..24: mol_type = other DNA
caaaatagctggaaagttgtaata

SEQ ID NO: 203 - DNA, 24 bp, Synthetic construct
  source 1..24: mol_type = other DNA
cactttataatcctaatcctacac

SEQ ID NO: 204 - DNA, 24 bp, Synthetic construct
  source 1..24: mol_type = other DNA
tgtaggattaggattataaagtgt

SEQ ID NO: 205 - DNA, 24 bp, Synthetic construct
  source 1..24: mol_type = other DNA
aagctcattcaacaacacaattag

SEQ ID NO: 206 - DNA, 24 bp, Synthetic construct
  source 1..24: mol_type = other DNA
taattgtgttgttgaatgagcttt

SEQ ID NO: 207 - DNA, 24 bp, Synthetic construct
  source 1..24: mol_type = other DNA
tcaaacacaaaatagagttatggc

SEQ ID NO: 208 - DNA, 24 bp, Synthetic construct
  source 1..24: mol_type = other DNA
ccataactctattttgtgtttgat

SEQ ID NO: 209 - DNA, 24 bp, Synthetic construct
  source 1..24: mol_type = other DNA
ttaacttcatgtactaccatttcc

SEQ ID NO: 210 - DNA, 24 bp, Synthetic construct
  source 1..24: mol_type = other DNA
gaaatggtagtacatgaagttaaa

SEQ ID NO: 211 - DNA, 24 bp, Synthetic construct
  source 1..24: mol_type = other DNA
catccaaaattttcgtccgtccaa

SEQ ID NO: 212 - DNA, 24 bp, Synthetic construct
  source 1..24: mol_type = other DNA
tggacggacgaaaattttggatgg

SEQ ID NO: 213 - DNA, 24 bp, Synthetic construct
  source 1..24: mol_type = other DNA
cgataactttaaggtgaattgtga

SEQ ID NO: 214 - DNA, 24 bp, Synthetic construct
  source 1..24: mol_type = other DNA
cacaattcaccttaaagttatcga

SEQ ID NO: 215 - DNA, 24 bp, Synthetic construct
  source 1..24: mol_type = other DNA
taatgaaggaaaatcttttccagg

SEQ ID NO: 216 - DNA, 24 bp, Synthetic construct
  source 1..24: mol_type = other DNA
ctggaaaagattttccttcattat

SEQ ID NO: 217 - DNA, 24 bp, Synthetic construct
  source 1..24: mol_type = other DNA
gatagacgaaatgtcctccttggt

SEQ ID NO: 218 - DNA, 24 bp, Synthetic construct
  source 1..24: mol_type = other DNA
ccaaggaggacatttcgtctatca

SEQ ID NO: 219 - DNA, 24 bp, Synthetic construct
  source 1..24: mol_type = other DNA
ggctctatggagcatctaatctta

SEQ ID NO: 220 - DNA, 24 bp, Synthetic construct
  source 1..24: mol_type = other DNA
aagattagatgctccatagagccc

SEQ ID NO: 221 - DNA, 24 bp, Synthetic construct
  source 1..24: mol_type = other DNA
aaccaacgagccatttgccctagg

SEQ ID NO: 222 - DNA, 24 bp, Synthetic construct
  source 1..24: mol_type = other DNA
ctagggcaaatggctcgttggtta

SEQ ID NO: 223 - DNA, 24 bp, Synthetic construct
  source 1..24: mol_type = other DNA
gtaccacccccagacttctcaaca

SEQ ID NO: 224 - DNA, 24 bp, Synthetic construct
  source 1..24: mol_type = other DNA
gttgagaagtctgggggtggtacc

SEQ ID NO: 225 - DNA, 24 bp, Synthetic construct
  source 1..24: mol_type = other DNA
tctgtcaaccgacccaaccgaccc

SEQ ID NO: 226 - DNA, 24 bp, Synthetic construct
  source 1..24: mol_type = other DNA
ggtcggttgggtcggttgacagaa

SEQ ID NO: 227 - DNA, 24 bp, Synthetic construct
  source 1..24: mol_type = other DNA
tccagactgtaagttatttttctg

SEQ ID NO: 228 - DNA, 24 bp, Synthetic construct
  source 1..24: mol_type = other DNA
agaaaaataacttacagtctggat

SEQ ID NO: 229 - DNA, 24 bp, Synthetic construct
  source 1..24: mol_type = other DNA
cagagcttctaagaacaaaaactt

SEQ ID NO: 230 - DNA, 24 bp, Synthetic construct
  source 1..24: mol_type = other DNA
agtttttgttcttagaagctctgt

SEQ ID NO: 231 - DNA, 24 bp, Synthetic construct
  source 1..24: mol_type = other DNA
gtatggctgctatacaaaattccc

SEQ ID NO: 232 - DNA, 24 bp, Synthetic construct
  source 1..24: mol_type = other DNA
ggaattttgtatagcagccataca

SEQ ID NO: 233 - DNA, 24 bp, Synthetic construct
  source 1..24: mol_type = other DNA
cgcttcctggaataattgatatgg

SEQ ID NO: 234 - DNA, 24 bp, Synthetic construct
  source 1..24: mol_type = other DNA
catatcaattattccaggaagcgg

SEQ ID NO: 235 - DNA, 24 bp, Synthetic construct
  source 1..24: mol_type = other DNA
tattatataaggcaaggtatagcc

SEQ ID NO: 236 - DNA, 24 bp, Synthetic construct
  source 1..24: mol_type = other DNA
gctataccttgccttatataataa

SEQ ID NO: 237 - DNA, 24 bp, Synthetic construct
  source 1..24: mol_type = other DNA
tcattcaaaacctagcaataatgg

SEQ ID NO: 238 - DNA, 24 bp, Synthetic construct
  source 1..24: mol_type = other DNA
cattattgctaggttttgaatgaa

SEQ ID NO: 239 - DNA, 24 bp, Synthetic construct
  source 1..24: mol_type = other DNA
gtagtaatacatctctcaaaactc

SEQ ID NO: 240 - DNA, 24 bp, Synthetic construct
  source 1..24: mol_type = other DNA
agttttgagagatgtattactact

SEQ ID NO: 241 - DNA, 24 bp, Synthetic construct
  source 1..24: mol_type = other DNA
ttcttcctccacttctttatcttc

SEQ ID NO: 242 - DNA, 24 bp, Synthetic construct
  source 1..24: mol_type = other DNA
aagataaagaagtggaggaagaag

SEQ ID NO: 243 - DNA, 24 bp, Synthetic construct
  source 1..24: mol_type = other DNA
aagccctcttcaacttttcatcca

SEQ ID NO: 244 - DNA, 24 bp, Synthetic construct
  source 1..24: mol_type = other DNA
ggatgaaaagttgaagagggctta

SEQ ID NO: 245 - DNA, 24 bp, Synthetic construct
  source 1..24: mol_type = other DNA
cgtaccaaatgttcaaagtttcat

SEQ ID NO: 246 - DNA, 24 bp, Synthetic construct
  source 1..24: mol_type = other DNA
tgaaactttgaacatttggtacgt

SEQ ID NO: 247 - DNA, 24 bp, Synthetic construct
  source 1..24: mol_type = other DNA
taccaataataacggtgaccaaaa

SEQ ID NO: 248 - DNA, 24 bp, Synthetic construct
  source 1..24: mol_type = other DNA
tttggtcaccgttattattggtaa

SEQ ID NO: 249 - DNA, 24 bp, Synthetic construct
  source 1..24: mol_type = other DNA
gttgaaacgaattctgttgatcga

SEQ ID NO: 250 - DNA, 24 bp, Synthetic construct
  source 1..24: mol_type = other DNA
cgatcaacagaattcgtttcaacg

SEQ ID NO: 251 - DNA, 24 bp, Synthetic construct
  source 1..24: mol_type = other DNA
ttcttcttggcttaggtggtcttt

SEQ ID NO: 252 - DNA, 24 bp, Synthetic construct
  source 1..24: mol_type = other DNA
aagaccacctaagccaagaagaac

SEQ ID NO: 253 - DNA, 24 bp, Synthetic construct
  source 1..24: mol_type = other DNA
tgctaatgctataccattagctgc

SEQ ID NO: 254 - DNA, 24 bp, Synthetic construct
  source 1..24: mol_type = other DNA
cagctaatggtatagcattagcaa

SEQ ID NO: 255 - DNA, 24 bp, Synthetic construct
  source 1..24: mol_type = other DNA
aattatcaatgcttgtgaatctcg

SEQ ID NO: 256 - DNA, 24 bp, Synthetic construct
  source 1..24: mol_type = other DNA
gagattcacaagcattgataattt

SEQ ID NO: 257 - DNA, 24 bp, Synthetic construct
  source 1..24: mol_type = other DNA
tgttaaaaaatttcctcacctacc

SEQ ID NO: 258 - DNA, 24 bp, Synthetic construct
  source 1..24: mol_type = other DNA
gtaggtgaggaaattttttaacaa

SEQ ID NO: 259 - DNA, 24 bp, Synthetic construct
  source 1..24: mol_type = other DNA
gaattgttcgatatgagatcgagc

SEQ ID NO: 260 - DNA, 24 bp, Synthetic construct
  source 1..24: mol_type = other DNA
ctcgatctcatatcgaacaattcg

SEQ ID NO: 261 - DNA, 24 bp, Synthetic construct
  source 1..24: mol_type = other DNA
tggagtaatattttatttggctcc

SEQ ID NO: 262 - DNA, 24 bp, Synthetic construct
  source 1..24: mol_type = other DNA
gagccaaataaaatattactccat

SEQ ID NO: 263 - DNA, 24 bp, Synthetic construct
  source 1..24: mol_type = other DNA
tatataaggcaatgtatagcccta

SEQ ID NO: 264 - DNA, 24 bp, Synthetic construct
  source 1..24: mol_type = other DNA
agggctatacattgccttatataa

SEQ ID NO: 265 - DNA, 24 bp, Synthetic construct
  source 1..24: mol_type = other DNA
tcatccaaaaactagcaatggcaa

SEQ ID NO: 266 - DNA, 24 bp, Synthetic construct
  source 1..24: mol_type = other DNA
tgccattgctagtttttggatgaa

SEQ ID NO: 267 - DNA, 24 bp, Synthetic construct
  source 1..24: mol_type = other DNA
taatagtagtagtaccactctcaa

SEQ ID NO: 268 - DNA, 24 bp, Synthetic construct
  source 1..24: mol_type = other DNA
tgagagtggtactactactattac

SEQ ID NO: 269 - DNA, 24 bp, Synthetic construct
  source 1..24: mol_type = other DNA
tttacttcttcctccacttcttta

SEQ ID NO: 270 - DNA, 24 bp, Synthetic construct
  source 1..24: mol_type = other DNA
aaagaagtggaggaagaagtaaaa

SEQ ID NO: 271 - DNA, 24 bp, Synthetic construct
  source 1..24: mol_type = other DNA
ctcctaagccctctcaacttttcc

SEQ ID NO: 272 - DNA, 24 bp, Synthetic construct
  source 1..24: mol_type = other DNA
gaaaagttgagagggcttaggagt

SEQ ID NO: 273 - DNA, 24 bp, Synthetic construct
  source 1..24: mol_type = other DNA
acgtaacaaaacgttcaaagtttc

SEQ ID NO: 274 - DNA, 24 bp, Synthetic construct
  source 1..24: mol_type = other DNA
aaactttgaacgttttgttacgtt

SEQ ID NO: 275 - DNA, 24 bp, Synthetic construct
  source 1..24: mol_type = other DNA
gttaccaataataacggtgaccaa

SEQ ID NO: 276 - DNA, 24 bp, Synthetic construct
  source 1..24: mol_type = other DNA
tggtcaccgttattattggtaacc

SEQ ID NO: 277 - DNA, 24 bp, Synthetic construct
  source 1..24: mol_type = other DNA
acgttgaaacgaattctgttgatc

SEQ ID NO: 278 - DNA, 24 bp, Synthetic construct
  source 1..24: mol_type = other DNA
atcaacagaattcgtttcaacgtt

SEQ ID NO: 279 - DNA, 24 bp, Synthetic construct
  source 1..24: mol_type = other DNA
tgttcttcttggtttaggaggtct

SEQ ID NO: 280 - DNA, 24 bp, Synthetic construct
  source 1..24: mol_type = other DNA
gacctcctaaaccaagaagaacat

SEQ ID NO: 281 - DNA, 24 bp, Synthetic construct
  source 1..24: mol_type = other DNA
gttgctaatgctataccattagct

SEQ ID NO: 282 - DNA, 24 bp, Synthetic construct
  source 1..24: mol_type = other DNA
gctaatggtatagcattagcaaca

SEQ ID NO: 283 - DNA, 24 bp, Synthetic construct
  source 1..24: mol_type = other DNA
cttctccaactccacctcctgatc

SEQ ID NO: 284 - DNA, 24 bp, Synthetic construct
  source 1..24: mol_type = other DNA
atcaggaggtggagttggagaagc

SEQ ID NO: 285 - DNA, 24 bp, Synthetic construct
  source 1..24: mol_type = other DNA
ccaaacaatctgttcagctattca

SEQ ID NO: 286 - DNA, 24 bp, Synthetic construct
  source 1..24: mol_type = other DNA
gaatagctgaacagattgtttggt

SEQ ID NO: 287 - DNA, 24 bp, Synthetic construct
  source 1..24: mol_type = other DNA
aaatgtgtaaaagatttcccacac

SEQ ID NO: 288 - DNA, 24 bp, Synthetic construct
  source 1..24: mol_type = other DNA
tgtgggaaatcttttacacatttt

SEQ ID NO: 289 - DNA, 24 bp, Synthetic construct
  source 1..24: mol_type = other DNA
tcaaaaacctcccacctaccgcgt

SEQ ID NO: 290 - DNA, 24 bp, Synthetic construct
  source 1..24: mol_type = other DNA
cgcggtaggtgggaggtttttgaa

SEQ ID NO: 291 - DNA, 24 bp, Synthetic construct
  source 1..24: mol_type = other DNA
tgttggagtggtaggtgagcctct

SEQ ID NO: 292 - DNA, 24 bp, Synthetic construct
  source 1..24: mol_type = other DNA
gaggctcacctaccactccaacat

SEQ ID NO: 293 - DNA, 24 bp, Synthetic construct
  source 1..24: mol_type = other DNA
agcaacatactatataatgcaagg

SEQ ID NO: 294 - DNA, 24 bp, Synthetic construct
  source 1..24: mol_type = other DNA
cttgcattatatagtatgttgcta

SEQ ID NO: 295 - DNA, 24 bp, Synthetic construct
  source 1..24: mol_type = other DNA
tatgaatcttcatcaaccaaaagc

SEQ ID NO: 296 - DNA, 24 bp, Synthetic construct
  source 1..24: mol_type = other DNA
cttttggttgatgaagattcatat

SEQ ID NO: 297 - DNA, 24 bp, Synthetic construct
  source 1..24: mol_type = other DNA
aatggcaagtgtgtgcaatagtag

SEQ ID NO: 298 - DNA, 24 bp, Synthetic construct
  source 1..24: mol_type = other DNA
tactattgcacacacttgccattg

SEQ ID NO: 299 - DNA, 24 bp, Synthetic construct
  source 1..24: mol_type = other DNA
actacaactctcaaaactcctttc

SEQ ID NO: 300 - DNA, 24 bp, Synthetic construct
  source 1..24: mol_type = other DNA
aaaggagttttgagagttgtagta

SEQ ID NO: 301 - DNA, 24 bp, Synthetic construct
  source 1..24: mol_type = other DNA
ccaatacttctttatcttcaactc

SEQ ID NO: 302 - DNA, 24 bp, Synthetic construct
  source 1..24: mol_type = other DNA
agttgaagataaagaagtattgga

SEQ ID NO: 303 - DNA, 24 bp, Synthetic construct
  source 1..24: mol_type = other DNA
ctctcaacttttcctccatggaaa

SEQ ID NO: 304 - DNA, 24 bp, Synthetic construct
  source 1..24: mol_type = other DNA
ttccatggaggaaaagttgagagg

SEQ ID NO: 305 - DNA, 24 bp, Synthetic construct
  source 1..24: mol_type = other DNA
caaatgttcaaagtttcatgcaag

SEQ ID NO: 306 - DNA, 24 bp, Synthetic construct
  source 1..24: mol_type = other DNA
ttgcatgaaactttgaacatttgg

SEQ ID NO: 307 - DNA, 24 bp, Synthetic construct
  source 1..24: mol_type = other DNA
ataataacggtgaccaaaacgttg

SEQ ID NO: 308 - DNA, 24 bp, Synthetic construct
  source 1..24: mol_type = other DNA
aacgttttggtcaccgttattatt

SEQ ID NO: 309 - DNA, 24 bp, Synthetic construct
  source 1..24: mol_type = other DNA
ttctgttgatcgaagaaatgttct

SEQ ID NO: 310 - DNA, 24 bp, Synthetic construct
  source 1..24: mol_type = other DNA
gaacatttcttcgatcaacagaat

SEQ ID NO: 311 - DNA, 24 bp, Synthetic construct
  source 1..24: mol_type = other DNA
ttaggaggtctatatggtgttgct

SEQ ID NO: 312 - DNA, 24 bp, Synthetic construct
  source 1..24: mol_type = other DNA
gcaacaccatatagacctcctaaa

SEQ ID NO: 313 - DNA, 24 bp, Synthetic construct
  source 1..24: mol_type = other DNA
taccattagctgcatccgctgctc

SEQ ID NO: 314 - DNA, 24 bp, Synthetic construct
  source 1..24: mol_type = other DNA
agcagcggatgcagctaatggtat

SEQ ID NO: 315 - DNA, 24 bp, Synthetic construct
  source 1..24: mol_type = other DNA
gatcaaaggatggctaaatttttc

SEQ ID NO: 316 - DNA, 24 bp, Synthetic construct
  source 1..24: mol_type = other DNA
aaaaatttagccatcctttgatca

SEQ ID NO: 317 - DNA, 24 bp, Synthetic construct
  source 1..24: mol_type = other DNA
ttgaacttgaggatcaatatttcc

SEQ ID NO: 318 - DNA, 24 bp, Synthetic construct
  source 1..24: mol_type = other DNA
gaaatattgatcctcaagttcaaa

SEQ ID NO: 319 - DNA, 24 bp, Synthetic construct
  source 1..24: mol_type = other DNA
gagagtgagtaattactccaagat

SEQ ID NO: 320 - DNA, 24 bp, Synthetic construct
  source 1..24: mol_type = other DNA
tcttggagtaattactcactctct

SEQ ID NO: 321 - DNA, 24 bp, Synthetic construct
  source 1..24: mol_type = other DNA
acaattatcaccaacgtgttacac

SEQ ID NO: 322 - DNA, 24 bp, Synthetic construct
  source 1..24: mol_type = other DNA
tgtaacacgttggtgataattgta

SEQ ID NO: 323 - DNA, 24 bp, Synthetic construct
  source 1..24: mol_type = other DNA
gctacatataccttcaccattttg

SEQ ID NO: 324 - DNA, 24 bp, Synthetic construct
  source 1..24: mol_type = other DNA
aaaatggtgaaggtatatgtagca

SEQ ID NO: 325 - DNA, 24 bp, Synthetic construct
  source 1..24: mol_type = other DNA
gcaactcttctaacaaaaaatcac

SEQ ID NO: 326 - DNA, 24 bp, Synthetic construct
  source 1..24: mol_type = other DNA
tgattttttgttagaagagttgca

SEQ ID NO: 327 - DNA, 24 bp, Synthetic construct
  source 1..24: mol_type = other DNA
aacacaatgtcttcttctagtact

SEQ ID NO: 328 - DNA, 24 bp, Synthetic construct
  source 1..24: mol_type = other DNA
gtactagaagaagacattgtgttg

SEQ ID NO: 329 - DNA, 24 bp, Synthetic construct
  source 1..24: mol_type = other DNA
ttccattatgcaccaacaaatccc

SEQ ID NO: 330 - DNA, 24 bp, Synthetic construct
  source 1..24: mol_type = other DNA
ggatttgttggtgcataatggaag

SEQ ID NO: 331 - DNA, 24 bp, Synthetic construct
  source 1..24: mol_type = other DNA
ttccttcaccaccaacaactcatc

SEQ ID NO: 332 - DNA, 24 bp, Synthetic construct
  source 1..24: mol_type = other DNA
atgagttgttggtggtgaaggaag

SEQ ID NO: 333 - DNA, 24 bp, Synthetic construct
  source 1..24: mol_type = other DNA
tcaaaaccctctcaacttttcctc

SEQ ID NO: 334 - DNA, 24 bp, Synthetic construct
  source 1..24: mol_type = other DNA
aggaaaagttgagagggttttgat

SEQ ID NO: 335 - DNA, 24 bp, Synthetic construct
  source 1..24: mol_type = other DNA
gacgtaatcaaagtttcaaggttt

SEQ ID NO: 336 - DNA, 24 bp, Synthetic construct
  source 1..24: mol_type = other DNA
aaccttgaaactttgattacgtct

SEQ ID NO: 337 - DNA, 24 bp, Synthetic construct
  source 1..24: mol_type = other DNA
cgtcaacaataatgttggcgagca

SEQ ID NO: 338 - DNA, 24 bp, Synthetic construct
  source 1..24: mol_type = other DNA
gctcgccaacattattgttgacgt

SEQ ID NO: 339 - DNA, 24 bp, Synthetic construct
  source 1..24: mol_type = other DNA
aaccttgacgctgttgataggcga

SEQ ID NO: 340 - DNA, 24 bp, Synthetic construct
  source 1..24: mol_type = other DNA
cgcctatcaacagcgtcaaggttt

SEQ ID NO: 341 - DNA, 24 bp, Synthetic construct
  source 1..24: mol_type = other DNA
ttttagggttaggaggtctttatg

SEQ ID NO: 342 - DNA, 24 bp, Synthetic construct
  source 1..24: mol_type = other DNA
ataaagacctcctaaccctaaaag

SEQ ID NO: 343 - DNA, 24 bp, Synthetic construct
  source 1..24: mol_type = other DNA
taatcttgcaccattagcctctgc

SEQ ID NO: 344 - DNA, 24 bp, Synthetic construct
  source 1..24: mol_type = other DNA
cagaggctaatggtgcaagattag

SEQ ID NO: 345 - DNA, 24 bp, Synthetic construct
  source 1..24: mol_type = other DNA
tgcaaaagaaaataggatctgcat

SEQ ID NO: 346 - DNA, 24 bp, Synthetic construct
  source 1..24: mol_type = other DNA
tgcagatcctattttcttttgcat

SEQ ID NO: 347 - DNA, 24 bp, Synthetic construct
  source 1..24: mol_type = other DNA
atacaaaaccatttcaaaactgcg

SEQ ID NO: 348 - DNA, 24 bp, Synthetic construct
  source 1..24: mol_type = other DNA
gcagttttgaaatggttttgtatt

SEQ ID NO: 349 - DNA, 24 bp, Synthetic construct
  source 1..24: mol_type = other DNA
cgtttgtactaggtacatgaattt

SEQ ID NO: 350 - DNA, 24 bp, Synthetic construct
  source 1..24: mol_type = other DNA
aattcatgtacctagtacaaacgt

SEQ ID NO: 351 - DNA, 24 bp, Synthetic construct
  source 1..24: mol_type = other DNA
aaatactgatgaaacgctgcaaag

SEQ ID NO: 352 - DNA, 24 bp, Synthetic construct
  source 1..24: mol_type = other DNA
tttgcagcgtttcatcagtattta

SEQ ID NO: 353 - DNA, 24 bp, Synthetic construct
  source 1..24: mol_type = other DNA
actcatcccagcaatggcttcttc

SEQ ID NO: 354 - DNA, 24 bp, Synthetic construct
  source 1..24: mol_type = other DNA
aagaagccattgctgggatgagtg

SEQ ID NO: 355 - DNA, 24 bp, Synthetic construct
  source 1..24: mol_type = other DNA
cctttgtgcactaccaatattccc

SEQ ID NO: 356 - DNA, 24 bp, Synthetic construct
  source 1..24: mol_type = other DNA
ggaatattggtagtgcacaaaggt

SEQ ID NO: 357 - DNA, 24 bp, Synthetic construct
  source 1..24: mol_type = other DNA
tctccaataataccaactcatctt

SEQ ID NO: 358 - DNA, 24 bp, Synthetic construct
  source 1..24: mol_type = other DNA
agatgagttggtattattggagaa

SEQ ID NO: 359 - DNA, 24 bp, Synthetic construct
  source 1..24: mol_type = other DNA
aaaaccctctcagcttttcctcca

SEQ ID NO: 360 - DNA, 24 bp, Synthetic construct
  source 1..24: mol_type = other DNA
ggaggaaaagctgagagggttttg

SEQ ID NO: 361 - DNA, 24 bp, Synthetic construct
  source 1..24: mol_type = other DNA
cgtagtcaaagtttcaaggtttca

SEQ ID NO: 362 - DNA, 24 bp, Synthetic construct
  source 1..24: mol_type = other DNA
gaaaccttgaaactttgactacgc

SEQ ID NO: 363 - DNA, 24 bp, Synthetic construct
  source 1..24: mol_type = other DNA
atagcgagcatgacaaaaataacc

SEQ ID NO: 364 - DNA, 24 bp, Synthetic construct
  source 1..24: mol_type = other DNA
gttatttttgtcatgctcgctata

SEQ ID NO: 365 - DNA, 24 bp, Synthetic construct
  source 1..24: mol_type = other DNA
cgatgctgttgataggagaaatgt

SEQ ID NO: 366 - DNA, 24 bp, Synthetic construct
  source 1..24: mol_type = other DNA
catttctcctatcaacagcatcgt

SEQ ID NO: 367 - DNA, 24 bp, Synthetic construct
  source 1..24: mol_type = other DNA
gggttaggaggtctttatggtgct

SEQ ID NO: 368 - DNA, 24 bp, Synthetic construct
  source 1..24: mol_type = other DNA
gcaccataaagacctcctaaccct

SEQ ID NO: 369 - DNA, 24 bp, Synthetic construct
  source 1..24: mol_type = other DNA
ttgcaccattagccactgctgctc

SEQ ID NO: 370 - DNA, 24 bp, Synthetic construct
  source 1..24: mol_type = other DNA
agcagcagtggctaatggtgcaag

SEQ ID NO: 371 - DNA, 24 bp, Synthetic construct
  source 1..24: mol_type = other DNA
accacctgatttgaaaacttgtag

SEQ ID NO: 372 - DNA, 24 bp, Synthetic construct
  source 1..24: mol_type = other DNA
tacaagttttcaaatcaggtggtg

SEQ ID NO: 373 - DNA, 24 bp, Synthetic construct
  source 1..24: mol_type = other DNA
actgtaactcctggtggtccagca

SEQ ID NO: 374 - DNA, 24 bp, Synthetic construct
  source 1..24: mol_type = other DNA
gctggaccaccaggagttacagtg

SEQ ID NO: 375 - DNA, 24 bp, Synthetic construct
  source 1..24: mol_type = other DNA
tatgtgctcacgtggacacattac

SEQ ID NO: 376 - DNA, 24 bp, Synthetic construct
  source 1..24: mol_type = other DNA
taatgtgtccacgtgagcacatag

SEQ ID NO: 377 - DNA, 24 bp, Synthetic construct
  source 1..24: mol_type = other DNA
gatgcaatatttatgatgttcacg

SEQ ID NO: 378 - DNA, 24 bp, Synthetic construct
  source 1..24: mol_type = other DNA
gtgaacatcataaatattgcatca

SEQ ID NO: 379 - DNA, 24 bp, Synthetic construct
  source 1..24: mol_type = other DNA
attatattctcgacaagttgaacg

SEQ ID NO: 380 - DNA, 24 bp, Synthetic construct
  source 1..24: mol_type = other DNA
gttcaacttgtcgagaatataatt

SEQ ID NO: 381 - DNA, 24 bp, Synthetic construct
  source 1..24: mol_type = other DNA
ggaaatgatggagattattatggt

SEQ ID NO: 382 - DNA, 24 bp, Synthetic construct
  source 1..24: mol_type = other DNA
ccataataatctccatcatttcca

SEQ ID NO: 383 - DNA, 24 bp, Synthetic construct
  source 1..24: mol_type = other DNA
tcttcctcacaaggtaattacaaa

SEQ ID NO: 384 - DNA, 24 bp, Synthetic construct
  source 1..24: mol_type = other DNA
ttgtaattaccttgtgaggaagaa

SEQ ID NO: 385 - DNA, 24 bp, Synthetic construct
  source 1..24: mol_type = other DNA
ccttagcttgctccatattattct

SEQ ID NO: 386 - DNA, 24 bp, Synthetic construct
  source 1..24: mol_type = other DNA
gaataatatggagcaagctaagga

SEQ ID NO: 387 - DNA, 24 bp, Synthetic construct
  source 1..24: mol_type = other DNA
tgctagccctagatgttcatgaat

SEQ ID NO: 388 - DNA, 24 bp, Synthetic construct
  source 1..24: mol_type = other DNA
ttcatgaacatctagggctagcat

SEQ ID NO: 389 - DNA, 24 bp, Synthetic construct
  source 1..24: mol_type = other DNA
caagcaaaaaaatgtcttccattc

SEQ ID NO: 390 - DNA, 24 bp, Synthetic construct
  source 1..24: mol_type = other DNA
aatggaagacatttttttgcttgt

SEQ ID NO: 391 - DNA, 24 bp, Synthetic construct
  source 1..24: mol_type = other DNA
cactaccaatactctctcttcttc

SEQ ID NO: 392 - DNA, 24 bp, Synthetic construct
  source 1..24: mol_type = other DNA
aagaagagagagtattggtagtgg

SEQ ID NO: 393 - DNA, 24 bp, Synthetic construct
  source 1..24: mol_type = other DNA
accactttttccaacttgcattct

SEQ ID NO: 394 - DNA, 24 bp, Synthetic construct
  source 1..24: mol_type = other DNA
gaatgcaagttggaaaaagtggtg

SEQ ID NO: 395 - DNA, 24 bp, Synthetic construct
  source 1..24: mol_type = other DNA
gcaaaaacatcaaaaatttcctcc

SEQ ID NO: 396 - DNA, 24 bp, Synthetic construct
  source 1..24: mol_type = other DNA
gaggaaatttttgatgtttttgca

SEQ ID NO: 397 - DNA, 24 bp, Synthetic construct
  source 1..24: mol_type = other DNA
agcataatgtccatcgtaatttcc

SEQ ID NO: 398 - DNA, 24 bp, Synthetic construct
  source 1..24: mol_type = other DNA
gaaattacgatggacattatgctt

SEQ ID NO: 399 - DNA, 24 bp, Synthetic construct
  source 1..24: mol_type = other DNA
gcaaaaccatagatgataatagtc

SEQ ID NO: 400 - DNA, 24 bp, Synthetic construct
  source 1..24: mol_type = other DNA
actattatcatctatggttttgca

SEQ ID NO: 401 - DNA, 24 bp, Synthetic construct
  source 1..24: mol_type = other DNA
caataactcacccatcgacatttc

SEQ ID NO: 402 - DNA, 24 bp, Synthetic construct
  source 1..24: mol_type = other DNA
aaatgtcgatgggtgagttattgt

SEQ ID NO: 403 - DNA, 24 bp, Synthetic construct
  source 1..24: mol_type = other DNA
acaatatgatcgatagaagaaacg

SEQ ID NO: 404 - DNA, 24 bp, Synthetic construct
  source 1..24: mol_type = other DNA
gtttcttctatcgatcatattgtt

SEQ ID NO: 405 - DNA, 24 bp, Synthetic construct
  source 1..24: mol_type = other DNA
tcggcctgccgagcctagacaata

SEQ ID NO: 406 - DNA, 24 bp, Synthetic construct
  source 1..24: mol_type = other DNA
attgtctaggctcggcaggccgac

SEQ ID NO: 407 - DNA, 24 bp, Synthetic construct
  source 1..24: mol_type = other DNA
tctcctctcgatggggtctctccc

SEQ ID NO: 408 - DNA, 24 bp, Synthetic construct
  source 1..24: mol_type = other DNA
ggagagaccccatcgagaggagag

SEQ ID NO: 409 - DNA, 24 bp, Synthetic construct
  source 1..24: mol_type = other DNA
atccaggcgccgccggtgaccttc

SEQ ID NO: 410 - DNA, 24 bp, Synthetic construct
  source 1..24: mol_type = other DNA
aaggtcaccggcggcgcctggatc

SEQ ID NO: 411 - DNA, 24 bp, Synthetic construct
  source 1..24: mol_type = other DNA
gcgcacgcgcggatcattcgtccc

SEQ ID NO: 412 - DNA, 24 bp, Synthetic construct
  source 1..24: mol_type = other DNA
ggacgaatgatccgcgcgtgcgca

SEQ ID NO: 413 - DNA, 24 bp, Synthetic construct
  source 1..24: mol_type = other DNA
acagtctgacacgttagatagaga

SEQ ID NO: 414 - DNA, 24 bp, Synthetic construct
  source 1..24: mol_type = other DNA
ctctatctaacgtgtcagactgtc

SEQ ID NO: 415 - DNA, 24 bp, Synthetic construct
  source 1..24: mol_type = other DNA
atgcgccaggtcgtggaccgtccc

SEQ ID NO: 416 - DNA, 24 bp, Synthetic construct
  source 1..24: mol_type = other DNA
ggacggtccacgacctggcgcatg

SEQ ID NO: 417 - DNA, 24 bp, Synthetic construct
  source 1..24: mol_type = other DNA
agagaacactgccgtcggttttta

SEQ ID NO: 418 - DNA, 24 bp, Synthetic construct
  source 1..24: mol_type = other DNA
aaaaaccgacggcagtgttctctg

SEQ ID NO: 419 - DNA, 24 bp, Synthetic construct
  source 1..24: mol_type = other DNA
ttgacgttcgaaaagatgtcaaca

SEQ ID NO: 420 - DNA, 24 bp, Synthetic construct
  source 1..24: mol_type = other DNA
gttgacatcttttcgaacgtcaat

SEQ ID NO: 421 - DNA, 24 bp, Synthetic construct
  source 1..24: mol_type = other DNA
agtttttttatacaactgagagag

SEQ ID NO: 422 - DNA, 24 bp, Synthetic construct
  source 1..24: mol_type = other DNA
tctctcagttgtataaaaaaactc

SEQ ID NO: 423 - DNA, 24 bp, Synthetic construct
  source 1..24: mol_type = other DNA
gagtgagttaaatggcaacaaaca

SEQ ID NO: 424 - DNA, 24 bp, Synthetic construct
  source 1..24: mol_type = other DNA
gtttgttgccatttaactcactcg

SEQ ID NO: 425 - DNA, 24 bp, Synthetic construct
  source 1..24: mol_type = other DNA
ggaaaaaaactatgagatgtcatc

SEQ ID NO: 426 - DNA, 24 bp, Synthetic construct
  source 1..24: mol_type = other DNA
atgacatctcatagtttttttcct

SEQ ID NO: 427 - DNA, 24 bp, Synthetic construct
  source 1..24: mol_type = other DNA
atgacggtaaataaatatggatgg

SEQ ID NO: 428 - DNA, 24 bp, Synthetic construct
  source 1..24: mol_type = other DNA
catccatatttatttaccgtcata

SEQ ID NO: 429 - DNA, 24 bp, Synthetic construct
  source 1..24: mol_type = other DNA
ctaaaacgaaaagtggcaaaacct

SEQ ID NO: 430 - DNA, 24 bp, Synthetic construct
  source 1..24: mol_type = other DNA
ggttttgccacttttcgttttagt

SEQ ID NO: 431 - DNA, 24 bp, Synthetic construct
  source 1..24: mol_type = other DNA
gacgggtgtcgccgagtgcagccg

SEQ ID NO: 432 - DNA, 24 bp, Synthetic construct
  source 1..24: mol_type = other DNA
ggctgcactcggcgacacccgtcc

SEQ ID NO: 433 - DNA, 24 bp, Synthetic construct
  source 1..24: mol_type = other DNA
acccccaccgatgtcctgagattg

SEQ ID NO: 434 - DNA, 24 bp, Synthetic construct
  source 1..24: mol_type = other DNA
aatctcaggacatcggtgggggtt

SEQ ID NO: 435 - DNA, 24 bp, Synthetic construct
  source 1..24: mol_type = other DNA
aagccgcaggcagcatctgcatct

SEQ ID NO: 436 - DNA, 24 bp, Synthetic construct
  source 1..24: mol_type = other DNA
gatgcagatgctgcctgcggcttc

SEQ ID NO: 437 - DNA, 24 bp, Synthetic construct
  source 1..24: mol_type = other DNA
ctccgctccgcctactgctgctgg

SEQ ID NO: 438 - DNA, 24 bp, Synthetic construct
  source 1..24: mol_type = other DNA
cagcagcagtaggcggagcggagg

SEQ ID NO: 439 - DNA, 24 bp, Synthetic construct
  source 1..24: mol_type = other DNA
ggcggagaaggaggcccttgcgcc

SEQ ID NO: 440 - DNA, 24 bp, Synthetic construct
  source 1..24: mol_type = other DNA
gcgcaagggcctccttctccgcct

SEQ ID NO: 441 - DNA, 24 bp, Synthetic construct
  source 1..24: mol_type = other DNA
gccggatcagagccggtaagacca

SEQ ID NO: 442 - DNA, 24 bp, Synthetic construct
  source 1..24: mol_type = other DNA
ggtcttaccggctctgatccggcc

SEQ ID NO: 443 - DNA, 24 bp, Synthetic construct
  source 1..24: mol_type = other DNA
cgctcctcctcgctggttgctttc

SEQ ID NO: 444 - DNA, 24 bp, Synthetic construct
  source 1..24: mol_type = other DNA
aaagcaaccagcgaggaggagcgt

SEQ ID NO: 445 - DNA, 24 bp, Synthetic construct
  source 1..24: mol_type = other DNA
cgccggtattcccagtccgtgtgc

SEQ ID NO: 446 - DNA, 24 bp, Synthetic construct
  source 1..24: mol_type = other DNA
cacacggactgggaataccggcgg

SEQ ID NO: 447 - DNA, 24 bp, Synthetic construct
  source 1..24: mol_type = other DNA
gtctgctcccgtcgctgcctagat

SEQ ID NO: 448 - DNA, 24 bp, Synthetic construct
  source 1..24: mol_type = other DNA
tctaggcagcgacgggagcagaca

SEQ ID NO: 449 - DNA, 24 bp, Synthetic construct
  source 1..24: mol_type = other DNA
ggatctttcgtgcatggcggcaga

SEQ ID NO: 450 - DNA, 24 bp, Synthetic construct
  source 1..24: mol_type = other DNA
ctgccgccatgcacgaaagatcct

SEQ ID NO: 451 - DNA, 24 bp, Synthetic construct
  source 1..24: mol_type = other DNA
cccccccccccccccccccccccc

SEQ ID NO: 452 - DNA, 24 bp, Synthetic construct
  source 1..24: mol_type = other DNA
gggggggggggggggggggggggg

SEQ ID NO: 453 - DNA, 24 bp, Synthetic construct
  source 1..24: mol_type = other DNA
cgtgtatacgagttttctctaggc

SEQ ID NO: 454 - DNA, 24 bp, Synthetic construct
  source 1..24: mol_type = other DNA
cctagagaaaactcgtatacacgg

SEQ ID NO: 455 - DNA, 24 bp, Synthetic construct
  source 1..24: mol_type = other DNA
catgttggattggattgcgctagt

SEQ ID NO: 456 - DNA, 24 bp, Synthetic construct
  source 1..24: mol_type = other DNA
ctagcgcaatccaatccaacatgc

SEQ ID NO: 457 - DNA, 24 bp, Synthetic construct
  source 1..24: mol_type = other DNA
gaggtgccggccgtacccatcctc

SEQ ID NO: 458 - DNA, 24 bp, Synthetic construct
  source 1..24: mol_type = other DNA
aggatgggtacggccggcacctct

SEQ ID NO: 459 - DNA, 24 bp, Synthetic construct
  source 1..24: mol_type = other DNA
agaaaaaaggcccagtcatttttg

SEQ ID NO: 460 - DNA, 24 bp, Synthetic construct
  source 1..24: mol_type = other DNA
aaaaatgactgggccttttttctg

SEQ ID NO: 461 - DNA, 24 bp, Synthetic construct
  source 1..24: mol_type = other DNA
atttatttttacagctgccacatg

SEQ ID NO: 462 - DNA, 24 bp, Synthetic construct
  source 1..24: mol_type = other DNA
atgtggcagctgtaaaaataaata

SEQ ID NO: 463 - DNA, 24 bp, Synthetic construct
  source 1..24: mol_type = other DNA
tttttgttggggttttactactac

SEQ ID NO: 464 - DNA, 24 bp, Synthetic construct
  source 1..24: mol_type = other DNA
tagtagtaaaaccccaacaaaaac

SEQ ID NO: 465 - DNA, 24 bp, Synthetic construct
  source 1..24: mol_type = other DNA
aactgttttgtcaaatactgtaac

SEQ ID NO: 466 - DNA, 24 bp, Synthetic construct
  source 1..24: mol_type = other DNA
ttacagtatttgacaaaacagttc

SEQ ID NO: 467 - DNA, 24 bp, Synthetic construct
  source 1..24: mol_type = other DNA
aaagctgtttgtaggagtgaagct

SEQ ID NO: 468 - DNA, 24 bp, Synthetic construct
  source 1..24: mol_type = other DNA
gcttcactcctacaaacagcttta

SEQ ID NO: 469 - DNA, 24 bp, Synthetic construct
  source 1..24: mol_type = other DNA
aaacagaacttcatattgttccag

SEQ ID NO: 470 - DNA, 24 bp, Synthetic construct
  source 1..24: mol_type = other DNA
tggaacaatatgaagttctgttta

SEQ ID NO: 471 - DNA, 24 bp, Synthetic construct
  source 1..24: mol_type = other DNA
ttccaacaaaaaaattgcaattcg

SEQ ID NO: 472 - DNA, 24 bp, Synthetic construct
  source 1..24: mol_type = other DNA
gaattgcaatttttttgttggaac

SEQ ID NO: 473 - DNA, 24 bp, Synthetic construct
  source 1..24: mol_type = other DNA
gctaccagtacagcgctagatgga

SEQ ID NO: 474 - DNA, 24 bp, Synthetic construct
  source 1..24: mol_type = other DNA
ccatctagcgctgtactggtagcc

SEQ ID NO: 475 - DNA, 24 bp, Synthetic construct
  source 1..24: mol_type = other DNA
cgaacatgaaacgtttactttttc

SEQ ID NO: 476 - DNA, 24 bp, Synthetic construct
  source 1..24: mol_type = other DNA
aaaaagtaaacgtttcatgttcgc

SEQ ID NO: 477 - DNA, 24 bp, Synthetic construct
  source 1..24: mol_type = other DNA
tgtttgatggatcacatttatctc

SEQ ID NO: 478 - DNA, 24 bp, Synthetic construct
  source 1..24: mol_type = other DNA
agataaatgtgatccatcaaacat

SEQ ID NO: 479 - DNA, 24 bp, Synthetic construct
  source 1..24: mol_type = other DNA
tgttggataccggtactttttacg

SEQ ID NO: 480 - DNA, 24 bp, Synthetic construct
  source 1..24: mol_type = other DNA
gtaaaaagtaccggtatccaacac

SEQ ID NO: 481 - DNA, 24 bp, Synthetic construct
  source 1..24: mol_type = other DNA
gtacaggggccactggctatatat

SEQ ID NO: 482 - DNA, 24 bp, Synthetic construct
  source 1..24: mol_type = other DNA
tatatagccagtggcccctgtaca

SEQ ID NO: 483 - DNA, 24 bp, Synthetic construct
  source 1..24: mol_type = other DNA
cactccattaatttccagggatgc

SEQ ID NO: 484 - DNA, 24 bp, Synthetic construct
  source 1..24: mol_type = other DNA
catccctggaaattaatggagtga

SEQ ID NO: 485 - DNA, 24 bp, Synthetic construct
  source 1..24: mol_type = other DNA
ctctctctgctacatacatccatt

SEQ ID NO: 486 - DNA, 24 bp, Synthetic construct
  source 1..24: mol_type = other DNA
atggatgtatgtagcagagagaga

SEQ ID NO: 487 - DNA, 24 bp, Synthetic construct
  source 1..24: mol_type = other DNA
ttttttgtggaattttgcacttgg

SEQ ID NO: 488 - DNA, 24 bp, Synthetic construct
  source 1..24: mol_type = other DNA
caagtgcaaaattccacaaaaaaa

SEQ ID NO: 489 - DNA, 24 bp, Synthetic construct
  source 1..24: mol_type = other DNA
tctcagtttaatttggaggatcaa

SEQ ID NO: 490 - DNA, 24 bp, Synthetic construct
  source 1..24: mol_type = other DNA
tgatcctccaaattaaactgagaa

SEQ ID NO: 491 - DNA, 24 bp, Synthetic construct
  source 1..24: mol_type = other DNA
agacatacatggcggatcctctgg

SEQ ID NO: 492 - DNA, 24 bp, Synthetic construct
  source 1..24: mol_type = other DNA
cagaggatccgccatgtatgtctc

SEQ ID NO: 493 - DNA, 24 bp, Synthetic construct
  source 1..24: mol_type = other DNA
aaggtcttagcggactacttggtg

SEQ ID NO: 494 - DNA, 24 bp, Synthetic construct
  source 1..24: mol_type = other DNA
accaagtagtccgctaagaccttc

SEQ ID NO: 495 - DNA, 24 bp, Synthetic construct
  source 1..24: mol_type = other DNA
cttcgtggcccttccaatatctgc

SEQ ID NO: 496 - DNA, 24 bp, Synthetic construct
  source 1..24: mol_type = other DNA
cagatattggaagggccacgaaga

SEQ ID NO: 497 - DNA, 24 bp, Synthetic construct
  source 1..24: mol_type = other DNA
tggtgaacacgtggtccgccatga

SEQ ID NO: 498 - DNA, 24 bp, Synthetic construct
  source 1..24: mol_type = other DNA
catggcggaccacgtgttcaccag

SEQ ID NO: 499 - DNA, 24 bp, Synthetic construct
  source 1..24: mol_type = other DNA
gaacaccaggagaacgtagagggt

SEQ ID NO: 500 - DNA, 24 bp, Synthetic construct
  source 1..24: mol_type = other DNA
ccctctacgttctcctggtgttcc

SEQ ID NO: 501 - DNA, 24 bp, Synthetic construct
  source 1..24: mol_type = other DNA
atccgaagagggacggcctcgtct

SEQ ID NO: 502 - DNA, 24 bp, Synthetic construct
  source 1..24: mol_type = other DNA
gacgaggccgtccctcttcggatc

SEQ ID NO: 503 - DNA, 24 bp, Synthetic construct
  source 1..24: mol_type = other DNA
gtcgtgggcatgcgcaacgtggac

SEQ ID NO: 504 - DNA, 24 bp, Synthetic construct
  source 1..24: mol_type = other DNA
tccacgttgcgcatgcccacgacc

SEQ ID NO: 505 - DNA, 24 bp, Synthetic construct
  source 1..24: mol_type = other DNA
ggtcgacctctccgcgttgaggaa

SEQ ID NO: 506 - DNA, 24 bp, Synthetic construct
  source 1..24: mol_type = other DNA
tcctcaacgcggagaggtcgacct

SEQ ID NO: 507 - DNA, 24 bp, Synthetic construct
  source 1..24: mol_type = other DNA
ggatggtcgccagggtggagcctc

SEQ ID NO: 508 - DNA, 24 bp, Synthetic construct
  source 1..24: mol_type = other DNA
aggctccaccctggcgaccatcct

SEQ ID NO: 509 - DNA, 24 bp, Synthetic construct
  source 1..24: mol_type = other DNA
aaggctacctgccccatgaacctc

SEQ ID NO: 510 - DNA, 24 bp, Synthetic construct
  source 1..24: mol_type = other DNA
aggttcatggggcaggtagccttg

SEQ ID NO: 511 - DNA, 24 bp, Synthetic construct
  source 1..24: mol_type = other DNA
ggacgaccttaccgtcggtggtct

SEQ ID NO: 512 - DNA, 24 bp, Synthetic construct
  source 1..24: mol_type = other DNA
gaccaccgacggtaaggtcgtcca

SEQ ID NO: 513 - DNA, 24 bp, Synthetic construct
  source 1..24: mol_type = other DNA
ggagctctcacgtctacggcctct

SEQ ID NO: 514 - DNA, 24 bp, Synthetic construct
  source 1..24: mol_type = other DNA
gaggccgtagacgtgagagctccc

SEQ ID NO: 515 - DNA, 24 bp, Synthetic construct
  source 1..24: mol_type = other DNA
gaggtcgttcttgcggatgggcag

SEQ ID NO: 516 - DNA, 24 bp, Synthetic construct
  source 1..24: mol_type = other DNA
tgcccatccgcaagaacgacctcc

SEQ ID NO: 517 - DNA, 24 bp, Synthetic construct
  source 1..24: mol_type = other DNA
cgagcactccgacctcttctatgg

SEQ ID NO: 518 - DNA, 24 bp, Synthetic construct
  source 1..24: mol_type = other DNA
catagaagaggtcggagtgctcgt

SEQ ID NO: 519 - DNA, 24 bp, Synthetic construct
  source 1..24: mol_type = other DNA
tcgggctcctggtttcggctgaga

SEQ ID NO: 520 - DNA, 24 bp, Synthetic construct
  source 1..24: mol_type = other DNA
ctcagccgaaaccaggagcccgat

SEQ ID NO: 521 - DNA, 24 bp, Synthetic construct
  source 1..24: mol_type = other DNA
tacatgaggctcacgtacactcct

SEQ ID NO: 522 - DNA, 24 bp, Synthetic construct
  source 1..24: mol_type = other DNA
ggagtgtacgtgagcctcatgtac

SEQ ID NO: 523 - DNA, 24 bp, Synthetic construct
  source 1..24: mol_type = other DNA
cgcggaggcttatgctgattcgtt

SEQ ID NO: 524 - DNA, 24 bp, Synthetic construct
  source 1..24: mol_type = other DNA
acgaatcagcataagcctccgcga

SEQ ID NO: 525 - DNA, 24 bp, Synthetic construct
  source 1..24: mol_type = other DNA
cacatgaacccgtatcgcgagatg

SEQ ID NO: 526 - DNA, 24 bp, Synthetic construct
  source 1..24: mol_type = other DNA
atctcgcgatacgggttcatgtgt

SEQ ID NO: 527 - DNA, 24 bp, Synthetic construct
  source 1..24: mol_type = other DNA
tctttatcacagtggatgcatatg

SEQ ID NO: 528 - DNA, 24 bp, Synthetic construct
  source 1..24: mol_type = other DNA
atatgcatccactgtgataaagaa

SEQ ID NO: 529 - DNA, 24 bp, Synthetic construct
  source 1..24: mol_type = other DNA
acagatggttagcgagtgacagta

SEQ ID NO: 530 - DNA, 24 bp, Synthetic construct
  source 1..24: mol_type = other DNA
actgtcactcgctaaccatctgtt

SEQ ID NO: 531 - DNA, 24 bp, Synthetic construct
  source 1..24: mol_type = other DNA
agttgtccgacacttcatcggtaa

SEQ ID NO: 532 - DNA, 24 bp, Synthetic construct
  source 1..24: mol_type = other DNA
taccgatgaagtgtcggacaactt

SEQ ID NO: 533 - DNA, 24 bp, Synthetic construct
  source 1..24: mol_type = other DNA
accgagtgaatggaagaaaaacga

SEQ ID NO: 534 - DNA, 24 bp, Synthetic construct
  source 1..24: mol_type = other DNA
cgtttttcttccattcactcggtt

SEQ ID NO: 535 - DNA, 24 bp, Synthetic construct
  source 1..24: mol_type = other DNA
acagcaggttttcttaaaaaacgt

SEQ ID NO: 536 - DNA, 24 bp, Synthetic construct
  source 1..24: mol_type = other DNA
cgttttttaagaaaacctgctgtg

SEQ ID NO: 537 - DNA, 24 bp, Synthetic construct
  source 1..24: mol_type = other DNA
ttaagaagagaccaaaatatggtc

SEQ ID NO: 538 - DNA, 24 bp, Synthetic construct
  source 1..24: mol_type = other DNA
accatattttggtctcttcttaat

SEQ ID NO: 539 - DNA, 24 bp, Synthetic construct
  source 1..24: mol_type = other DNA
ttctaaacattagttctcatcacc

SEQ ID NO: 540 - DNA, 24 bp, Synthetic construct
  source 1..24: mol_type = other DNA
gtgatgagaactaatgtttagaaa

SEQ ID NO: 541 - DNA, 24 bp, Synthetic construct
  source 1..24: mol_type = other DNA
catctagtttgcaacggtccagtt

SEQ ID NO: 542 - DNA, 24 bp, Synthetic construct
  source 1..24: mol_type = other DNA
actggaccgttgcaaactagatgg

SEQ ID NO: 543 - DNA, 24 bp, Synthetic construct
  source 1..24: mol_type = other DNA
gactcgcagcgagagaattttttt

SEQ ID NO: 544 - DNA, 24 bp, Synthetic construct
  source 1..24: mol_type = other DNA
aaaaaattctctcgctgcgagtcc

SEQ ID NO: 545 - DNA, 24 bp, Synthetic construct
  source 1..24: mol_type = other DNA
attcactttcggacaaatcgaact

SEQ ID NO: 546 - DNA, 24 bp, Synthetic construct
  source 1..24: mol_type = other DNA
gttcgatttgtccgaaagtgaatt

SEQ ID NO: 547 - DNA, 24 bp, Synthetic construct
  source 1..24: mol_type = other DNA
aaccatgagaccttttcgccgcag

SEQ ID NO: 548 - DNA, 24 bp, Synthetic construct
  source 1..24: mol_type = other DNA
tgcggcgaaaaggtctcatggtta

SEQ ID NO: 549 - DNA, 24 bp, Synthetic construct
  source 1..24: mol_type = other DNA
ggccgttagattttagtgacgatg

SEQ ID NO: 550 - DNA, 24 bp, Synthetic construct
  source 1..24: mol_type = other DNA
atcgtcactaaaatctaacggccg

SEQ ID NO: 551 - DNA, 24 bp, Synthetic construct
  source 1..24: mol_type = other DNA
gcaacgtgccgcatgcatccattc

SEQ ID NO: 552 - DNA, 24 bp, Synthetic construct
  source 1..24: mol_type = other DNA
aatggatgcatgcggcacgttgcg

SEQ ID NO: 553 - DNA, 24 bp, Synthetic construct
  source 1..24: mol_type = other DNA
acagtacatgtaggagtactgtgc

SEQ ID NO: 554 - DNA, 24 bp, Synthetic construct
  source 1..24: mol_type = other DNA
cacagtactcctacatgtactgtg

SEQ ID NO: 555 - DNA, 24 bp, Synthetic construct
  source 1..24: mol_type = other DNA
acattcagtctctctcactagttg

SEQ ID NO: 556 - DNA, 24 bp, Synthetic construct
  source 1..24: mol_type = other DNA
aactagtgagagagactgaatgta

SEQ ID NO: 557 - DNA, 24 bp, Synthetic construct
  source 1..24: mol_type = other DNA
ctacaaagacatgagctgccggga

SEQ ID NO: 558 - DNA, 24 bp, Synthetic construct
  source 1..24: mol_type = other DNA
cccggcagctcatgtctttgtagc

SEQ ID NO: 559 - DNA, 24 bp, Synthetic construct
  source 1..24: mol_type = other DNA
gagcgagcgagcctgacggtctca

SEQ ID NO: 560 - DNA, 24 bp, Synthetic construct
  source 1..24: mol_type = other DNA
gagaccgtcaggctcgctcgctcc

SEQ ID NO: 561 - DNA, 24 bp, Synthetic construct
  source 1..24: mol_type = other DNA
actcccaagccaattattataaga

SEQ ID NO: 562 - DNA, 24 bp, Synthetic construct
  source 1..24: mol_type = other DNA
cttataataattggcttgggagtg

SEQ ID NO: 563 - DNA, 24 bp, Synthetic construct
  source 1..24: mol_type = other DNA
actccagctcttaaccaatccact

SEQ ID NO: 564 - DNA, 24 bp, Synthetic construct
  source 1..24: mol_type = other DNA
gtggattggttaagagctggagtt

SEQ ID NO: 565 - DNA, 24 bp, Synthetic construct
  source 1..24: mol_type = other DNA
cacctcctctgctttgctctgcca

SEQ ID NO: 566 - DNA, 24 bp, Synthetic construct
  source 1..24: mol_type = other DNA
ggcagagcaaagcagaggaggtgg

SEQ ID NO: 567 - DNA, 24 bp, Synthetic construct
  source 1..24: mol_type = other DNA
gggggcagaggagctccccctccc

SEQ ID NO: 568 - DNA, 24 bp, Synthetic construct
  source 1..24: mol_type = other DNA
ggagggggagctcctctgcccccc

SEQ ID NO: 569 - DNA, 24 bp, Synthetic construct
  source 1..24: mol_type = other DNA
tcgccatgtctagcagcgacccgg

SEQ ID NO: 570 - DNA, 24 bp, Synthetic construct
  source 1..24: mol_type = other DNA
cgggtcgctgctagacatggcgag

SEQ ID NO: 571 - DNA, 24 bp, Synthetic construct
  source 1..24: mol_type = other DNA
gcgcgtcgtcgttctcggttcgcc

SEQ ID NO: 572 - DNA, 24 bp, Synthetic construct
  source 1..24: mol_type = other DNA
gcgaaccgagaacgacgacgcgcg

SEQ ID NO: 573 - DNA, 24 bp, Synthetic construct
  source 1..24: mol_type = other DNA
ggcgacgagtgggcccggcccgag

SEQ ID NO: 574 - DNA, 24 bp, Synthetic construct
  source 1..24: mol_type = other DNA
tcgggccgggcccactcgtcgccg

SEQ ID NO: 575 - DNA, 24 bp, Synthetic construct
  source 1..24: mol_type = other DNA
atctgccgtctcccgcccaccagc

SEQ ID NO: 576 - DNA, 24 bp, Synthetic construct
  source 1..24: mol_type = other DNA
ctggtgggcgggagacggcagatg

SEQ ID NO: 577 - DNA, 24 bp, Synthetic construct
  source 1..24: mol_type = other DNA
agccgggcaaccggaagcagcaga

SEQ ID NO: 578 - DNA, 24 bp, Synthetic construct
  source 1..24: mol_type = other DNA
ctgctgcttccggttgcccggcta

SEQ ID NO: 579 - DNA, 24 bp, Synthetic construct
  source 1..24: mol_type = other DNA
cctgctcctgctggccgcagcagc

SEQ ID NO: 580 - DNA, 24 bp, Synthetic construct
  source 1..24: mol_type = other DNA
ctgctgcggccagcaggagcaggg

SEQ ID NO: 581 - DNA, 24 bp, Synthetic construct
  source 1..24: mol_type = other DNA
cgcctactacatctgccggtggcg

SEQ ID NO: 582 - DNA, 24 bp, Synthetic construct
  source 1..24: mol_type = other DNA
gccaccggcagatgtagtaggcgt

SEQ ID NO: 583 - DNA, 24 bp, Synthetic construct
  source 1..24: mol_type = other DNA
tcctccttcttcgcctccccctcc

SEQ ID NO: 584 - DNA, 24 bp, Synthetic construct
  source 1..24: mol_type = other DNA
gagggggaggcgaagaaggaggag

SEQ ID NO: 585 - DNA, 24 bp, Synthetic construct
  source 1..24: mol_type = other DNA
ggtttaaaaaagatttcttttttt

SEQ ID NO: 586 - DNA, 24 bp, Synthetic construct
  source 1..24: mol_type = other DNA
aaaaaagaaatcttttttaaacct

SEQ ID NO: 587 - DNA, 24 bp, Synthetic construct
  source 1..24: mol_type = other DNA
gtaatcgacacactaatgcaaaga

SEQ ID NO: 588 - DNA, 24 bp, Synthetic construct
  source 1..24: mol_type = other DNA
ctttgcattagtgtgtcgattact

SEQ ID NO: 589 - DNA, 24 bp, Synthetic construct
  source 1..24: mol_type = other DNA
aacatcttggacctaaataattgt

SEQ ID NO: 590 - DNA, 24 bp, Synthetic construct
  source 1..24: mol_type = other DNA
caattatttaggtccaagatgttt

SEQ ID NO: 591 - DNA, 24 bp, Synthetic construct
  source 1..24: mol_type = other DNA
ctttccattttcatctttaaatat

SEQ ID NO: 592 - DNA, 24 bp, Synthetic construct
  source 1..24: mol_type = other DNA
tatttaaagatgaaaatggaaagg

SEQ ID NO: 593 - DNA, 24 bp, Synthetic construct
  source 1..24: mol_type = other DNA
acaatttttttttgggctaaaatg

SEQ ID NO: 594 - DNA, 24 bp, Synthetic construct
  source 1..24: mol_type = other DNA
attttagcccaaaaaaaaattgtg

SEQ ID NO: 595 - DNA, 24 bp, Synthetic construct
  source 1..24: mol_type = other DNA
tggtggagttatgaccacatattg

SEQ ID NO: 596 - DNA, 24 bp, Synthetic construct
  source 1..24: mol_type = other DNA
aatatgtggtcataactccaccat

SEQ ID NO: 597 - DNA, 24 bp, Synthetic construct
  source 1..24: mol_type = other DNA
agtgctcaaaaggagagtctactg

SEQ ID NO: 598 - DNA, 24 bp, Synthetic construct
  source 1..24: mol_type = other DNA
agtagactctccttttgagcactt

SEQ ID NO: 599 - DNA, 24 bp, Synthetic construct
  source 1..24: mol_type = other DNA
ccaccacaagtactatgcaacaaa

SEQ ID NO: 600 - DNA, 24 bp, Synthetic construct
  source 1..24: mol_type = other DNA
ttgttgcatagtacttgtggtgga

SEQ ID NO: 601 - DNA, 24 bp, Synthetic construct
  source 1..24: mol_type = other DNA
aagaaaatggaaacttttctctct

SEQ ID NO: 602 - DNA, 24 bp, Synthetic construct
  source 1..24: mol_type = other DNA
gagagaaaagtttccattttcttg

SEQ ID NO: 603 - DNA, 24 bp, Synthetic construct
  source 1..24: mol_type = other DNA
cactagctgtttacatggtgagca

SEQ ID NO: 604 - DNA, 24 bp, Synthetic construct
  source 1..24: mol_type = other DNA
gctcaccatgtaaacagctagtga

SEQ ID NO: 605 - DNA, 24 bp, Synthetic construct
  source 1..24: mol_type = other DNA
agaaatacttagtatatatctata

SEQ ID NO: 606 - DNA, 24 bp, Synthetic construct
  source 1..24: mol_type = other DNA
atagatatatactaagtatttcta

SEQ ID NO: 607 - DNA, 24 bp, Synthetic construct
  source 1..24: mol_type = other DNA
acttttcattctgtaattctttaa

SEQ ID NO: 608 - DNA, 24 bp, Synthetic construct
  source 1..24: mol_type = other DNA
taaagaattacagaatgaaaagtg

SEQ ID NO: 609 - DNA, 24 bp, Synthetic construct
  source 1..24: mol_type = other DNA
ctgtttaaagcttgatttttttta

SEQ ID NO: 610 - DNA, 24 bp, Synthetic construct
  source 1..24: mol_type = other DNA
aaaaaaaatcaagctttaaacagt

SEQ ID NO: 611 - DNA, 24 bp, Synthetic construct
  source 1..24: mol_type = other DNA
atgttctgcttcatttgtgttgaa

SEQ ID NO: 612 - DNA, 24 bp, Synthetic construct
  source 1..24: mol_type = other DNA
tcaacacaaatgaagcagaacatg

SEQ ID NO: 613 - DNA, 24 bp, Synthetic construct
  source 1..24: mol_type = other DNA
attggggaactttcttgaatccag

SEQ ID NO: 614 - DNA, 24 bp, Synthetic construct
  source 1..24: mol_type = other DNA
tggattcaagaaagttccccaatt

SEQ ID NO: 615 - DNA, 24 bp, Synthetic construct
  source 1..24: mol_type = other DNA
catttgaagttttcttgaaacaaa

SEQ ID NO: 616 - DNA, 24 bp, Synthetic construct
  source 1..24: mol_type = other DNA
ttgtttcaagaaaacttcaaatgg

SEQ ID NO: 617 - DNA, 24 bp, Synthetic construct
  source 1..24: mol_type = other DNA
cattaccctgttggaaaaagatgg

SEQ ID NO: 618 - DNA, 24 bp, Synthetic construct
  source 1..24: mol_type = other DNA
catctttttccaacagggtaatga

SEQ ID NO: 619 - DNA, 24 bp, Synthetic construct
  source 1..24: mol_type = other DNA
tactttgttgaaaaccccaaataa

SEQ ID NO: 620 - DNA, 24 bp, Synthetic construct
  source 1..24: mol_type = other DNA
tatttggggttttcaacaaagtat

SEQ ID NO: 621 - DNA, 24 bp, Synthetic construct
  source 1..24: mol_type = other DNA
cttgaatttctgaacccacatcat

SEQ ID NO: 622 - DNA, 24 bp, Synthetic construct
  source 1..24: mol_type = other DNA
tgatgtgggttcagaaattcaagg

SEQ ID NO: 623 - DNA, 24 bp, Synthetic construct
  source 1..24: mol_type = other DNA
gttttgctgttaaagctagtacct

SEQ ID NO: 624 - DNA, 24 bp, Synthetic construct
  source 1..24: mol_type = other DNA
ggtactagctttaacagcaaaacc

SEQ ID NO: 625 - DNA, 24 bp, Synthetic construct
  source 1..24: mol_type = other DNA
ggttctaggaagttttgtgaaact

SEQ ID NO: 626 - DNA, 24 bp, Synthetic construct
  source 1..24: mol_type = other DNA
gtttcacaaaacttcctagaacca

SEQ ID NO: 627 - DNA, 24 bp, Synthetic construct
  source 1..24: mol_type = other DNA
tgggtagaagtgtttgtgttaagg

SEQ ID NO: 628 - DNA, 24 bp, Synthetic construct
  source 1..24: mol_type = other DNA
cttaacacaaacacttctacccaa

SEQ ID NO: 629 - DNA, 24 bp, Synthetic construct
  source 1..24: mol_type = other DNA
tagtagtagtgctcttttagagct

SEQ ID NO: 630 - DNA, 24 bp, Synthetic construct
  source 1..24: mol_type = other DNA
gctctaaaagagcactactactac

SEQ ID NO: 631 - DNA, 24 bp, Synthetic construct
  source 1..24: mol_type = other DNA
gtacctgagaccaaaaaggagaat

SEQ ID NO: 632 - DNA, 24 bp, Synthetic construct
  source 1..24: mol_type = other DNA
ttctcctttttggtctcaggtaca

SEQ ID NO: 633 - DNA, 24 bp, Synthetic construct
  source 1..24: mol_type = other DNA
ttgattttgagcttcctatgtatg

SEQ ID NO: 634 - DNA, 24 bp, Synthetic construct
  source 1..24: mol_type = other DNA
atacataggaagctcaaaatcaag

SEQ ID NO: 635 - DNA, 24 bp, Synthetic construct
  source 1..24: mol_type = other DNA
cttgctgtggttggtggtggccct

SEQ ID NO: 636 - DNA, 24 bp, Synthetic construct
  source 1..24: mol_type = other DNA
gggccaccaccaaccacagcaaga

SEQ ID NO: 637 - DNA, 24 bp, Synthetic construct
  source 1..24: mol_type = other DNA
caggacttgctgttgcacagcaag

SEQ ID NO: 638 - DNA, 24 bp, Synthetic construct
  source 1..24: mol_type = other DNA
ttgctgtgcaacagcaagtcctgc

SEQ ID NO: 639 - DNA, 24 bp, Synthetic construct
  source 1..24: mol_type = other DNA
ttctgaagcaggactctctgtttg

SEQ ID NO: 640 - DNA, 24 bp, Synthetic construct
  source 1..24: mol_type = other DNA
aaacagagagtcctgcttcagaaa

SEQ ID NO: 641 - DNA, 24 bp, Synthetic construct
  source 1..24: mol_type = other DNA
tcaattgatccgaatcctaaattg

SEQ ID NO: 642 - DNA, 24 bp, Synthetic construct
  source 1..24: mol_type = other DNA
aatttaggattcggatcaattgaa

SEQ ID NO: 643 - DNA, 24 bp, Synthetic construct
  source 1..24: mol_type = other DNA
tatggcctaataactatggtgttt

SEQ ID NO: 644 - DNA, 24 bp, Synthetic construct
  source 1..24: mol_type = other DNA
aacaccatagttattaggccatat

SEQ ID NO: 645 - DNA, 24 bp, Synthetic construct
  source 1..24: mol_type = other DNA
ttgttagattgtctagatgctacc

SEQ ID NO: 646 - DNA, 24 bp, Synthetic construct
  source 1..24: mol_type = other DNA
gtagcatctagacaatctaacaag

SEQ ID NO: 647 - DNA, 24 bp, Synthetic construct
  source 1..24: mol_type = other DNA
ggtctggtgcagcagtgtacattg

SEQ ID NO: 648 - DNA, 24 bp, Synthetic construct
  source 1..24: mol_type = other DNA
aatgtacactgctgcaccagacca

SEQ ID NO: 649 - DNA, 24 bp, Synthetic construct
  source 1..24: mol_type = other DNA
tgataatacggctaaagatcttca

SEQ ID NO: 650 - DNA, 24 bp, Synthetic construct
  source 1..24: mol_type = other DNA
gaagatctttagccgtattatcat

SEQ ID NO: 651 - DNA, 24 bp, Synthetic construct
  source 1..24: mol_type = other DNA
agaccttatggaagggttaaccgg

SEQ ID NO: 652 - DNA, 24 bp, Synthetic construct
  source 1..24: mol_type = other DNA
cggttaacccttccataaggtcta

SEQ ID NO: 653 - DNA, 24 bp, Synthetic construct
  source 1..24: mol_type = other DNA
aacagctgaaatcgaaaatgatgc

SEQ ID NO: 654 - DNA, 24 bp, Synthetic construct
  source 1..24: mol_type = other DNA
catcattttcgatttcagctgttt

SEQ ID NO: 655 - DNA, 24 bp, Synthetic construct
  source 1..24: mol_type = other DNA
ttccaccaagccaaagttataaag

SEQ ID NO: 656 - DNA, 24 bp, Synthetic construct
  source 1..24: mol_type = other DNA
tttataactttggcttggtggaat

SEQ ID NO: 657 - DNA, 24 bp, Synthetic construct
  source 1..24: mol_type = other DNA
tgattcatgaggaatcgaaatcca

SEQ ID NO: 658 - DNA, 24 bp, Synthetic construct
  source 1..24: mol_type = other DNA
ggatttcgattcctcatgaatcac

SEQ ID NO: 659 - DNA, 24 bp, Synthetic construct
  source 1..24: mol_type = other DNA
gttgatatgcaatgatggtattac

SEQ ID NO: 660 - DNA, 24 bp, Synthetic construct
  source 1..24: mol_type = other DNA
taataccatcattgcatatcaaca

SEQ ID NO: 661 - DNA, 24 bp, Synthetic construct
  source 1..24: mol_type = other DNA
attcaggcaacggtggtgctcgat

SEQ ID NO: 662 - DNA, 24 bp, Synthetic construct
  source 1..24: mol_type = other DNA
tcgagcaccaccgttgcctgaata

SEQ ID NO: 663 - DNA, 24 bp, Synthetic construct
  source 1..24: mol_type = other DNA
caactggcttctctagatctcttg

SEQ ID NO: 664 - DNA, 24 bp, Synthetic construct
  source 1..24: mol_type = other DNA
aagagatctagagaagccagttgc

SEQ ID NO: 665 - DNA, 24 bp, Synthetic construct
  source 1..24: mol_type = other DNA
tcaaaaacctcccacctaccgcgt

SEQ ID NO: 666 - DNA, 24 bp, Synthetic construct
  source 1..24: mol_type = other DNA
cgcggtaggtgggaggtttttgaa

SEQ ID NO: 667 - RNA, 24 nt, Synthetic construct
  source 1..24: mol_type = other RNA
tcaaaaacctcccacctaccgcgt

SEQ ID NO: 668 - RNA, 24 nt, Synthetic construct
  source 1..24: mol_type = other RNA
cgcggtaggtgggaggtttttgaa

SEQ ID NO: 669 - DNA, 24 bp, Synthetic construct
  source 1..24: mol_type = other DNA
tcaaaaacctcccacctaccgcgt

SEQ ID NO: 670 - DNA, 24 bp, Synthetic construct
  source 1..24: mol_type = other DNA
cgcggtaggtgggaggtttttgaa

SEQ ID NO: 671 - DNA, 24 bp, Synthetic construct
  source 1..24: mol_type = other DNA
tcaaaaacctcccacctaccgcgt

SEQ ID NO: 672 - DNA, 24 bp, Synthetic construct
  source 1..24: mol_type = other DNA
cgcggtaggtgggaggtttttgaa

SEQ ID NO: 673 - DNA, 24 bp, Synthetic construct
  source 1..24: mol_type = other DNA
tcaaaaacctcccacctaccgcgt

SEQ ID NO: 674 - DNA, 24 bp, Synthetic construct
  source 1..24: mol_type = other DNA
cgcggtaggtgggaggtttttgaa

(19) United States
(12) Patent Application Publication
(10) Pub. No.: US 2025/0092408 A1
(43) Pub. Date: Mar. 20, 2025

(54) PROGRAMMABLE EPIGENETIC CONTROL OF GENE EXPRESSION IN PLANTS

(71) Applicant: DECIBEL BIO, INC., EMERYVILLE, CA (US)

(72) Inventors: Travis BAYER, Emeryville, CA (US); Kevin L. SCHNEIDER, Emeryville, CA (US); Aden KINNE, Emeryville, CA (US); Jennifer Adele SAMSON, Emeryville, CA (US); Itxaso GARAY, Emeryville, CA (US)

(21) Appl. No.: 18/969,763

(22) Filed: Dec. 5, 2024

Related U.S. Application Data
(62) Division of application No. 18/169,601, filed on Feb. 15, 2023, now Pat. No. 12,203,083.
Application No. 18/169,601 is a continuation of application No. 17/476,097, filed on Sep. 15, 2021, now Pat. No. 11,591,608.
Application No. 17/476,097 is a continuation of application No. PCT/US2020/023391, filed on Mar. 18, 2020.
(60) Provisional application No. 62/820,172, filed on Mar. 18, 2019.

(51) Int. Cl. C12N 15/82

(52) U.S. Cl. CPC C12N 15/8218; C12N 15/825; C12N 15/8262; C12N 15/8297; C12N 15/8298

(57) ABSTRACT

Disclosed herein are artificially synthesized nucleic acid constructs to guide an epigenetic modification for at least partially silencing or activating a target gene in an organism such as a plant or seed, and formulations thereof. Also disclosed are methods of applying such nucleic acid constructs to the plant or to the seed. Also disclosed are engineered seeds and plants obtained by the epigenetic modification.

### CROSS-REFERENCE

The present application is a divisional of U.S. Nonprovisional application Ser. No. 18/169,601, filed Feb. 15, 2023, which is a continuation of U.S. Nonprovisional application Ser. No. 17/476,097, filed Sep. 15, 2021, which is a continuation of PCT/US2020/023391, filed Mar. 18, 2020, which claims the benefit of U.S. Provisional Application Ser. No. 62/820,172, filed Mar. 18, 2019, each of which are hereby incorporated by reference in their entirety. SEQUENCE LISTING

The instant application contains a Sequence Listing in XML format. The Sequence Listing, named 1370744.xml, was created on Feb. 15, 2023, is 583 Kilobytes in size, and is hereby incorporated by reference in its entirety.

### BRIEF SUMMARY

In some aspects, disclosed herein is a nucleic acid construct, e.g., a polynucleotide. In some instances, the present disclosure provides an artificial nucleic acid construct, wherein the artificial nucleic acid construct comprises: (a) a ribose modified at a 2′ or 3′ position, or a deoxyribose modified at a 2′ or 3′ position, or a combination thereof; and (b) a terminal end overhang, wherein: the artificial nucleic acid construct is double stranded; at least one strand of the artificial nucleic acid construct independently comprises a length from at least: about 10 to about 30 nucleotides or nucleosides or a combination thereof; and when contacted with an organism, at least a portion of the artificial nucleic acid construct is configured to facilitate: i) an epigenetic modification of at least one base of a nucleotide or nucleoside in a nucleic acid sequence of the organism, ii) a silencing of a target mRNA sequence that is at least partially complementary to the at least one strand of the artificial nucleic acid construct, iii) a cleavage of the target mRNA sequence, or iv) any combination of i), ii), or iii). In some instances, the artificial nucleic acid construct comprises no epigenetic modification on any purine or pyrimidine base of a nucleotide or nucleoside. In some instances, the artificial nucleic acid construct facilitates the epigenetic modification through a system that at least in part comprises a DNA methyltransferase, a biologically active fragment thereof, or a derivative thereof. In some instances, the artificial nucleic acid construct facilitates the epigenetic modification through a system that at least in part comprises a DNA acetyltransferase, a biologically active fragment thereof, or a derivative thereof. In some instances, the system comprises at least a portion of at least one component of an RNA directed DNA methylation pathway. In some instances, the at least one component comprises a protein or a portion thereof. In some instances, the protein or portion thereof is an enzyme or a portion thereof. In some instances, the artificial nucleic acid construct is capable of facilitating the epigenetic modification in the absence of a CRISPR, a CRISPR-associated protein (Cas), a biologically active fragment thereof, a derivative thereof, a fusion protein thereof, or any combination thereof. In some instances, the artificial nucleic acid construct is capable of facilitating the epigenetic modification in the absence of a Cas that comprises Cas1, Cas2, Cas3, Cas4, Cas5, Cas6, Cas7, Cas8, Cas9, or any combination thereof. In some instances, at least about 80% of sugars of the artificial nucleic acid construct, based on a total number of the sugars, each independently comprises a deoxyribose, modified deoxyribose, or a combination thereof. In some instances, less than about 20% of sugars of the artificial nucleic acid construct, based on a total number of the sugars, each independently comprises a ribose, a modified ribose, or a combination thereof. In some instances, the artificial nucleic acid construct is at least partially encoded by an RNA or DNA: nucleotide or nucleoside, or a combination thereof. In some instances, at least about 80% of the nucleotides, nucleosides, or a combination thereof of the artificial nucleic acid construct are DNAs. In some instances, a terminal nucleotide or terminal nucleoside of the artificial nucleic acid construct comprises an RNA: nucleotide or nucleoside, or a combination thereof. In some instances, multiple nucleotides in the nucleic acid sequence have the same epigenetic modification. In some instances, at least 10-12 nucleotides in the nucleic acid sequence have the same epigenetic modification. In some instances, the nucleic acid sequence comprises at least about 100 contiguous nucleotides. In some instances, the nucleic acid sequence comprises a CpG island. In some instances, the modified ribose or the modified deoxyribose is at a terminal nucleotide or nucleoside of the artificial nucleic acid construct. In some instances, the modified ribose or the modified deoxyribose comprises a 2′-O-R group. In some instances, R group is selected from the group consisting of: an alkyl, an aryl, a haloalkyl, an amino, a methyl, acetyl, and a halo. In some instances, the R group is the methyl. In some instances, the artificial nucleic acid construct further comprises a modification in at least one of a purine or pyrimidine base. In some instances, the modification comprises a plurality of modifications. In some instances, the modification increases stability of the artificial nucleic acid construct. In some instances, the modification increases uptake of the artificial nucleic acid construct by the organism. In some instances, the modification is substantially positioned at a 3′ end of the artificial nucleic acid construct. In some instances, the modification is substantially positioned at a 5′ end of the artificial nucleic acid construct. In some instances, the modification comprises a methyl group, a methoxy group, an ester group, a fluoro group, a phosphorthioate backbone, or any combination thereof. In some instances, the terminal end overhang is a 3′ end overhang. In some instances, the artificial nucleic acid construct comprises two 3′ end overhangs. In some instances, at least one strand of the artificial nucleic acid construct independently comprises a length from about 20 to about 30nucleotides or nucleosides or a combination thereof. In some instances, the artificial nucleic acid construct comprises at least one deoxyribonucleic acid. In some instances, the artificial nucleic acid construct comprises at least one ribonucleic acid. In some instances, the artificial nucleic acid construct comprises at least one strand that is not phosphorylated at its 5′ terminus. In some instances, the organism comprises a plant, a seed, a fruit, a leaf, a stalk, a root, a flower, an archaeon, a bacterium, a fungus, a virus, a virus-like particle, a protist, an alga, a nematode, a portion of any of these, or any combination thereof. In some instances, the artificial nucleic acid construct comprises at least about: 80%, 85%, 90%, 95%, or 98%; or about 100% sequence identity or at least 10 contiguous bases of one or more of SEQ ID NOs: 1-62. In some instances, the artificial nucleic acid construct comprises at least about: 80%, 85%, 90%, 95%, or 98%; or about 100% sequence identity or at least 10 contiguous bases of one or more of SEQ ID NOs: 63-194. In some instances, the artificial nucleic acid construct comprises at least about: 80%, 85%, 90%, 95%, or 98%; or about 100% sequence identity or at least 10 contiguous bases of one or more of SEQ ID NOs: 195-404. In some instances, the artificial nucleic acid construct comprises at least about: 80%, 85%, 90%, 95%, or 98%; or about 100% sequence identity or at least 10 contiguous bases of one or more of SEQ ID NOs: 405-584. In some instances, the artificial nucleic acid construct comprises at least about: 80%, 85%, 90%, 95%, or 98%; or about 100% sequence identity or at least 10 contiguous bases of one or more of SEQ ID NOs: 585-684. In some instances, the artificial nucleic acid construct comprises a peptide nucleic acid, a morpholino, a locked nucleic acid, a glycol nucleic acid, a threose nucleic acid, or any combination thereof positioned in the backbone of the artificial nucleic acid construct. In some instances, the epigenetic modification is comprised in a polynucleotide sequence at least partially encoding one or more proteins of Nuclear RNA polymerase D1 (NRPD1), NRPE1, NRPD2/NRPE2, NRPD4/NRPE4, NRPE5, NRPE9B, NRPB1, RNA-Dependent RNA Polymerase 2 (RDR2), DICER-Like 3 (DCL3), HUA Enhancer 1 (HEN1),

Argonaute 4 (AGO4), AGO6, AGO9, Classy 1 (CLSY1), Defective in RNA-Directed DNA Methylation 1 (DRD1), Defective in Meristem Silencing 3 (DMS3), RNA-Directed DNA Methylation 1 (RDM1), KOW Domain-Containing Transcription Factor 1 (KTF1), Involved in De Novo 2 (IDN2), IDN2 Paralogue1 (IDP1), IDP2, DMS4, Domains Rearranged Methyltransferase 2 (DRM2), SUVH2, SUVH9, SUVR2, Microrchidia 1 (MORC1), MORC6, Sawadee homeodomain homologue 1 (SHH1), Histone Deacetylase 6 (HDA6), Jumonji 14 (JMJ14), Lysine-specific Histone Demethylase 1 (LDL1), LDL2, Ubiquitin-specific Protease 26 (UBP26), Needed For RDR2-Independent DNA Methylation (NERD), Chromomethylase 2 (CMT2), CMT3, Methyltransferase 1 (MET1), SUVH4, Decreased DNA Methylation 1 (DDM1), biologically active fragment thereof, a regulatory element associated therewith, an intron associated therewith, or any combination thereof. In some instances, the epigenetic modification is catalyzed by an enzyme or a catalytically or biologically active fragment thereof. In some instances, the enzyme or catalytically or biologically active fragment thereof is endogenous to the organism. In some instances, the enzyme or catalytically or biologically active fragment thereof comprises a TET family enzyme or a catalytically or biologically active fragment thereof. In some instances, the enzyme or catalytically or biologically active fragment thereof comprises a methyltransferase or a catalytically or biologically active fragment thereof. In some instances, the methyltransferase comprises a chromomethyltransferase (CMT), a domain rearranged methyltransferase (DRM), or a combination thereof. In some instances, the epigenetic modification adds a chemical group to the at least one base. In some instances, the epigenetic modification adds a methyl group to the at least one base, and wherein the methyl group is oxidized to a methoxy, formyl, or carboxyl group, or a carboxylic acid. In some instances, the at least one base is a cytosine. In some instances, the epigenetic modification comprises a methyl group, a hydroxymethyl group, a formyl group, a carboxyl group, or any combination thereof. In some instances, the epigenetic modification comprises the methyl group. In some instances, the nucleic acid sequence comprises a transcription regulatory region. In some instances, a strand of the double stranded artificial nucleic acid construct comprises at least about: 80%, 85%, 90%, 95%, or 98%; or about 100% sequence identity to a portion of a transcription regulatory region. In some instances, the transcription regulatory region comprises at least about 30% guanine cytosine (GC) content.

In some aspects, the present disclosure provides a plurality of artificial nucleic acid constructs disclosed herein (double stranded or single stranded), for example at least about: 2-100, 2-90, 2-80, 2-70, 2-60, 2-50, 2-40, 2-30, 2-24, 2-12, 4-100, 4-90, 4-80, 4-70, 4-60, 4-50, 4-40, 4-30, 4-24, 4-12, 6-100, 6-90, 6-80, 6-70, 6-60, 2-50, 6-40, 6-30, 6-24, 6-12, 8-100, 8-90, 8-80, 8-70, 8-60,8-50, 8-40, 8-30, 8-24, 8-12, 10-100, 10-90, 10-80, 10-70, 10-60, 10-50, 10-40, 10-30, 10-24, or 10-12 of the artificial nucleic acid constructs herein. In some instances, at least about 10% to about 100% of the number of the artificial nucleic acid constructs are different in the plurality. In some instances, each artificial nucleic acid construct in the plurality is different.

In some aspects, the present disclosure provides a method for making the artificial nucleic acid construct disclosed herein, comprising adding to the artificial nucleic acid construct a nucleotide or nucleoside comprising a ribose modified at a 2′ or 3′ position, or a deoxyribose modified at a 2′ or 3′ position, or a combination thereof.

In some aspects, the present disclosure provides a method for making the artificial nucleic acid constructs disclosed herein, comprising modifying a ribose, deoxyribose, or a combination thereof at the 2′ or 3′ position in the artificial nucleic acid construct.

In some aspects, the present disclosure provides a method for making the plurality of artificial nucleic acid constructs, comprising mixing two or more of the artificial nucleic acid constructs disclosed herein.

In some aspects, the present disclosure provides an isolated cell comprising the artificial nucleic acid construct disclosed herein or a plurality thereof. In some instances, the cell is a eukaryotic cell.

In some aspects, the present disclosure provides a plant, seed, solution, microorganism, fertilizer, or soil comprising the artificial nucleic acid construct disclosed herein or a plurality thereof.

In some aspects, the present disclosure provides a composition comprising: (a) an artificial nucleic acid construct disclosed herein and (b) an enzyme or a catalytically or biologically active fragment thereof that performs an epigenetic modification of at least one base in a nucleic acid sequence of an organism.

In some aspects, the present disclosure provides a composition comprising: a vector comprising one or more artificial nucleic acid constructs herein or a plurality thereof.

In some aspects, the present disclosure provides a kit comprising: (a) an artificial nucleic acid construct, a plurality thereof, or a composition thereof as disclosed herein; and (b) a container comprising a soil, a fertilizer, a seed, a plant, a liquid, or any combination thereof.

In some aspects, the present disclosure provides a formulation comprising: (a) an artificial nucleic acid construct, a plurality thereof, or a composition thereof as disclosed herein; and (b) an excipient. In some instances, the excipient is an agriculturally acceptable excipient. In some instances, the formulation further comprises a fertilizer or a soil.

In some aspects, the present disclosure provides a method of making a formulation, comprising contacting an artificial nucleic acid construct, a plurality thereof, or a composition thereof as disclosed herein with an excipient to form the formulation.

In some aspects, the present disclosure provides an engineered plant or seed comprising a heritable modification that at least partially silences or activates at least one gene of the engineered plant or seed, wherein the heritable modification comprises a methylated base in a transcription regulatory region of the at least one gene, and wherein the heritable modification does not comprise a transgene, optionally comprising the artificial nucleic acid construct or a plurality thereof. In some instances, two genes are silenced. In some instances, the at least one methylated base is not naturally methylated in the nucleic acid sequence. In some instances, the transcription regulatory region comprises at least one selected from the group consisting of: a transcription start site, a TATA box, and an upstream activating sequence. In some instances, the methylated base comprises a plurality of methylated bases.

In some aspects, the present disclosure provides a plurality of engineered plants or seeds comprising variable gene expressions that at least partially silence or activate at least one gene of the engineered plants or seeds, optionally comprising the artificial nucleic acid construct or a plurality thereof disclosed herein. In some instances, the engineered plants or seeds comprise at least one modified base in at least one gene therein, which is not naturally methylated in the gene. In some instances, the base is modified with an alkyl, an aryl, a haloalkyl, an amino, a methyl, acetyl, a halo, or a combination thereof. In some instances, the engineered plants or seeds comprise at least one methylated base in at least one gene therein. In some instances, the plurality comprises at least about: 10-20, 20-50, 50-100, 100-1000, 100-900, 100-800, 100-700, 100-600, 100-500, 100-400, 100-300, 100-240, 100-120, or 1000-10000 of engineered plants or seeds.

In some aspects, the present disclosure provides a method, comprising applying a substance to an agricultural object, wherein the agricultural object comprises a seed, a plant, a constituent of a plant, or any combination thereof, and wherein the substance comprises the artificial nucleic acid construct or a plurality thereof disclosed herein. In some instances, the method at least partially silencing or activating a gene in the agricultural object. In some instances, the gene is at least partially silenced for at least one or two reproduction cycles. In some instances, the applying results in one or more of the following: (a) preventing or reducing or delaying an enzymatic browning of the agricultural object; (b) increasing a growth rate, a yield, or a lifespan of the agricultural object; (c) decreasing a growth rate, a yield, or a lifespan of the agricultural object; (d) increasing a pest resistance, a salt tolerance, a heat tolerance, a heavy metal tolerance, a disease tolerance, or a drought resistance of the agricultural object; (e) increasing or at least partially decreasing an amount or a production of a molecule made by the agricultural object; (f) altering a color of at least a portion of the agricultural object; (g) increasing or at least partially decreasing a flowering rate of the agricultural object; (h) increasing a volume or a weight of the agricultural object; (i) improving a flavor or a texture of an edible product of the agricultural object; (j) increasing a shelf life of the agriculture product; (k) decreasing the number and size of seeds of the agriculture product; and (j) increasing a nutritional content of the agricultural object; when the agricultural object is compared to a comparable agricultural object without application of the substance comprising the artificial nucleic acid construct. In some instances, the agricultural object is a plant embryo. In some instances, the agricultural object is selected from the group consisting of: soybean, corn, rice, tomato, alfalfa, wheat, potato, and green algae. In some instances, the contacting decreases the growth rate, the yield, or the lifespan of a weed. In some instances, the contacting increases the growth rate, the yield, or the lifespan of the agricultural object, and wherein the molecule is a psychoactive substance. In some instances, the psychoactive substance comprises a tetrahydrocannabinol. In some instances, the substance is a liquid, soil, microorganism, fertilizer, or herbicide.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative instances, in which the principles of the disclosure are utilized, and the accompanying drawings of which:

FIG. 1 depicts schematically treatment of an untreated seed with a plurality of CATS oligonucleotides.

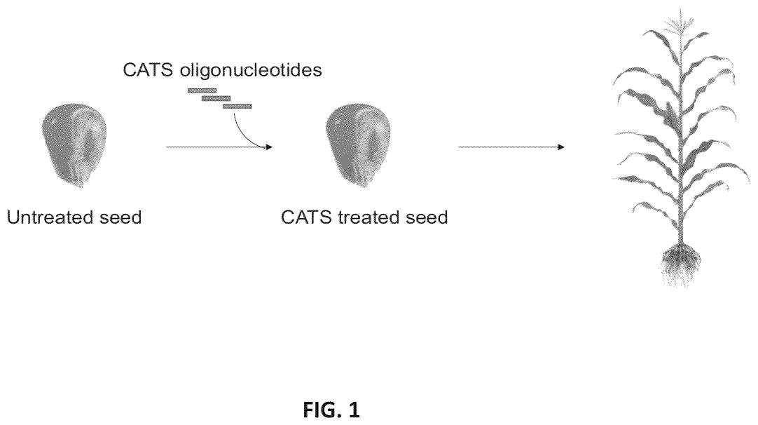

FIG. 2 depicts a non-limiting example of the design of a CATS oligonucleotide.

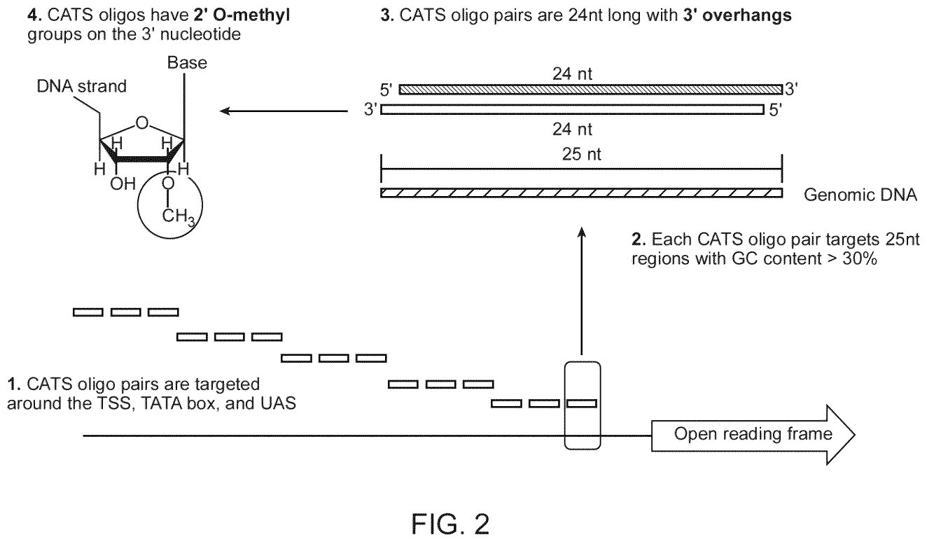

FIG. 3 depicts the result of silencing a PDS1 gene in maize by treatment with CATS oligonucleotides. PDS1-CATS plants show a pale green leaf phenotype (upper leaf), as compared to a control maize plant (lower leaf).

FIG. 4 depicts the results of silencing a PDS1 gene in maize plants by treatment with CATS oligonucleotides using an infiltration administration method.

FIG. 5 depicts bisulfite sequencing analysis of a PDS1 gene in CATS-treated maize plants. Treatment with CATS oligonucleotides results in methylated cytosines upstream of the PDS1 open reading frame.

FIGS. 6A-6D depict the result of silencing a LZY1 gene in maize by treatment with CATS oligonucleotides. FIG. 6A illustrates that in a wild type plant, shoots grow in the opposite direction of the force of gravity (gravitropism). FIG. 6B illustrates that LZY1-CATS plants show a loss of gravitropism compared to wild type plants. FIG. 6C illustrates that in a wild type plant, shoots grow in the opposite direction of the force of gravity (gravitropism). FIG. 6D illustrates that LZY1-CATS plants show a loss of gravitropism compared to wild type plants.

FIGS. 7A-7C depict the result of silencing a polyphenol oxidase (PPO) gene in potato plants. FIG. 7A illustrates that a CATS-treated potato shows a slower enzymatic browning as compared to an untreated control potato. FIG. 7B depicts RT-PCT analysis of PPO mRNA levels in CATS-treated potato plants. CATS plants have reduced levels of the mRNA transcript, as compared to the control mRNA in untreated plants (FIG. 7C).

FIG. 8 depicts the result of silencing BWF1 and BR2 genes in maize. Treatment with CATS oligonucleotides targeting BWF1 and BR2 results in shorter maize plants compared to an untreated control plant.

FIG. 9 depicts using epigenetic methods to create variable populations compared to wildtype populations. The population treated with the epigenetic method can have decreased gene expression compared to the wildtype population.

FIG. 10 depicts using CATS oligonucleotides to target the old gold gene in the tomato plant. The 1 kb treatment can decrease the gene expression in individual plants compared to the RO control.

FIG. 11 depicts exemplary CATS oligonucleotides with modified backbones comprising phosphorothioate modifications, 2′O-Methyl modifications, 2′-Fluoro modifications, or any combination thereof.

### DETAILED DESCRIPTION

### Introduction

Disclosed herein are molecular techniques for at least partially silencing or activating specific target genes in plants to elicit desired phenotypes with commercial value. In some instances, this technology is referred to as Coat Applied Transcriptional Silencing (CATS) and uses specifically engineered DNA oligonucleotides constructs to induce nitrogenous base modification (e.g., cytosine methylation) and epigenetic silencing of target genes. The methods disclosed herein allow the specific and multiplex manipulation of plant gene expression, with design and build workflow of days, compared to the months or years necessary for transgenic approaches.

An engineered DNA oligonucleotide described herein can be prepared to mimic a nucleic acid sequence present in an RNA interference pathway. In some cases, an RNA interference pathway can be a pathway naturally present in an organism (e.g. a plant, a fungus, a bacterium, or an animal). For example, an engineered DNA oligonucleotide can be prepared to mimic a nucleic acid sequence present in a Drosophila melanogaster, Caenorhabditis elegans, or Arabidopsis thaliana RNA interference pathway. An exemplary RNA interference pathway can include an Arabidopsis thaliana RNA interference pathway. In the exemplary Arabidopsis thaliana RNA interference pathway, a first RNA nucleic acid is prepared through transcription using a polymerase IV enzyme. The first RNA nucleic acid transcribed from polymerase IV can be single stranded, or can be converted into a double stranded RNA nucleic acid. A resulting double stranded nucleic acid can be processed by a dicer enzyme (for instance, a Dicer-Like 3 (DCL3) enzyme) to produce smaller double stranded RNA nucleic acid fragments. Such RNA nucleic acid fragments can be methylated on a 2′ or 3′ hydroxyl group present on a ribose sugar present on the RNA nucleic acid fragments by a HUA Enhancer 1 (HEN1) enzyme or biological equivalent thereof. The methylated double-stranded RNA strand can be processed by an Argonaute 4 (AGO4) enzyme or biological equivalent thereof to produce a single stranded, methylated RNA nucleic acid. In some cases, the methylated RNA nucleic acid can modulate an expression of a gene (including a level of mRNA encoded by a gene or a polypeptide encoded by an mRNA) in an organism through multiple pathways. For instance, a methylated RNA nucleic acid can bind directly to an mRNA sequence encoding the polypeptide, thus acting as a silencing RNA nucleic acid. Further, a methylated RNA nucleic acid can recruit enzymes, such as AGO4, capable of inducing a cleavage of the mRNA when the methylated RNA nucleic acid associates with the mRNA sequence. Further, a methylated mRNA nucleic acid can associate with a DNA methylating enzyme, such as Domains Rearranged Methyltransferase 2 (DRM2), to catalyze a de novo methylation of genomic DNA encoding the mRNA sequence. In some cases, a methylated RNA nucleic acid can modulate an expression of a gene in an organism through any one, any 2, or all of 3 of these actions.

Accordingly, an engineered oligonucleotide can be engineered to mimic a nucleic acid present in an RNA interference pathway. For example, an engineered oligonucleotide can be engineered to mimic a methylated RNA nucleic acid, such as an RNA nucleic acid methylated using a HEN1 enzyme. Accordingly, such an engineered oligonucleotide can modulate an expression of a gene (including a level of mRNA encoded by a gene or a polypeptide encoded by an mRNA) in an organism through any combination of the actions referenced above. For example, an engineered oligonucleotide can bind directly to an mRNA sequence encoding the polypeptide, thus acting as a silencing nucleic acid. Further, an engineered oligonucleotide can recruit enzymes, such as AGO4,capable of inducing a cleavage of the mRNA when the engineered oligonucleotide associates with the mRNA sequence. Further, an engineered oligonucleotide can associate with a DNA methylating enzyme, such as Domains Rearranged Methyltransferase 2 (DRM2), to catalyze a de novo methylation of genomic DNA encoding the mRNA sequence. In some cases, an engineered oligonucleotide can modulate an expression of a gene in an organism through any one, any 2, or all of 3 of these actions.

Furthermore, an engineered oligonucleotide can be designed to be at least partially complementary to a desired target mRNA sequence. In some cases, a desired target mRNA sequence can be an mRNA that is a target of a natural methylated RNA nucleic acid in an RNA interference pathway. In some cases, a desired target mRNA can be an mRNA that is separate and distinct from any target of a natural methylated RNA nucleic acid in an RNA interference pathway. Accordingly, highly specific gene regulation can be accomplished by generating engineered oligonucleotides that are capable of associating with a target mRNA nucleic acid of interest.

In some instances, engineered nucleic acid constructs are designed with high sequence homology to a transcription regulatory region upstream of a target gene's open reading frame. In some instances, the nucleic acid constructs can be a double stranded DNA construct having at least one end overhang, such that the DNA construct mimics the double stranded RNA recognized by the plant's endogenous DNA methylation machinery, thereby directing endogenous DNA methylation to the transcription regulatory region. In some instances, methylation of cytosines in a gene's transcription regulatory region at least partially silence the gene.

Use of the nucleic acid constructs described herein has several advantages. The specifically designed oligonucleotides introduce cytosine methylation at a defined region of a plant genome, which can control transcript expression levels. The nucleic acid constructs can be introduced by application to the seed coat or to the growing roots of the plant, enabling rapid construction of plants with tailored gene expression profiles. In some instances, the modified bases (e.g., methylated cytosines) are heritable, enabling generation of parental breeding lines with desired expression profiles. Methylation and altered transcript levels can be propagated through future progeny generations. Additionally, the application of nucleic acid constructs to plants can be multiplexed by application of mixtures of nucleic acid constructs having unique targeting sequences, such that multiple transcription regulatory regions of a gene, and/or multiple gene, may be targeted simultaneously.

Yield drivers can be regulated by epigenetic mechanisms. Examples of yield drivers are hybrid vigor, photosynthesis and seed size. Stress resistance can be regulated by epigenetic mechanisms. Examples of stress resistance are disease resistance, drought tolerance, salt tolerance, heat tolerance and heavy metal stress. The applications of the CATs nucleotides can be broad and potentially encompass many gene regulatory networks that affect phenotype.

For example, constructs as described herein may provide one or more epigenetic modifications to one or more genes of an organism-such as an agricultural product. Introduction of such epigenetic modifications may modify one or more characteristics of the agricultural product. Such characteristics may include one or more of: photosynthesis, heterosis, nutrient efficiency, energy efficiency, seed size, plant biomass, circadian rhythm, flowering time, seed development, root development, disease resistance, drought tolerance, salt tolerance, heat tolerance, heavy metal stress, or any combination thereof. One or more characteristics may comprise flavor, color, texture, shelf-life, seedless varieties, or any combination thereof. An epigenetic modification to a gene may modify a characteristic. An epigenetic modification to a gene may modify a plurality of characteristics. A plurality of epigenetic modifications may modify a characteristic. A plurality of epigenetic modifications may modify a plurality of characteristics.

Additionally, constructs as described herein can be utilized to create diverse pools of organisms such as an agricultural product. Constructs can introduce one or more epigenetic modifications into one or more genes of an organism. Introduction of one or more epigenetic modifications can result in at least partial silencing or at least partial activation of one or more genes. Activation or silencing or a combination thereof of one or more genes in an organism can result in diversifying a species. Introducing epigenetic modifications into one or more genes of an organism can create a diverse pool of progeny. Such downstream applications can include breeding selection, such as selecting for advantageous characteristics.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the formulations or unit doses herein, some methods and materials are now described. Unless mentioned otherwise, the techniques employed or contemplated herein are standard methodologies. The materials, methods and examples are illustrative only and not limiting.

The details of one or more inventive instances or aspects are set forth in the accompanying drawings, the claims, and the description herein. Other features, objects, and advantages of the inventive instances or aspects disclosed and contemplated herein can be combined with any other instance or aspect unless explicitly excluded.

The open terms for example “contain,” “containing,” “include,” “including,” and the like mean comprising.

The singular forms “a”, “an”, and “the” are used herein to include plural references unless the context clearly dictates otherwise.

Unless otherwise indicated, some instances herein contemplate numerical ranges. When a numerical range is provided, unless otherwise indicated, the range can include the range endpoints. Unless otherwise indicated, numerical ranges can include all values and subranges therein as if explicitly written out.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

The term “facilitates” herein can refer to i) a machinery that when a construct is introduced to an organism with an appropriate molecular system for an epigenetic modification, it facilitates or enables the organism to use at least a part of the molecular system to epigenetically modify a nucleic acid sequence in the organism, or ii) a silencing of a target mRNA sequence that is at least partially complementary to the at least one strand of the artificial nucleic acid construct, iii) a cleavage of the target mRNA sequence, or iv) any combination of i), ii), or iii).

The term “compounds” can refer to compounds encompassed by generic formulae disclosed herein, any subgenus of those generic formulae, and any specific compounds within those generic or subgeneric formulae. The compounds can be a specific species, a subgenus or larger genus identified either by their chemical structure and/or chemical name. Further, compounds also include substitutions or modifications of any of such species, subgenuses or genuses, which are set forth herein. When the chemical structure and chemical name conflict, the chemical structure can be determinative of the identity of the compound. The compounds can contain one or more chiral centers and/or double bonds and therefore, can exist as stereoisomers, isomers, enantiomers or diastereomers. Accordingly, the chemical structures within the scope of the specification encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Further, when partial structures of the compounds are illustrated, asterisks indicate the point of attachment of the partial structure to the rest of the molecule. Enantiomeric and stereoisomeric mixtures can be resolved into their component enantiomers or stereoisomers using separation techniques or chiral synthesis techniques well known to the skilled artisan. The compounds can include any salt or solvate forms of the compounds. The compounds can include any derivatives of the compounds.

The term “derivative,” which can be used interchangeably with the term “analog.” Compound A can be a derivative or analog of compound B if 1, 2, 3, 4, or 5 atoms of compound A is replaced by another atom or a functional group (e.g., amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl) to form compound B. The term “derivative” may also refer to a chemical compound that is structurally similar to another but differs slightly in composition (as in the replacement of one atom by an atom of a different element or in the presence of a particular functional group)

The term “solvate” can include, but is not limited to, a solvate that retains one or more of the activities and/or properties of the compound and that is not undesirable. Examples of solvates include, but are not limited to, a compound in combination with water, isopropanol, ethanol, methanol, DMSO, ethyl acetate, acetic acid, ethanolamine, or combinations thereof.

The term “salt” can include, but are not limited to, salts that retain one or more of the activities and properties of the free acids and bases and that are not undesirable. Illustrative examples of salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methanesulfonates, propanesulfonates, naphthalene-1-sulfonates, naphthalene-2-sulfonates, and mandelates.

Unless otherwise indicated, a chemical structure can refer to any compound having the chemical structure.

Unless otherwise indicated, formulations herein can be powdery, pellets, or beads or be a liquid.

Unless otherwise indicated, formulations herein can contain water in an amount from about 0% to about 15% w/w, for example about: 0-10%, 0-5%, or 0-1% w/w; or less than about: 1%, 2%, 3%, 4%, 5%, 6%, 7%, 8%, 9%, 10%, 11%, 12%, 13%, 14%, 15%, 20%, 25%, 30%, 35%, 40%, 45%, 50%, 55%, 60%, 65%, 70%, 75%, 80%, 85%, 90% or 99% w/w, based on the weight of the formulation.

Unless otherwise indicated, whenever there is a stereocenter in a structure disclosed or illustrated herein, the stereocenter can be R or S in each case.

Unless otherwise indicated, whenever there is a symbol

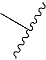

when used as part of a molecular structure herein can refer to a single bond.

The term “amino” can refer to functional groups that contain a basic nitrogen atom with a lone pair. For example, amino can include the radical

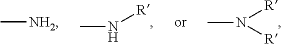

wherein each R′ is independently H, halo, alkyl, aryl, heteroalkyl, arylalkyl, heteroaryl, heteroarylalkyl, cycloalkyl, or heterocycloalkyl.

The term “halo” or “halogen” can refer to fluorine, chlorine, bromine or iodine or a radical thereof.

The term “alkyl” can refer to a saturated or unsaturated, branched, straight-chain or cyclic monovalent hydrocarbon group derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl; and the like.

The term “aryl” can refer to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain instances, an aryl group comprises from 6 to 20 carbon atoms.

The terms “heteroalkyl, heteroalkanyl, heteroalkenyl, heteroalkynyl” refer to alkyl, alkanyl, alkenyl and alkynyl groups, respectively, in which one or more of the carbon atoms (and any associated hydrogen atoms) are each independently replaced with the same or different heteroatomic groups. Typical heteroatomic groups include, but are not limited to, —O—, —S—, —O—O′, —S—S—, —O—S—, —NR′—, ═N—N═, —N═N—, —N═N—NR′—, —PH—, —P(O)₂—, —O—P(O)₂—, —S(O)—, —S(O)₂—, —SnH₂— and the like, wherein R′ is hydrogen, alkyl, substituted alkyl, cycloalkyl, substituted cycloalkyl, aryl or substituted aryl.

The term “heteroaryl” can refer to a monovalent heteroaromatic group derived by the removal of one hydrogen atom from a single atom of a parent heteroaromatic ring system. Typical heteroaryl groups include, but are not limited to, groups derived from acridine, arsindole, carbazole, ß-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain instances, the heteroaryl group is from 5-20 membered heteroaryl, and in other instances is from 5-10 membered heteroaryl. In certain instances heteroaryl groups are those derived from thiophene, pyrrole, benzothiophene, benzofuran, indole, pyridine, quinoline, imidazole, oxazole and pyrazine.

The term “arylalkyl” can refer to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain instances, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀).

The term “heteroarylalkyl” can refer to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heteroarylalkynyl is used. In certain instances, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl.

The term “cycloalkyl” can refer to a saturated or unsaturated cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In a certain instance, the cycloalkyl group is (C₃-C₁₀) cycloalkyl, or in certain instances (C₃-C₆) cycloalkyl.

The term “heterocycloalkyl” can refer to a saturated or unsaturated cyclic alkyl group in which one or more carbon atoms (and any associated hydrogen atoms) are independently replaced with the same or different heteroatom. Typical heteroatoms to replace the carbon atom(s) include, but are not limited to, N, P, O, S, and Si. Typical heterocycloalkyl groups include, but are not limited to, groups derived from epoxides, imidazolidine, morpholine, piperazine, piperidine, pyrazolidine, pyrrolidine, quinuclidine, and the like.

The term “diastereomeric excess” (DE) can refer to the difference from the relative abundance of two diastereomers. For instance, if there are two diastereomers and their mole or weight percentages are A and B, then DE can be calculated as: DE=[(A−B)/(A+B)]*100%. For example, if a mixture contains 75% of one diastereomer and 25% of the other diastereomer, the diastereomeric excess is 50%. In another example, if a mixture that is 95% of one diastereomer, the diastereomeric excess is 90%.

The term “enantiomeric excess” (EE) can refer to the difference from the relative abundance of two enantiomers. For instance, if there are two enantiomers and their mole or weight percentages are A and B, then EE can be calculated as: EE=[(A−B)/(A+B)]*100%. For example, if a mixture contains 75% of one enantiomer and 25% of the other enantiomer, the enantiomeric excess is 50%. In another example, if a mixture that is 95% of one enantiomer, the enantiomeric excess is 90%.

The term “substituted” can refer to a group in which one or more hydrogen atoms are each independently replaced with the same or different substituent(s). Typical substituents include, but are not limited to halo, alkyl, aryl, heteroalkyl, arylalkyl, heteroaryl, heteroarylalkyl, cycloalkyl, and heterocycloalkyl.

Unless otherwise indicated, “treated” can refer to “contacted.” Similarly, “untreated” can refer to “uncontacted.”

The term “substantially comparable plant” can refer to a plant of the same species as an earlier referenced plant. For example, a substantially comparable but otherwise uncontacted plant belongs to the same species as a contacted plant. The substantially comparable but otherwise uncontacted plant can have a height of about 80% to 120% of the contacted plant (as measured from the surrounding soil to the highest point of the plant) and/or can have a mass of about 80% to 120% of the contacted plant.

The term “drought” can mean conditions with less than 20 inches, 15 inches, 10 inches, or 5 inches of rainfall within the past 12 months. The term “drought” can also mean conditions with a Palmer Drought Severity Index (PDSI) of less than −1.0. The term “adequately irrigated condition” can mean a condition with more than 20 inches of rainfall within the past 12 months. The term “adequately irrigated condition” can mean a condition with a PDSI of more than −1.0.

The term “plant” can be used interchangeably with the term “crop” and can include, but is not limited to any crop, cultivated plant, fungus, or alga that may be harvested for food, clothing, livestock fodder, biofuel, medicine, or other uses. For example, plants include field and greenhouse crops, including but not limited to broad acre crops, fruits and vegetables, perennial tree crops, and ornamentals. Plants include, but are not limited to sugarcane, pumpkin, maize (corn), wheat, rice, cassava, soybeans, hay, potatoes, cotton, tomato, alfalfa, and green algae. Plants also include, but are not limited to any vegetable, such as cabbage, turnip, carrot, parsnip, beetroot, lettuce, beans, broad beans, peas, potato, eggplant, tomato, cucumber, pumpkin, squash, onion, garlic, leek, pepper, spinach, yam, sweet potato, and cassava. In some instances, the plant can also include a fruit, a leaf, a stalk, a root, a flower, a plant embryo, or any combination thereof.

### Nucleic Acid Constructs

Disclosed herein are nucleic acid constructs for at least partially silencing or activating a gene in an organism, wherein the nucleic acid construct is configured to guide endogenous modification (e.g., methylation) of at least one base of the gene in the organism. In some instances, the nucleic acid construct can be single-stranded or double-stranded.

In some instances, the nucleic acid construct can be an artificial double-stranded nucleic acid construct comprising 1) a modified ribose or a modified deoxyribose or a combination thereof and 2) a terminal end overhang, wherein, when the nucleic acid construct associates with a nucleic acid sequence of an organism, an enzyme performs an epigenetic modification of at least one base in the nucleic acid sequence of the organism. In some instances, the modified ribose or the modified deoxyribose is comprised in a terminal nucleotide of the nucleic acid construct. In some instances, the nucleic acid construct associates with an argonaut protein that associates with the nucleic acid sequence.

In some instances, the nucleic acid construct can comprise deoxyribonucleic acid (DNA), ribonucleic acid (RNA), or a combination thereof. In some instances, the nucleic acid construct can comprise at least one deoxyribonucleic acid. In some instances, the nucleic acid construct can comprise at least about: 2, 3, 4, 5, 6, 7, 8, 9, 10, 15, 20, 25, 30, 35, 40, 45, 50, 55, 60, 65, 70, 75, 80, 85, or 100 deoxyribonucleic acids. In some instances, the nucleic acid construct can comprise at least one ribonucleic acid. In some instances, the nucleic acid construct can comprise at least about: 2, 3, 4, 5, 6, 7, 8, 9, 10, 15, 20, 25, 30, 35, 40, 45, 50, 55, 60, 65, 70, 75, 80, 85, or 100 ribonucleic acids.

In some instances, the base of the gene may be a cytosine. In some instances, the base of the gene may be an adenine, a guanine, or a thymine.

In some instances, the nucleic acid construct may be a double stranded DNA construct. In some instances, the double stranded DNA construct may comprise two polynucleotide strands of the same length. In some instances, the double stranded DNA construct may comprise two polynucleotide strands of a different length. In some instances, the double stranded DNA construct may comprise at least one end overhang. In some instances, an end overhang may comprise a single nucleotide. In some instances, an end overhang may comprise at least about: 2, 3, 4, 5, 6, 7, 8, 9, or 10 nucleotides. In some instances, the double stranded DNA construct may comprise an end overhang at each end of the DNA construct. In some instances, the double stranded DNA construct may comprise an end overhang at each end of the DNA construct, wherein the end overhangs are of the same length. In some instances, the double stranded DNA construct may comprise a one-nucleotide end overhang at each end of the DNA construct. In some instances, the double stranded DNA construct may comprise a 3′-end overhang. In some instances, the double stranded DNA construct may comprise a 5′-end overhang. In some instances, the double stranded DNA construct may comprise a one-nucleotide 3′-end overhang at each end of the DNA construct.

In some instances, the nucleic acid construct may comprise at least one modified sugar, for example modified at the 2′ position and/or the 3′ position. Numbering of a nucleotide sugar should be understood to follow normal conventions of positional numbering in the art. Specifically, carbon numbering in nucleotide sugar is as illustrated by ribose sugar shown below:

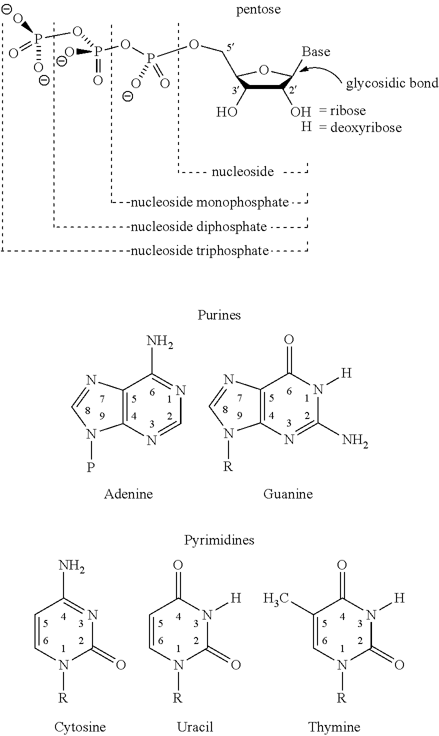

In some instances, the modified sugar may comprise a 2′-R, 2′-O—R, 3′-R, or 3′-O—R group. In some instances, the R group may be selected from the group consisting of alkyl, aryl, haloalkyl, amino, and halogen. In some instances, the R group can be a fluro-(F). In some instances, the R group can be methoxyethyl. In some instances, the R group can be methyl. In some instances, R can be —(C—O) n-R1, or the ribose modification comprises the structure represented by:

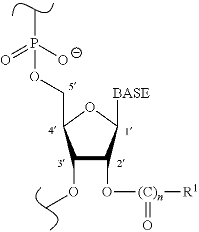

In some instances, R1 can be alkyl, alkenyl, alkynyl aryl, substituted alkyl, substituted alkenyl, substituted alkynyl, or a substituted aryl.

In some instances, the modified sugar may be comprised in a terminal nucleotide of at least one strand of the nucleic acid construct. In some instances, the modified sugar may be comprised in a 3′-terminal nucleotide of at least one strand of the nucleic acid construct. In some instances, the nucleic acid construct may be a double stranded DNA construct, wherein each strand of the DNA construct comprises at least one modified sugar. In some instances, the nucleic acid construct may be a double stranded DNA construct, wherein each strand of the DNA construct comprises at least one modified sugar in the strand's 3′-terminal nucleotide.

In some cases, the sugar can be a derivative of ribose. In some instances, the sugar can be arabinose. In some instances, the sugar may be 2′-deoxy-2′-fluoro-arabino (FANA). In some instances, the sugar may be a derivative of hexose (e.g. HNA; hexose nucleic acid). In some instances, the sugar may be a derivative of threose (e.g. TNA; threose nucleic acid). In some instances, the sugar may be replaced with a morpholino group and the backbone composed of phosphoramidate linkages (e.g. PMO; Phosphorodiamidate Morpholino Oligomer) In some instances, the sugar may be a bridged sugar (e.g. BNA; bridged nucleic acid). In some instances, the bridging carbon between the 2′ and 4′ positions may be a methylene group (e.g. LNA; locked nucleic acid). In some instances, the bridging carbon between the 2′ and 4′ positions may be an ethyl group. In some instances, the 2′,4′-constrained ethyl sugar derivative may be in the S stereochemical configuration (e.g. (S)-cET). In some instances, the sugar can be acyclic. In some instances, sugar may be a derivative or ribose lacking the 2′ and 3′-bond (e.g. UNA; unlocked nucleic acid). In some instances, the sugar may be a derivative of threoninol (e.g aTNA; threoninol nucleic acid). In some instances, the sugar may be a derivative of serinol (e.g. SNA; serinol nucleic acid). In some instances, the sugar may be a derivative of glycol (e.g. GNA; glycol nucleic acid).

In some cases, the sugars within each strand are linked 3′ to 5′ by a bridging phosphodiester linkage. In some instances one oxygen within the phosphodiester linkage may be replaced with a sulfur forming a phosphorthioate. In some instances, two oxygens within phosphodiester linkage are replaced with sulfur forming a phosphorodithioate linkage. In some instances, the 3′-5′ phosphodiester contains an additional ester group on a non-bridging oxygen. In some instances the sugar bridging phosphate may be a phosphotriester. In some instances, the phosphotriester may be stable. In some instances, the phosphotriester may be bioreversible.

In some cases, a nucleic acid disclosed herein is associated with or encapsulated within a liposome. In some instances, the liposome may be comprised of a cationic peptide (e.g. DOTAP). In some instances, the liposome may be comprised of an ionizable lipid (e.g. DLinDMA or KC2-DMA).

In some cases, a nucleic acid disclosed herein may be associated with or encapsulated within a nano or microparticle. In some instances, nano or microparticle may be comprised of poly lactic-co-glycolic acid (PLGA). In some instances, the nucleic acid may be attached to a nanoparticle. In some instances the nanoparticle may be a liposome. In some instances the nanoparticle may be a micelle. In some instances the nanoparticle comprises silica. In some instances the nanoparticle comprises a mineral or mineral derivative. In some instances, the nucleic acid may be conjugated to a nanoparticle comprised gold. In some instances the nanoparticle may be a quantum dot. In some instances, the nucleic acid may be conjugated to a nanoparticle comprised of DNA or other nucleic acids. In some instances, the nucleic acid may be attached to a microparticle. In some instances the microparticle may comprise silica. In some instances the microparticle may comprise clay.

In some cases, one or more nucleic acid sequences may be directly conjugated to a ligand or other moiety to enhance uptake, transport, cytoplasmic or nuclear delivery. In some instances, the ligand may be attached to a 5′ end of the nucleic acid duplex. In some instances, the ligand may be attached to a 3′ end of the nucleic acid duplex. In some instances, the ligand maybe attached to both the 5′ end and the 3′ end of the nucleic acid duplex. In some instances, the ligand may be attached to a position within the nucleic acid duplex. In some instances, the ligand may be attached to the non-Watson-Crick face of one of the nucleobases. In some instances, the ligand may be attached to the 5-position of uracil. In some instances, the ligand is attached to multiple positions on the nucleic acid. In some instances, more than one ligand may be attached. In some instances, the multiple copies of the same ligand are attached. In some instances, multiple different ligands are attached.

In some instances, the ligand may be a sugar or polysaccharide. In some instances, the ligand may be GalNAc. In some instances, the ligand may be two GalNAc moieties attached to a single position on the nucleic acid. In some instances, the ligand may be three GalNAc moieties attached to a single position on the nucleic acid. In some instances, the ligand may be more than three GalNAc moieties attached to a single position on the nucleic acid. In some instances, the ligand may be chitin or a derivative thereof. In some instances, the ligand may be mannose or a derivative thereof. In some instances, the ligand may be sialic acid or a derivative thereof.

In some instances, a nucleic acid disclosed herein may be conjugated to a peptide or derivative thereof. In some instances, the peptide may enhance nucleic acid uptake. In some instances, the peptide may enhance endosomal escape. In some instances, the peptide may enhance both uptake and endosomal escape. In some instances, the peptide may enhance delivery to vascular tissue and long-distance transport. In some instances, the peptide may be a cationic. In some instances, the peptide may be a derivative of a viral protein. In some instances, the ligand may be an arginine rich peptide (e.g. TAT). In some instances, the peptide may be histidine rich peptide (e.g.

endoporter). In some instances, the peptide may be a lytic peptide (e.g. melittin). In some instances, the peptide may be a masked peptide that may be activated upon exposure to acidic conditions. In some instances, the peptide may be pH sensitive. In some instances, the peptide may be a derivative of a bacterial protein. In some instances, the peptide may be a derivative of flagellin. In some instances, the peptide may be a derivative of EF-Tu.

In some cases, a nucleic acid disclosed herein may be conjugated to a sterol or sterol derivative. In some instances, the nucleic acid may be conjugated to cholesterol or a cholesterol derivative. In some instances the nucleic acid may be conjugated to a lipid. In some instances the nucleic acid may be conjugated to a single chain lipid. In some instances the nucleic acid may be conjugated to a single chain lipid that contains from 1 to 22 carbons. In some instances the nucleic acid may be conjugated to a single chain lipid that is saturated. In some instances the nucleic acid may be comprised of at least one unsaturated position. In some instances the nucleic acid may be conjugated to a diacyl lipid. In some instances the nucleic acid may be conjugated to diacyl lipid that contains from 1 to 22 carbons. In some instances the nucleic acid may be conjugated to a diacyl lipid that is saturated. In some instances the nucleic acid comprises at least one unsaturated position. In some instances, the nucleic acid may be conjugated to a vitamin or vitamin derivative. In some instances, the nucleic acid may be conjugated to tocopherol.

In some instances, the nucleic acid construct comprises at least about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs. In some instances, the nucleic acid construct comprises at least about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs. In some instances, the nucleic acid construct comprises from 2 to about 100 base pairs. In some instances, the nucleic acid construct comprises from about 10 to about 100 base pairs. In some instances, the nucleic acid construct comprises from about 10 to about 50 base pairs. In some instances, the nucleic acid construct comprises from about 10 to about 40 base pairs. In some instances, the nucleic acid construct comprises from about 10 to about 30 base pairs. In some instances, the nucleic acid construct comprises from about 20 to about 30 base pairs. In some instances, the nucleic acid construct comprises from about (4, 6, 8, or 10) to about 24 base pairs, e.g., about 10 to about 24 base pairs. In some instances, the nucleic acid construct comprises 23 base pairs. In some instances, the nucleic acid construct comprises 24 base pairs.

In some instances, the nucleic acid construct comprises at least about 60%, 65%, 70%, 75%, 80%, 85%, 90%, 91%, 92%, 93%, 94%, 95%, 96%, 97%, 98%, 99%, 99.5%, 99.9%, or 100% sequence identity to a transcription regulatory region of the gene.

In some instances, the transcription regulatory region comprises at least about 10%, 15%, 20%, 25%, 30%, 35%, 40%, 45%, 50%, 55%, 60%, 65%, 70%, 75%, 80%, 85%, or 90% guanine-cytosine content (G-C content). In some instances, the nucleic acid construct comprises at least one nucleotide strand having at least about 10%, 15%, 20%, 25%, 30%, 35%, 40%, 45%, 50%, 55%, 60%, 65%, 70%, 75%, 80%, 85%, or 90% G-C content.

In some instances, the nucleic acid construct comprises at least one polynucleotide strand that may not be phosphorylated at a terminal nucleotide of the polynucleotide strand. In some instances, the nucleic acid construct comprises at least one polynucleotide strand that may not be phosphorylated at a 5′-terminal nucleotide of the polynucleotide strand. In some instances, the nucleic acid construct comprises at least one polynucleotide strand that may not be phosphorylated at a 3′-terminal nucleotide of the polynucleotide strand. In some instances, the nucleic acid construct may be a double stranded nucleic acid construct that comprises two polynucleotide strands, wherein each of the polynucleotide strands may not be phosphorylated at a 5′-terminal nucleotide of the polynucleotide strand. In some instances, the nucleic acid construct may be a double stranded DNA construct that comprises two polynucleotide strands, wherein each of the polynucleotide strands may not be phosphorylated at a 3′-terminal nucleotide of the polynucleotide strand.

In some instances, the gene is at least partially silenced for at least one reproduction cycle. At least partial gene silencing should be understood to mean that the gene is transcribed at a level that is decreased relative to the unmodified gene. In some instances, the unmodified gene is a wild type gene. Measurement of gene transcription may be done by any of methods commonly known in the art, such as measure of mRNA transcript levels. In some instances, the gene is at least partially silenced for at least about: 2, 3, 4, 5, 6, 7, 8, 9, 10, 15, 20, or 30 reproduction cycles. In some instances, the gene may be at least about: 5%, 10%, 15%, 20%, 25%, 30%, 35%, 40%, 45%, 50%, 55%, 60%, 65%, 70%, 75%, 80%, 85%, 90%, 95%, 99%, or 100% silenced, as determined by measurement of a corresponding mRNA transcript level of the gene.

In some instances, disclosed herein is a nucleic acid construct configured to recruit an endogenous epigenetic modifying enzyme or fragment thereof to a portion of said nucleic acid sequence that is targeted for epigenetic modification. “Endogenous” should be understood to mean naturally occurring within the organism that contains the gene. Thus, an “endogenous” epigenetic modifying enzyme should be understood to mean a modifying enzyme that is naturally present in the organism that contains the gene.

In some instances, the nucleic acid construct may comprise a nucleic acid sequence having 50%, 55%, 60%, 65%, 70%, 75%, 80%, 85%, 90%, 95%, 96%, 97%, 98%, 99%, 99.5%, 99.9%, or 100% sequence identity to a nucleic acid sequence of any one SEQ ID NOs: 1-664. In some instances, the nucleic acid construct may comprise a nucleic acid sequence having a nucleic acid sequence of any one SEQ ID NOs: 1-664.

In some cases, an enzyme or fragment thereof may catalyze a transfer of a methyl group to at least one base of a nucleic acid sequence, such as a portion of a gene or a transcription regulatory region of a gene. In some cases, an enzyme or fragment thereof may comprise a methyltransferase. In some cases, an enzyme or fragment thereof may comprise a methyltransferase (MET), a chromomethyltransferase (CMT), a domain rearranged methyltransferase (DRM), any catalytically active fragment thereof, or any combination thereof. In some cases, enzyme may comprise Dnmt3a, Dnmt3b, Dnmt3L, DRM1, DRM2, NtDRM1, Zmet3, Fmu, Dnmt1, MET1, DIM2, DRM2, CMT1, CMT3, any catalytically active fragment thereof, or any combination thereof. A CMT enzyme may comprise OsCMT3, ZCMT3, OsCMT1, NtCMT1, CMT3, any catalytically active fragment thereof or any combination thereof. A MET enzyme may comprise NtMET1, OsMET1-2, ZMET1, OsMET1-1, MET1, any catalytically active fragment thereof, or any combination thereof. A DRM enzyme may comprise OsDRM3, OsDRM2, OsDRM1a, OsDRM1b, ZMET3, NtDRM1, DRM1, DRM2, any catalytically active fragment thereof, or any combination thereof. A DNMT2 enzyme may comprise OsDNMT2, ZMET4, OsCMT2, any catalytically active fragment thereof, or any combination thereof. A nucleic acid sequence may be contacted with any of the forgoing enzymes or fragments thereof thereby yielding an epigenetic modification of at least one base in a nucleic acid sequence (such as a gene of interest). Compositions as described herein, including nucleic acid constructs, may guide endogenous enzymes or fragments thereof to a base of interest in a nucleic acid sequence and thereby direct the contact of the enzyme or fragment thereof with the base of interest such that the enzyme or fragment thereof performs epigenetic modification of the base of interest.

Methods as described herein may comprise methylating one or more bases of a nucleic acid sequence (such as a portion of a gene or a transcription regulatory region of a gene). Methods as described herein may comprise oxidizing one or more bases of a nucleic acid sequence, such as a portion of a gene or a transcription regulatory region of a gene. Methods as described herein may comprise epigenetically modifying at least one base of a nucleic acid sequence, said epigenetically modification being heritable to a plant progeny.

In some cases, an enzyme or fragment thereof may catalyze a change in an epigenetic modification of at least one base of a nucleic acid sequence (such as a portion of a gene or a transcription regulatory region of a gene). A change in an epigenetic modification may include a conversion of a methylated base to a hydroxymethylated base, a carboxylated base, a formylated base, or a combination of any of these. In some cases, an enzyme may comprise a dioxygenase. In some cases, an enzyme may comprise a ten-eleven translocation (TET) family enzyme. In some cases, an enzyme may comprise TET1, TET2, TET3, CXXC finger protein 4 (CXXC4), any catalytically active fragment thereof, or any combination thereof.

An epigenetic modification may occur at any base, such as a cytosine, a thymine, a uracil, an adenine, a guanine, or any combination thereof. The epigenetic modification may be a heritable epigenetic modification, such as an epigenetic modification passed to at least one progeny of an organism. The epigenetic modification may be an engineered epigenetic modification, such as an epigenetic modification that is not non-naturally occurring at a particular base in a nucleic acid sequence of a native organism but one that is introduced into the organism or into an ancestor of an organism using a method as described herein. An organism may comprise a heritable epigenetic modification that may have been previously introduced into a parent organism or an ancestor organism that is retained for at least one reproduction cycle.

In some cases, an epigenetic modification may comprise an oxidation or a reduction. A nucleic acid sequence may comprise one or more epigenetically modified bases. An epigenetically modified base may comprise any base, such as a cytosine, a uracil, a thymine, adenine, or a guanine. An epigenetically modified base may comprise a methylated base, a hydroxymethylated base, a formylated base, or a carboxylic acid containing base or a salt thereof. An epigenetically modified base may comprise a 5-methylated base, such as a 5-methylated cytosine (5-mC). An epigenetically modified base may comprise a 5-hydroxymethylated base, such as a 5-hydroxymethylated cytosine (5-hmC). An epigenetically modified base may comprise a 5-formylated base, such as a 5-formylated cytosine (5-fC). An epigenetically modified base may comprise a 5-carboxylated base or a salt thereof, such as a 5-carboxylated cytosine (5-caC).

A construct may comprise one or more modifications, such as a chemical modification. A construct may comprise about: 1, 2, 3, 4, 5, 6, 7, 8, 9, 10, 11, 12, 13, 14, 15, 16, 17, 18, 19, 20, 25, 30, 35 modifications or more. A construct may comprise from about 1 to about 10 modifications. A construct may comprise from about 1 to about 20 modifications. A construct may comprise from about 5 to about 20 modifications. A modification may be added to a construct to enhance stability of the construct, such as when delivered in vivo. A modification may be added to a construct to enhance update of the construct, such as when delivered in vivo. A portion of bases of a construct may comprise a modification, such as about 10%, 20%, 30%, 40%, 50%, 60%, 70%, 80%, 90% of bases or more. A modification may comprise addition of a methyl group, a fluoro group, a phosphorthioate backbone, or any combination thereof.

A construct may be double stranded. A double stranded constructs may comprise a length of from about 10 basepairs (bp) to about 100 bp. Constructs may comprise a length of from about 10 bp to about 20 bps. Constructs may comprise a length of from about 10 bp to about 30 bps. Constructs may comprise a length of from about 10 bp to about 40 bps. Constructs may comprise a length of from about 10 bp to about 50 bps. Constructs may comprise a length of from about 10 bp to about 60 bps. Constructs may comprise a length of from about 10 bp to about 70 bps. Constructs may comprise a length of from about 10 bp to about 80 bps. Constructs may comprise a length of from about 10 bp to about 90 bps. Constructs may comprise a length of from about 15 bp to about 40 bp. Constructs may comprise a length of from about 15 bp to about 35 bp. Constructs may comprise a length of from about 15 bp to about 60 bp. Constructs may comprise a length of from about 5 bp to about 30 bp. Constructs may comprise a length of from about 5 bp to about 25 bps.

### Detection of Epigenetic Modification

In some aspects, an epigenetic modification disclosed herein such as DNA methylation can be detected. In some instances, the modification, e.g., methylated nucleotide, may be detected by bisulfite sequencing. Bisulfite treatment may convert a cytosine base to uracil and leave methylated cytosines unconverted. Bisulfite treatment may be applied to a portion of a sample and leave a second portion of the sample untreated. Bisulfite treatment may be performed on a sample prior to sequencing or after sequencing. Bisulfite treatment may be utilized alone or in combination with additional techniques to determine a presence, a pattern or a level of epigenetic modification in a nucleic acid sequence, such as a presence, a pattern or a level of methylation in a sequence. Bisulfite sequencing may determine a presence, a pattern, or a level of an epigenetic modification in a nucleic acid sequence. Bisulfite-free sequencing may determine a presence, a pattern, or a level of an epigenetic modification in a nucleic acid sequence. A bisulfite treated sequence may be compared to a comparable sequence having not been treated with bisulfite to determine a presence, a pattern, or a level of epigenetic modification in a nucleic acid sequence.

Sequencing (such as bisulfite-free sequencing) may be utilized alone or in combination with additional techniques to determine a presence, a pattern or a level of epigenetic modification in a nucleic acid sequence, such as a presence, a pattern or a level of methylation in a sequence. Sequencing (such as bisulfite-free sequencing) may determine a presence, a pattern, or a level of an epigenetic modification in a nucleic acid sequence. Sequencing may determine a presence, a pattern, or a level of an epigenetic modification in a nucleic acid sequence. A treated sequence (such as a sequence having a label added to an epigenetic modification) may be compared to a comparable sequence having not been treated to determine a presence, a pattern, or a level of epigenetic modification in a nucleic acid sequence.

The term “sequencing” as used herein, may comprise bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLID sequencing, Ion Torrent semiconductor sequencing, DNA nanoball sequencing, Heliscope single molecule sequencing, single molecule real time (SMRT) sequencing, nanopore DNA sequencing, shot gun sequencing, RNA sequencing, Enigma sequencing, or any combination thereof.

In some cases, a method may comprise sequencing. The sequencing may include bisulfite sequencing or bisulfite-free sequencing.

In some instances, the methods may include storing the sample for a time such as seconds, minutes, hours, days, weeks, months, years or longer after the sample is obtained and before the sample is analyzed. In some cases, the sample obtained from a subject is subdivided prior to the step of storage or further analysis such that different portions of the sample are subject to different downstream methods or processes including but not limited to any combination of methods described herein, storage, bisulfite treatment, amplification, sequencing, labeling, cytological analysis, adequacy tests, nucleic acid extraction, molecular profiling or a combination thereof.

In some cases, a portion of the sample may be stored while another portion of said sample is further manipulated. Such manipulations may include but are not limited to any method as described herein; bisulfite treatment; sequencing; amplification; labeling; molecular profiling; cytological staining; nucleic acid (RNA or DNA) extraction, detection, or quantification; gene expression product (RNA or Protein) extraction, detection, or quantification; fixation; and examination.

In some instances, a methylated nucleotide may be detected by nanopore sequencing. Nanopores may be used to sequence, a sample, a small portion (such as one full gene or a portion of one gene), a substantial portion (such as multiple genes or multiple chromosomes), or the entire genomic sequence of an individual. Nanopore sequencing technology may be commercially available or under development from Sequenom (San Diego, Calif.), Illumina (San Diego, Calif.), Oxford Nanopore Technologies LTD (Kidlington, United Kingdom), and Agilent Laboratories (Santa Clara, Calif.). Nanopore sequencing methods and apparatus are have been described in the art and for example are provided in U.S. Pat. No. 5,795,782, herein incorporated by reference in its entirety.

Nanopore sequencing can use electrophoresis to transport a sample through a pore. A nanopore system may contain an electrolytic solution such that when a constant electric field is applied, an electric current can be observed in the system. The magnitude of the electric current density across a nanopore surface may depend on the nanopore's dimensions and the composition of the sample that is occupying the nanopore. During nanopore sequencing, when a sample approaches and or goes through the nanopore, the samples cause characteristic changes in electric current density across nanopore surfaces, these characteristic changes in the electric current enables identification of the sample. Nanopores used herein may be solid-state nanopores, protein nanopores, or hybrid nanopores comprising protein nanopores or organic nanotubes such as carbon or graphene nanotubes, configured in a solid-state membrane, or like framework. In some instances, nanopore sequencing can be biological, a solid state nanopore or a hybrid biological/solid state nanopore.

In some instances, a biological nanopore can comprise transmembrane proteins that may be embedded in lipid membranes. In some instances, a nanopore described herein may comprise alpha hemolysin. In some instances, a nanopore described herein may comprise mycobacterium smegmatis porin.

Solid state nanopores do not incorporate proteins into their systems. Instead, solid state nanopore technology uses various metal or metal alloy substrates with nanometer sized pores that allow samples to pass through. Solid state nanopores may be fabricated in a variety of materials including but not limited to, silicon nitride (Si₃N₄), silicon dioxide (SiO₂), and the like. In some instances, nanopore sequencing may comprise use of tunneling current, wherein a measurement of electron tunneling through bases as sample (ssDNA) translocates through the nanopore is obtained. In some instances, a nanopore system can have solid state pores with single walled carbon nanotubes across the diameter of the pore. In some instances, nanoelectrodes may be used on a nanopore system described herein. In some instances, fluorescence can be used with nanopores, for example solid state nanopores and fluorescence. For example, in such a system the fluorescence sequencing method converts each base of a sample into a characteristic representation of multiple nucleotides which bind to a fluorescent probe strand-forming dsDNA (were the sample comprises DNA). Where a two-color system is used, each base is identified by two separate fluorescence, and will therefore be converted into two specific sequences. Probes may consist of a fluorophore and quencher at the start and end of each sequence, respectively. Each fluorophore may be extinguished by the quencher at the end of the preceding sequence. When the dsDNA is translocating through a solid state nanopore, the probe strand may be stripped off, and the upstream fluorophore will fluoresce.

In some instances, a 1-100 nm channel or aperture may be formed through a solid substrate, usually a planar substrate, such as a membrane, through which an analyte, such as single stranded DNA, is induced to translocate. In other instances, a 2-50 nm channel or aperture is formed through a substrate; and in still other instances, a 2-30 nm, or a 2-20 nm, or a 3-30 nm, or a 3-20 nm, or a 3-10 nm channel or aperture if formed through a substrate.

In some instances, nanopores used in connection with the methods and devices useful herein are provided in the form of arrays, such as an array of clusters of nanopores, which may be disposed regularly on a planar surface. In some instances, clusters are each in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed.

### Sequence Identity

The terms “homologous,” “homology,” or “percent homology” as used herein refer to the degree of sequence similarity between an amino acid or nucleotide sequence and a reference sequence. As used herein, the term “homology” can be used interchangeably with the term “identity.” In some instances, the degree of sequence similarity herein can be at least about 60%, at least about 65%, at least about 70%, at least about 75%, at least about 80%, at least about 85%, at least about 90%, or at least about 95%, or about 100%. In some instances, percent sequence homology can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application. In some instances, percent homology of sequences can be determined using Smith-Waterman homology search algorithm. Smith & Waterman (1981) Adv. Appl. Math. 2:482-489.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues. Most sequence comparison method over longer sequences are designed to produce optimal alignments that take into consideration possible insertions and deletions without penalizing unduly the overall homology score. This is achieved by inserting “gaps” in the sequence alignment to try to maximize local homology. These more complex methods assign “gap penalties” to each gap that occurs in the alignment so that, for the same number of identical amino acids, a sequence alignment with as few gaps as possible-reflecting higher relatedness between the two compared sequences-will achieve a higher score than one with many gaps. “Affine gap costs” are typically used that charge a relatively high cost for the existence of a gap and a smaller penalty for each subsequent residue in the gap. This is a commonlyused gap scoring system. High gap penalties will of course produce optimized alignments with fewer gaps. Most alignment programs allow the gap penalties to be modified. Typically the default values are used when using such software for sequence comparisons. Calculation of maximum % homology therefore firstly requires the production of an optimal alignment, taking into consideration gap penalties. Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. In some instances, an alignment is determined by the Smith-Waterman homology search algorithm using an affine gap search with a gap open penalty of 12 a gap extension penalty of 2, and a blocks substitution matrix (BLOSUM) of 62.

### Formulations

Also disclosed herein are formulations comprising: one or more nucleic acid constructs, one or more plants or seeds, one or more plant growth regulators, or any salt or solvate thereof, or any combination thereof. The formulation can be as a seed treatment, soil drench, granule formulation, or foliar spray to improve the productivity or alter the phenotype of a wide variety of crops.

Further disclosed herein are formulations comprising one or more nucleic acid construct described herein. The one or more nucleic acid constructs, salts or solvates can at least partially silence a gene a plant or seed. The one or more nucleic acid constructs can alter a phenotype of a gene in a plant or seed.

The formulation comprising one or more nucleic acid constructs, plants, or seeds can further comprise one or more strigolactones, salts, or solvates. The formulation can further comprise one or more plant growth regulators (PGRs), salts or solvates. The formulation can further comprise one or more inhibitors of abscisic acid (ABA) biosynthesis, or any salt or solvate thereof. The formulation can further comprise one or more strigolactones, salts, or solvates and one or more plant growth regulators (PGRs), salts, or solvates. The formulation can further comprise one or more strigolactones, salts, or solvates and one or more inhibitors of abscisic acid (ABA) biosynthesis, or any salt or solvate thereof. The formulation can further comprise one or more plant growth regulators (PGRs), salts, or solvates and one or more inhibitors of abscisic acid (ABA) biosynthesis, or any salt or solvate thereof.

In some cases, the formulations disclosed herein may further comprise one or more additives to facilitate nucleic acid delivery. In some instances, the additive may be a low or high molecular weight polyamine. In some instances, the additive may be polyethylenimine (PEI). In some instances, the additive may be a polyamidoamine (PAMAM) dendrimer. In some instances, the peptide may be a derivative of a viral protein. In some instances, the additive may be a cationic peptide. In some instances, the additive may be an arginine rich peptide (e.g. TAT). In some instances, the additive may be histidine rich peptide (e.g. endoporter). In some instances, the additive may be a lytic peptide (e.g. melittin).

The formulations may comprise at least about 0.1% (w/w) of an nucleic acid construct, plant, or seed, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the nucleic acid construct, plant, or seed.

The formulations may comprise less than about 95% (w/w) of an nucleic acid construct, plant, or seed, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of the nucleic acid construct, plant, or seed.

The formulations may comprise about 0.1%-100% (w/w) of a nucleic acid construct, plant, or seed, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of the nucleic acid construct, plant, or seed.

### Plant Growth Regulators (PGRs)

The formulation can comprise one or more plant growth regulators (PGRs), salts, or solvates. PGRs can be numerous chemical substances that can influence the growth and/or differentiation of plant cells, tissues, or organs. Plant growth regulators can function as chemical messengers for intercellular communication. PGRs can include auxins, gibberellins, cytokinins, abscisic acid (ABA) and ethylene, brassinosteroids, and polyamines. They can work together coordinating the growth and/or development of cells. PGRs can elicit hydraulic enhancement of a plant. PGRs can increase the harvest yield of a plant. Auxins can comprise indole-3-acetic acid (IAA) or its derivative or chemical analog.

The formulation comprising one or more plant growth regulators (PGRs), salts, or solvates can further comprise one or more nucleic acid constructs or modified plants or seeds. The formulation comprising one or more plant growth regulators (PGRs), salts, or solvates can further comprise one or more strigolactones, salts, or solvates. The formulation comprising one or more plant growth regulators (PGRs), salts, or solvates can further comprise one or more inhibitors of abscisic acid (ABA) biosynthesis, or any salt or solvate thereof. The formulation comprising one or more plant growth regulators (PGRs), salts, or solvates can further comprise one or more nucleic acid constructs and one or more strigolactones, salts, or solvates. The formulation comprising one or more plant growth regulators (PGRs), salts, or solvates can further comprise one or more nucleic acid constructs and one or more inhibitors of abscisic acid (ABA) biosynthesis, or any salt or solvate thereof. The formulation comprising one or more plant growth regulators (PGRs), salts, or solvates can further comprise one or more strigolactones, salts, or solvates and one or more inhibitors of abscisic acid (ABA) biosynthesis, or any salt or solvate thereof.

The formulations may comprise at least about 0.1% (w/w) of a plant growth regulator (PGR), salt, or solvate, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the PGR, salt, or solvate.

The formulations may comprise less than about 95% (w/w) of a PGR, salt, or solvate, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of the PGR, salt, or solvate.

The formulations may comprise about 0.1%-100% (w/w) of a PGR, salt, or solvate, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of the PGR, salt, or solvate.

auxins (e.g., IAA)

The formulations may comprise at least about 0.1% (w/w) of an auxin (e.g., IAA), for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the auxin (e.g., IAA).

The formulations may comprise less than about 95% (w/w) of an auxin (e.g., IAA), for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of the auxin (e.g., IAA).

The formulations may comprise about 0.1%-100% (w/w) of an auxin (e.g., IAA), for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of the auxin (e.g., IAA).

gibberellins

The formulations may comprise one or more gibberellins, such as GA1, GA3, GA4, GA7,GA0, ent-gibberellane, ent-kaurene, their derivatives and chemical analogs. The formulations may comprise at least about 0.1% (w/w) of a gibberellin, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the gibberellin.

The formulations may comprise less than about 95% (w/w) of a gibberellin, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of the gibberellin.

The formulations may comprise about 0.1%-100% (w/w) of a gibberellin, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of the gibberellin.

cytokinins

The formulations may comprise one or more cytokinins, such as kinetin, zeatin, 6-benzylaminopurine, diphenylurea, thidiazuron, their derivatives and chemical analogs. The formulations may comprise at least about 0.1% (w/w) of a cytokinin, for example, at least about 0.1%, at least about 0.2%, at least about 0.3%, at least about 0.4%, at least about 0.5%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cytokinin.

The formulations may comprise less than about 95% (w/w) of a cytokinin, for example, less than about 0.1%, less than about 0.2%, less than about 0.3%, less than about 0.4%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of the cytokinin.

The formulations may comprise about 0.1%-100% (w/w) of a cytokinin, for example, about 0.1%-1%, 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, about 90%-95%, about 90%-99%, about 90%-100%, about 95%-99%, or about 99%-100% of the cytokinin.

### Excipients

The formulations disclosed herein may further comprise one or more excipients. The one or more excipients can be one or more pesticides, one or more stabilizers, one or more additives, one or more carriers, one or more dispersants, one or more fertilizer, or any combination thereof. In one example, one or more excipients comprise acetone.

The formulations disclosed herein may further comprise one or more pesticides. The pesticide may be a biopesticide. A biopesticide may be a form of a pesticide that can be based on microorganisms or natural products. A biopesticide may include naturally occurring substances that control pests (biochemical pesticides), microorganisms that control pests (microbial pesticides), and pesticidal substances produced by plants containing added genetic material (plant-incorporated protectants) or PIPs. Examples of biopesticides can include, but are not limited to, gluocosinolate, chitosan, spinosad, alkaloids, terpenoids, phenolics, pyrethroids, rotenoids, nicotinoids, strychnine, scilliroside, canola oil and baking soda. The pesticide may be an organophosphate pesticide, carbamate pesticide, organochlorine insecticide, pyrethroid pesticide, sulfonylurea pesticides, or a combination thereof. The pesticide may be a herbicide, algicide, avidicide, bactericide, fungicide, insecticide, miticide, molluscicide, nematicide, rodenticide, virucide, or a combination thereof.

The formulations may further comprise one or more stabilizers and/or other additives. The stabilizers and/or additives can include, but are not limited to, penetration agents, adhesives, anticaking agents, dyes, dispersants, wetting agents, emulsifying agents, defoamers, antimicrobials, antifreeze, pigments, colorants, buffers, and carriers. The formulations may further comprise surfactants and/or adjuvants.

The formulations may further comprise one or more carriers. Examples of carriers include, but are not limited to, solid carriers, sponges, textiles, and synthetic materials. The synthetic material may be a porous synthetic material. Additional carriers can include organic carriers, such as waxes, linolin, paraffin, dextrose granules, sucrose granules and maltose-dextrose granules. Alternatively, the carrier can be an anorganic carrier such as natural clays, kaolin, pyrophyllite, bentonite, alumina, montmorillonite, kieselguhr, chalk, diatomaceous earths, calcium phosphates, calcium and magnesium carbonates, sulphur, lime, flours or talc. The formulation may be adsorbed into the carrier. The carrier may be characterized by enabling release of the compound, salt, solvate, or formulation.

The formulations may further comprise one or more dispersants. The dispersant may be a negatively charged anion dispersant. The dispersant may be a nonionic dispersant.

The formulations may further comprise fertilizer. The fertilizer may be a chemical fertilizer. The fertilizer may be an organic fertilizer. The fertilizer may be an inorganic fertilizer. The fertilizer may be a granulated or powdered fertilizer. The fertilizer may be a liquid fertilizer. The fertilizer may be a slow-release fertilizer.

The formulations disclosed herein may be formulated as a dry sprayable formulation. Examples of dry sprayable formulations can include, but are not limited to, wettable powders and water dispersible granules. Wettable powders may comprise nucleic acid constructs that have been microionized to powder form. Wettable powders may be applied as suspended particles after dispersion into water. Water dispersible granules may consist of granules that are applied after disintegration or dispersion in water. The water dispersible granules may comprise particles within the range of 0.2 to 4 mm. Water dispersible granules may be formed by agglomeration, spray drying, or extrusion techniques.

The formulations may be formulated as a liquid sprayable formulation. Examples of liquid sprayable formulations can include, but are not limited to, soluble concentrates, suspension concentrates, emulsifiable concentrates, microemulsions, oil dispersions, and microencapsulated particles. Suspension concentrates may comprise a stable suspension of the compound, salt, solvate, or formulation in a fluid usually intended for dilution with water before use. Emulsifiable concentrates may comprise a compound, salt, solvate, or formulation with an emulsifying agent in a water insoluble organic solvate which will form an emulsion when added to water. Microemulsions may comprise a compound, salt, solvate, or formulation with an emulsifying agent in a water insoluble organic solvate which will form a solution/emulsion when added to water.

The formulations may be formulated as a dry spreadable granule formulation. The dry spreadable granule formulation may comprise soil applied granule on inert or fertilizer carriers. The formulations may be formulated as a seed treatment or seed dressing.

The formulations may be formulated for rapid release. The formulations may be formulated for slow release.

### Methods

Also disclosed herein are methods of at least partially silencing a gene in an organism, e.g., plant or seed. The methods can comprise contacting the organism with the nucleic acid constructs disclosed herein, e.g., contacting the organism such as a seed, with a solution of nucleic acid constructs, or directly administering the nucleic acid constructs to the organism such as a leaf of a plant.

The nucleic acid constructs, plants, seeds, and formulations disclosed herein may be used in agriculture. The nucleic acid constructs, plants, seeds, and formulations may be used to promote plant growth. The nucleic acid constructs and formulations disclosed herein may be used for enhancing shoot stability in plants. The nucleic acid constructs, plants, seeds, and formulations may be used for increasing transport capacity in plants. The nucleic acid constructs, plants, seeds, and formulations may be used for increasing drought tolerance of a plant.

Further disclosed herein are methods of improving agriculture comprising applying a formulation comprising a nucleic acid construct to a plant or seed, thereby improving agriculture. Improving agriculture may comprise promoting plant growth. Improving agriculture may comprise enhancing shoot stability in plants. Improving agriculture may comprise increasing transport capacity in plants. Improving agriculture may comprise increasing drought tolerance. Improving agriculture may comprise reducing an application of one or more pesticides. Improving agriculture may comprise terminating application of one or more pesticides. Improving agriculture may comprise reducing watering amounts applied to the plants. Improving agriculture may comprise reducing watering frequency to the plants. Improving agriculture may comprise controlling phytopathogenic fungi. Improving agriculture may comprise controlling unwanted plant growth. Improving agriculture may comprise controlling unwanted insect or mite infestation. Improving agriculture may comprise regulating growth of the plant. Improving agriculture may comprise promoting or stimulating activity in one or more fungi.

Further disclosed herein are methods of controlling phytopathogenic fungi and/or unwanted plant growth and/or unwanted insect or mite infestation and/or for regulating the growth of plants. The methods may comprise use of a formulation comprising a nucleic acid construct disclosed herein to act on the respective pests, their habitat or the plants to be protected from the respective pest, to the soil and/or to unwanted plants and/or the crop plants and/or their habitat.

The nucleic acid constructs described herein may increase plant growth by at least about 5%. The nucleic acid constructs may increase plant growth by at least about 10%. The nucleic acid constructs may increase plant growth by at least about 15%. The nucleic acid constructs may increase plant growth by at least about 20%. The nucleic acid constructs may increase plant growth by at least about 25%. The nucleic acid constructs may increase plant growth by at least about 30%. The nucleic acid constructs may increase plant growth by at least about 50%. The nucleic acid constructs may increase plant growth by at least about 60%, 70%, 80%, 90%, 95%, 100% or more.

The nucleic acid constructs may increase plant growth by at least about 1.5, 2, 3, 4, 5, 6, 7, 8, 9, 10, 11, 12, 13, 14, 15, 16, 17, 18, 19, 20, 25, 30, 40, 50-fold or more. The nucleic acid constructs may increase plant growth by at least about 1.5-fold or more. The nucleic acid constructs may increase plant growth by at least about 2-fold or more. The nucleic acid constructs may increase plant growth by at least about 3-fold or more. The nucleic acid constructs may increase plant growth by at least about 5-fold or more. The nucleic acid constructs may increase plant growth by at least about 10-fold or more. Plant growth may comprise secondary plant growth.

The nucleic acid constructs may enhance shoot growth by at least about 5%. The nucleic acid constructs may enhance shoot growth by at least about 10%. The nucleic acid constructs may enhance shoot growth by at least about 15%. The nucleic acid constructs may enhance shoot growth by at least about 20%. The nucleic acid constructs may enhance shoot growth by at least about 25%. The nucleic acid constructs may enhance shoot growth by at least about 30%. The nucleic acid constructs may enhance shoot growth by at least about 50%. The nucleic acid constructs may enhance shoot growth by at least about 60%, 70%, 80%, 90%, 95%, 100% or more. The nucleic acid constructs may enhance shoot growth by at least about 1.5, 2, 3, 4, 5, 6, 7, 8, 9, 10, 11, 12, 13, 14, 15, 16, 17, 18, 19, 20, 25, 30, 40, 50-fold or more.

The nucleic acid constructs may enhance shoot growth by at least about 1.5-fold or more. The nucleic acid constructs may enhance shoot growth by at least about 2-fold or more. The nucleic acid constructs may enhance shoot growth by at least about 3-fold or more. The nucleic acid constructs may enhance shoot growth by at least about 5-fold or more. The nucleic acid constructs may enhance shoot growth by at least about 10-fold or more.

The nucleic acid constructs may increase transport capacity in plants by at least about 5%. The nucleic acid constructs may increase transport capacity in plants by at least about 10%. The nucleic acid constructs may increase transport capacity in plants by at least about 15%. The nucleic acid constructs may increase transport capacity in plants by at least about 20%. The nucleic acid constructs may increase transport capacity in plants by at least about 25%. The nucleic acid constructs may increase transport capacity in plants by at least about 30%. The nucleic acid constructs may increase transport capacity in plants by at least about 50%. The nucleic acid constructs may increase transport capacity in plants by at least about 60%, 70%, 80%, 90%, 95%, 100% or more.

The nucleic acid constructs may increase transport capacity in plants by at least about 1.5, 2, 3, 4, 5, 6, 7, 8, 9, 10, 11, 12, 13, 14, 15, 16, 17, 18, 19, 20, 25, 30, 40, 50-fold or more. The nucleic acid constructs may increase transport capacity in plants by at least about 1.5-fold or more. The nucleic acid constructs may increase transport capacity in plants by at least about 2-fold or more. The nucleic acid constructs may increase transport capacity in plants by at least about 3-fold or more. The nucleic acid constructs may increase transport capacity in plants by at least about 5-fold or more. The nucleic acid constructs may increase transport capacity in plants by at least about 10-fold or more.

The nucleic acid constructs may increase drought tolerance in plants by at least about 5%. The nucleic acid constructs may increase drought tolerance in plants by at least about 10%. The nucleic acid constructs may increase drought tolerance in plants by at least about 15%. The nucleic acid constructs may increase drought tolerance in plants by at least about 20%. The nucleic acid constructs may increase drought tolerance in plants by at least about 25%. The nucleic acid constructs may increase drought tolerance in plants by at least about 30%. The nucleic acid constructs may increase drought tolerance in plants by at least about 50%. The nucleic acid constructs may increase drought tolerance in plants by at least about 60%, 70%, 80%, 90%, 95%, 100% or more.

The nucleic acid constructs may increase drought tolerance in plants by at least about 1.5, 2, 3, 4, 5, 6, 7, 8, 9, 10, 11, 12, 13, 14, 15, 16, 17, 18, 19, 20, 25, 30, 40, 50-fold or more. The nucleic acid constructs may increase drought tolerance in plants by at least about 1.5-fold or more. The nucleic acid constructs may increase drought tolerance in plants by at least about 2-fold or more. The nucleic acid constructs may increase drought tolerance in plants by at least about 3-fold or more. The nucleic acid constructs may increase drought tolerance in plants by at least about 5-fold or more. The nucleic acid constructs may increase drought tolerance in plants by at least about 10-fold or more.

The nucleic acid constructs may reduce the application of one or more pesticides. Reducing the application of one or more pesticides may comprise reducing an amount of the one or more pesticides that are applied to the plant. The amount of the one or more pesticides applied to the plant may be reduced by at least about 1%, 5%, 10%, 15%, 20%, 25%, 30%, 35%, 40%, 50%, 60%, 70%, 80%, 90%, 95%, or 100%. The amount of the one or more pesticides applied to the plant may be reduced by at least about 10%. The amount of the one or more pesticides applied to the plant may be reduced by at least about 20%. The amount of the one or more pesticides applied to the plant may be reduced by at least about 30%. The amount of the one or more pesticides applied to the plant may be reduced by at least about 50%.

Alternatively, or additionally, reducing the application of the one or more pesticides may comprise reducing a frequency of which the one or more pesticides are applied to the plant. The frequency of which the one or more pesticides are applied to the plant may be reduced by at least about 1%, 5%, 10%, 15%, 20%, 25%, 30%, 35%, 40%, 50%, 60%, 70%, 80%, 90%, 95%, or 100%. The frequency of which the one or more pesticides are applied to the plant may be reduced by at least about 10%. The frequency of which the one or more pesticides are applied to the plant may be reduced by at least about 20%. The frequency of which the one or more pesticides are applied to the plant may be reduced by at least about 30%. The frequency of which the one or more pesticides are applied to the plant may be reduced by at least about 40%. The frequency of which the one or more pesticides are applied to the plant may be reduced by at least about 50%.

Use of the nucleic acid constructs may allow a reduction in the amount of water applied to the plants. The amount of the water applied to the plant may be reduced by at least about 1%, 5%, 10%, 15%, 20%, 25%, 30%, 35%, 40%, 50%, 60%, 70%, 80%, 90%, 95%, or 100%. The amount of the water applied to the plant may be reduced by at least about 10%. The amount of the water applied to the plant may be reduced by at least about 20%. The amount of the water applied to the plant may be reduced by at least about 30%. The amount of the water applied to the plant may be reduced by at least about 50%.

Use of the nucleic acid constructs may allow a reduction in the frequency of which the water is applied to the plant. The frequency of which the water is applied to the plant may be reduced by at least about 1%, 5%, 10%, 15%, 20%, 25%, 30%, 35%, 40%, 50%, 60%, 70%, 80%, 90%, 95%, or 100%. The frequency of which the water is applied to the plant may be reduced by at least about 10%. The frequency of which the water is applied to the plant may be reduced by at least about 20%. The frequency of which the water is applied to the plant may be reduced by at least about 30%. The frequency of which the water is applied to the plant may be reduced by at least about 40%. The frequency of which the water is applied to the plant may be reduced by at least about 50%.

The compound, salt, solvate, formulation disclosed herein may be used to control phytopathogenic fungi. Improving agriculture may comprise controlling unwanted plant growth. Controlling unwanted plant growth may comprise stimulating germination activity of the unwanted plant. The unwanted plant may be a parasitic plant. The unwanted plant may be a root parasitic plant. Examples of parasitic plants can include, but are not limited to, witchweeds (Striga spp.), broomrapes (Orobanche spp, Phelipanche spp), Alectra, dodders, and mistletoes. The unwanted plant may belong to the family Orobanchaceae. The unwanted plant may be witchweed. Examples of unwanted plants can include but are not limited to bindweed, poison sumac, Japanese knotweed, crabgrass, dandelion, plantain plant, ragweed plant, ground ivy, stinging nettle, creeping thistle, poison ivy, bittersweet, tansy, wisteria, ajuga, sweet autumn clematis, barberry, lantana, butterfly bush, common privet, kudzu or English ivy. The unwanted plant may be Orobanche spp. The compound, salt, solvate, or formulation may be applied directly to the unwanted plant. The compound, salt, solvate, or formulation may be applied indirectly to the unwanted plant.

The nucleic acid construct or formulation disclosed herein may be used to control unwanted insect or mite infestation. Examples of insects and mites can include, but are not limited to spiders, gnats, mealybugs, whiteflies, predator mites, spider mites and aphids.

The nucleic acid construct or formulation disclosed herein may be used to regulate growth of the plant. Regulating plant growth may comprise regulating plant breeding. Regulating plant growth may comprise inhibiting shoot branching. Regulating plant growth may comprise regulating one or more plant products. Regulating plant growth may comprise inhibiting root development.

The nucleic acid construct or formulation disclosed herein may be used to promote or stimulate activity in fungi. The compound, salt, solvate, or formulation may stimulate hyphal branching activity of one or more fungi. The compound, salt, solvate, or formulation may induce spore germination of one or more fungi. The one or more fungi may be arbuscular mycorrhizal (AM) fungi.

Further disclosed herein are methods of preserving or extending the life of a plant. Generally, the method may comprise contacting the plant with a nucleic acid construct or formulation disclosed herein.

The nucleic acid construct or formulation may be used to preserve or extend the life of a cut plant. The cut plant may be a flower. The cut plant may be a tree. The cut plant may be bush or shrub. The cut plant may be a vegetable. The compound, salt, solvate, or formulation may be used to preserve or extend the life of an uncut plant. The uncut plant may be a flower. The uncut plant may be a tree. The uncut plant may be bush or shrub. The uncut plant may be a vegetable. The compound, salt, solvate, or formulation may be used to preserve or extend the life of a potted plant. The potted plant may be a flower. The potted plant may be a tree. The potted plant may be bush or shrub. The potted plant may be a vegetable.

The nucleic acid construct or formulation may be used to preserve or extend the life of a flower. Examples of flowers can include, but are not limited to, lilies, daisies, roses, marigolds, Angel's trumpet, phlox, vinca, snapdragons, toadflax, orchids, ferns, black-eyed Susans, blood flowers, blue lobelias, morning glories, poppies, calendulas, geraniums, impatiens, lantanas, larkspurs, calla lilies, hyacinths, azaleas, pointsettias, and begonias.

The nucleic acid construct or formulation may be used to preserve or extend the life of a bush or shrub. Examples of bushes and shrubs can include, but are not limited to, forsynthia, fuchsia, hibiscus, currant, lilac, rose, hydrangea, willow, magnolia, thyme, snowberry, dogwood and holly.

The nucleic acid construct or formulation may be used to preserve or extend the life of a tree. Examples of trees can include, but are not limited to, cypress, poinsettia, palm, fir, pine, spruce, cedar, oak, mulberry, chestnut, hawthorn, poplar, and maple. The tree may be a fir tree. The fir tree may be a Douglas, Balsam or Fraser fir tree. The tree may be a pine tree. The pine tree may be a Scotch or White pine tree. The tree may be a spruce tree. The spruce tree may be a White, Norway or Blue spruce tree. The tree may be a cedar tree. The cedar tree may be a Deodara or Eastern red cedar. The tree may be a cypress tree. The cypress tree may be an Arizona or Leland cypress tree.

The plant may be contacted with a nucleic acid construct or formulation disclosed herein, thereby extending or preserving the life of the plant. Contacting the plant with the nucleic acid construct or formulation may comprise administering the nucleic acid construct or formulation as a spray. Contacting the plant with the nucleic acid construct or formulation may comprise injecting the nucleic acid into the plant. Contacting the plant with the nucleic acid construct or formulation may comprise adding the plant growth material to the irrigation water of the plant. Contacting the plant with the nucleic acid construct or formulation may comprise applying the nucleic acid construct or formulation to the habitat of the plant. Contacting the plant with the nucleic acid construct or formulation may comprise adding the nucleic acid construct or formulation to a plant container (e.g., vase) and placing the plant in the plant container. Contacting the plant with the nucleic acid construct or formulation may comprise adding the nucleic acid construct or formulation to soil.

The life of the plant may be extended by at least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to an untreated plant. The life of the plant may be extended by at least about 20% as compared to an untreated plant. The life of the plant may be extended by at least about 30% as compared to an untreated plant. The life of the plant may be extended by at least about 40% as compared to an untreated plant. The life of the plant may be extended by at least about 50% as compared to an untreated plant. The life of the plant may be extended by at least about 55% as compared to an untreated plant. The life of the plant may be extended by at least about 60% as compared to an untreated plant. The life of the plant may be extended by at least about 65% as compared to an untreated plant. The life of the plant may be extended by at least about 70% as compared to an untreated plant. The life of the plant may be extended by at least about 75% as compared to an untreated plant. The life of the plant may be extended by at least about 80% as compared to an untreated plant. The life of the plant can be determined by measuring the growth time between initial planting of a seed of the plant to the death of the plant.

The life of the plant may be extended by at least about 6, 12, 24, 30, 36, 42, 48, 54, 60, 66, 72, 78, 84, 90, 96, 102, 108, 114, or 120 hours as compared to an untreated plant. The life of the plant may be extended by at least about 24 hours as compared to an untreated plant. The life of the plant may be extended by at least about 36 hours as compared to an untreated plant. The life of the plant may be extended by at least about 48 hours as compared to an untreated plant. The life of the plant may be extended by at least about 72 hours as compared to an untreated plant. The life of the plant may be extended by at least about 96 hours as compared to an untreated plant.

The life of the plant may be extended by at least about 1, 1.5, 2, 2.5, 3, 3.5, 4, 4.5, 5, 5.5, 6, 6.5, or 7 days as compared to an untreated plant. The life of the plant may be extended by at least about 8, 9, 10, 11, 12, 13, 14, 15, 16, 17, 18, 19, or 20 days as compared to an untreated plant. The life of the plant may be extended by at least about 1 day as compared to an untreated plant. The life of the plant may be extended by at least about 2 days as compared to an untreated plant. The life of the plant may be extended by at least about 2.5 days as compared to an untreated plant. The life of the plant may be extended by at least about 3 days as compared to an untreated plant. The life of the plant may be extended by at least about 3.5 days as compared to an untreated plant. The life of the plant may be extended by at least about 4 days as compared to an untreated plant. The life of the plant may be extended by at least about 4.5 days as compared to an untreated plant.

The life of the plant may be extended by at least about 1, 1.5, 2, 2.5, 3, 3.5, 4, 4.5, 5, 5.5, 6, 6.5, or 7 weeks as compared to an untreated plant. The life of the plant may be extended by at least about 8, 9, 10, 11, 12, 13, 14, 15, 16, 17, 18, 19, or 20 weeks as compared to an untreated plant. The life of the plant may be extended by at least about 1, 1.5, 2, 2.5, 3, 3.5, 4, 4.5, 5, 5.5, 6, 6.5, or 7 months as compared to an untreated plant. The life of the plant may be extended by at least about 8, 9, 10, 11, 12, 13, 14, 15, 16, 17, 18, 19, or 20 months as compared to an untreated plant.

Preserving or extending the life of the plant may comprise reducing wilting of the plant. Reducing wilting of the plant may comprise reducing flower or leaf rolling of the plant. The wilting of the plant may be reduced by at least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to an untreated plant. The wilting of the plant may be reduced by at least about 10% as compared to an untreated plant. The wilting of the plant may be reduced by at least about 30% as compared to an untreated plant. The wilting of the plant may be reduced by at least about 50% as compared to an untreated plant. The wilting of the plant may be reduced by at least about 70% as compared to an untreated plant. The wilting of the plant may be reduced by at least about 80% as compared to an untreated plant.

A sign of plant stress may include wilting of the plant. For example, stressed plants may have rolled leaves or petals. The plant growth materials disclosed herein may promote the life of the plant by reducing the wilting of the plant. Reducing the wilting of the plant may comprise delaying the wilting of the plant as compared to an untreated plant. For example, an untreated cut plant may show signs of wilting within 36 hours of being cut, however, a cut plant treated with a plant growth material may have delayed wilting. The wilting of the plant may be delayed by at least about 2, 3, 4, 5, 6, 7, 8, 9, 10, 11, 12, 13, 14, 15, 16, 17, 18, 19, 20, 21, 22, 23, or 24 hours as compared to an untreated plant. The wilting of the plant may be delayed by at least about 12 hours as compared to an untreated plant. The wilting of the plant may be delayed by at least about 24 hours as compared to an untreated plant. The wilting of the plant may be delayed by at least about 36 hours as compared to an untreated plant. The wilting of the plant may be delayed by at least about 48 hours as compared to an untreated plant.

An additional sign of plant stress may include reduced turgidity. Turgidity may refer to pressure caused by the osmotic flow of water from an area of low solute concentration outside of the cell into the cell cell's vacuole. Turgidity may be used by plants to maintain rigidity. Often, healthy plants are turgid, whereas, unhealthy plants are less turgid. Preserving or extending the life of the plant may comprise prolonging or maintaining the turgidity of the plant. The turgidity of the plant may be greater than the turgidity of an untreated plant. The turgidity of the plant may be at least about 1%, 5%, 10%, 15%, 20%, 25%, 30%, 35%, 40%, 45%, 50%, 55%, 60%, 65%, 70%, 75%, 80%, 85%, 90%, 95%, or 97% greater than the turgidity of an untreated plant. The turgidity of the plant may be at least about 10% greater than the turgidity of an untreated plant. The turgidity of the plant may be at least about 15% greater than the turgidity of an untreated plant. The turgidity of the plant may be at least about 25% greater than the turgidity of an untreated plant. The turgidity of the plant may be at least about 35% greater than the turgidity of an untreated plant. The turgidity of the plant may be at least about 45% greater than the turgidity of an untreated plant. The turgidity of the plant may be at least about 60% greater than the turgidity of an untreated plant. The turgidity of the plant may be at least about 75% greater than the turgidity of an untreated plant.

A stressed plant may also show a reduction in the turgid state. The turgid state may refer to a period of time in which the plant maintains its rigidity. The rigidity of the plant may refer to the rigidity of the stem of the plant. For example, as cut plants die, the stem of the plant may be less rigid, thereby causing the cut plant to fall over or bend. A stressed plant may be unable to hold itself upright. Preserving or extending the life of the plant may comprise prolonging the turgid state of the plant. The turgid state of the plant may be increased by at least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to an untreated plant. The turgid state of the plant may be increased by at least about 20% as compared to an untreated plant. The turgid state of the plant may be increased by at least about 30% as compared to an untreated plant. The turgid state of the plant may be increased by at least about 40% as compared to an untreated plant. The turgid state of the plant may be increased by at least about 50% as compared to an untreated plant.

The turgid state of the plant may be increased by at least about 2, 3, 4, 5, 6, 7, 8, 9, 10, 11, 12, 13, 14, 15, 16, 17, 18, 19, 20, 21, 22, 23, or 24 hours as compared to an untreated plant. The turgid state of the plant may be increased by at least about 6 hours as compared to an untreated plant. The turgid state of the plant may be increased by at least about 12 hours as compared to an untreated plant. The turgid state of the plant may be increased by at least about 24 hours as compared to an untreated plant.

A stressed plant may lose leaves or petals. Contacting a plant with a plant growth material may reduce or delay the loss of one or more petals or leaves of the plant. For example, an untreated plant may lose 50% of its leaves or petals, whereas a treated plant may lose 10-25% of its leaves or petals. The loss of the one or more petals of the plant may be reduced by least about 1%, 5%, 10%, 15%, 20%, 25%, 30%, 35%, 40%, 45%, 50%, 55%, 60%, 65%, 70%, 75%, 80%, 85%, 90%, 95%, or 97% as compared to the loss of the one or more petals of an untreated plant. The loss of the one or more petals of the plant may be reduced by least about 10% as compared to the loss of the one or more petals of an untreated plant. The loss of the one or more petals of the plant may be reduced by least about 20% as compared to the loss of the one or more petals of an untreated plant. The loss of the one or more petals of the plant may be reduced by least about 35% as compared to the loss of the one or more petals of an untreated plant. The loss of the one or more petals of the plant may be reduced by least about 50% as compared to the loss of the one or more petals of an untreated plant. The loss of the one or more petals of the plant may be reduced by least about 60% as compared to the loss of the one or more petals of an untreated plant. The loss of the one or more petals of the plant may be reduced by least about 70% as compared to the loss of the one or more petals of an untreated plant.

The loss of the one or more petals of the plant may be delayed by at least about 2, 3, 4, 5, 6, 7, 8, 9, 10, 11, 12, 13, 14, 15, 16, 17, 18, 19, 20, 21, 22, 23, or 24 hours as compared to the loss of one or more petals of an untreated plant. The loss of the one or more petals of the plant may be delayed by at least about 25, 30, 35, 40, 45, 50, 55, 60, 65, 70, 75, 80, 85, 90, 95 or 100 hours as compared to the loss of one or more petals of an untreated plant. The loss of the one or more petals of the plant may be delayed by at least about 6 hours as compared to the loss of one or more petals of an untreated plant. The loss of the one or more petals of the plant may be delayed by at least about 12 hours as compared to the loss of one or more petals of an untreated plant. The loss of the one or more petals of the plant may be delayed by at least about 18 hours as compared to the loss of one or more petals of an untreated plant. The loss of the one or more petals of the plant may be delayed by at least about 36 hours as compared to the loss of one or more petals of an untreated plant. The loss of the one or more petals of the plant may be delayed by at least about 48 hours as compared to the loss of one or more petals of an untreated plant. The loss of the one or more petals of the plant may be delayed by at least about 60 hours as compared to the loss of one or more petals of an untreated plant. The loss of the one or more petals of the plant may be delayed by at least about 72 hours as compared to the loss of one or more petals of an untreated plant. The loss of the one or more petals of the plant may be delayed by at least about 96 hours as compared to the loss of one or more petals of an untreated plant.

A stressed plant may show signs of discoloration. The stressed plant may appear brownish. Alternatively, or additionally, the stressed plant shows a reduction in the appearance of green leaves. The chlorophyll content of the stressed plant may also be reduced. Preserving or extending the life of the plant may comprise maintaining the chlorophyll content of the plant. For example, a reduction in the chlorophyll content of an untreated plant may appear within 48 hours of being cut. However, a reduction in the chlorophyll content of a treated plant may appear after 60 hours of being cut. The chlorophyll content of the plant may be maintained for at least about 2, 3, 4, 5, 6, 7, 8, 9, 10, 11, 12, 13, 14, 15, 16, 17, 18, 19, 20, 21, 22, 23, or 24 hours. The chlorophyll content of the plant may be maintained for at least about 6 hours. The chlorophyll content of the plant may be maintained for at least about 12 hours. The chlorophyll content of the plant may be maintained for at least about 24 hours. Discoloration such as leaf firing (premature yellowing) may occur as a result of poor nutrient availability, and can be an indicator of poor plant health. For, example, leaf firing may be a result of nitrogen deficiency.

Preserving or extending the life of the plant may comprise reducing or delaying the loss of the chlorophyll content of the plant. The chlorophyll content of the plant may be greater than the chlorophyll content of an untreated plant. The chlorophyll content of the plant may be at least about 1%, 5%, 10%, 15%, 20%, 25%, 30%, 35%, 40%, 45%, or 50% greater than the content of an untreated plant. The chlorophyll content of the plant may be at least about 55%, 60%, 65%, 70%, 75%, 80%, 85%, 90%, 95% or 97% greater than the content of an untreated plant. The chlorophyll content of the plant may be at least about 20% greater than the content of an untreated plant. The chlorophyll content of the plant may be at least about 30% greater than the content of an untreated plant. The chlorophyll content of the plant may be at least about 40% greater than the content of an untreated plant. The chlorophyll content of the plant may be at least about 50% greater than the content of an untreated plant. The chlorophyll content of the plant may be at least about 60% greater than the content of an untreated plant. The chlorophyll content of the plant may be at least about 1.5, 2, 2.5, 3, 3.5, 4, 4.5, 5, 5.5, 6, 6.5, 7, 8, 9, or 10-fold greater than the content of an untreated plant.

The chlorophyll content of the plant may be at least about 11, 12, 13, 14, 15, 16, 17, 18, 19, 20, 25, 30, 35, 40, 45, 50, 55, 60, 65, 70, 75, 80, 85, 90, 95 or 100-fold greater than the content of an untreated plant. The chlorophyll content of the plant may be at least about 2-fold greater than the content of an untreated plant. The chlorophyll content of the plant may be at least about 3-fold greater than the content of an untreated plant. The chlorophyll content of the plant may be at least about 4-fold greater than the content of an untreated plant. The chlorophyll content of the plant may be at least about 5-fold greater than the content of an untreated plant. The chlorophyll content of the plant may be at least about 10-fold greater than the content of an untreated plant.

The loss of the chlorophyll content of the plant may be delayed by at least about 2, 3, 4, 5, 6, 7, 8, 9, 10, 11, 12, 13, 14, 15, 16, 17, 18, 19, 20, 21, 22, 23, or 24 hours as compared to the loss of the chlorophyll content of an untreated plant. The loss of the chlorophyll content of the plant may be delayed by at least about 30, 35, 40, 45, 50, 55, 60, 65, 70, 75, 80, 85, 90, 95, or 100 hours as compared to the loss of the chlorophyll content of an untreated plant. The loss of the chlorophyll content of the plant may be delayed by at least about 6 hours as compared to the loss of the chlorophyll content of an untreated plant. The loss of the chlorophyll content of the plant may be delayed by at least about 12 hours as compared to the loss of the chlorophyll content of an untreated plant. The loss of the chlorophyll content of the plant may be delayed by at least about 24 hours as compared to the loss of the chlorophyll content of an untreated plant. The loss of the chlorophyll content of the plant may be delayed by at least about 36 hours as compared to the loss of the chlorophyll content of an untreated plant. The loss of the chlorophyll content of the plant may be delayed by at least about 48 hours as compared to the loss of the chlorophyll content of an untreated plant. The loss of the chlorophyll content of the plant may be delayed by at least about 60 hours as compared to the loss of the chlorophyll content of an untreated plant. The loss of the chlorophyll content of the plant may be delayed by at least about 72 hours as compared to the loss of the chlorophyll content of an untreated plant.

The loss of the chlorophyll content of the plant may be less than the loss of the chlorophyll content of an untreated plant. The loss of the chlorophyll content of the plant may be at least about 1%, 5%, 10%, 15%, 20%, 25%, 30%, 35%, 40%, 45%, 50%, 55%, or 60% less than the loss of the chlorophyll content of an untreated plant. The loss of the chlorophyll content of the plant may be at least about 65%, 70%, 72%, 75%, 77%, 80%, 85%, 90%, 92%, 95%, or 97% less than the loss of the chlorophyll content of an untreated plant. The loss of the chlorophyll content of the plant may be at least about 5% less than the loss of the chlorophyll content of an untreated plant. The loss of the chlorophyll content of the plant may be at least about 10% less than the loss of the chlorophyll content of an untreated plant. The loss of the chlorophyll content of the plant may be at least about 20% less than the loss of the chlorophyll content of an untreated plant. The loss of the chlorophyll content of the plant may be at least about 30% less than the loss of the chlorophyll content of an untreated plant. The loss of the chlorophyll content of the plant may be at least about 40% less than the loss of the chlorophyll content of an untreated plant. The loss of the chlorophyll content of the plant may be at least about 50% less than the loss of the chlorophyll content of an untreated plant. The loss of the chlorophyll content of the plant may be at least about 60% less than the loss of the chlorophyll content of an untreated plant.

The loss of the chlorophyll content of the plant may be at least about 1.5, 2, 2.5, 3, 3.5, 4, 4.5, 5, 5.5, 6, 6.5, 7, 7.5, 8, 8.5, 9, 9.5, or 10-fold less than the loss of the chlorophyll content of an untreated plant. The loss of the chlorophyll content of the plant may be at least about 11, 12, 13, 14, 15, 16, 17, 18, 19, 20, 25, 30, 35, 40, 45, 50, 55, 60, 65, 70, 75, 80, 85, 90, 95 or 100-fold less than the loss of the chlorophyll content of an untreated plant. The loss of the chlorophyll content of the plant may be at least about 2-fold less than the loss of the chlorophyll content of an untreated plant. The loss of the chlorophyll content of the plant may be at least about 3-fold less than the loss of the chlorophyll content of an untreated plant. The loss of the chlorophyll content of the plant may be at least about 5-fold less than the loss of the chlorophyll content of an untreated plant. The loss of the chlorophyll content of the plant may be at least about 10-fold less than the loss of the chlorophyll content of an untreated plant.

The nucleic acid construct or formulation may be applied directly to the plant. The nucleic acid construct or formulation may be applied to one or more parts of the plant. The one or more parts of the plant may comprise a terminal bud, flower, lateral bud, leaf blade, leaf axil, node, internode, petiole, primary root, lateral root, root hair, root cap, or a combination thereof. The formulations may be applied to the leaf blade of the plant. The formulations may be applied to the root of the plant.

Alternatively, or additionally, the nucleic acid construct or formulation can be applied to a soil. The formulation may be applied to an area around the plant. The area around the plant may comprise soil. The area around the plant may comprise an adjacent plant. The formulation may be applied to a soil before placing a plant or seed in the soil. The formulation may be applied to bacterial consortium present in the soil. The formulation may be applied with additional bacteria to supplement the natural bacterial consortium in the soil.

The nucleic acid construct or formulation may be applied to a plant that is susceptible to a parasitic weed. Examples of plants include, but are not limited to, corn, rice, sorghum, millets, and sugar cane. The plant may be corn. The plant may be tobacco. The plant may be rice.

The nucleic acid construct or formulation may improve taste or texture of an edible product of the plant. In non-limiting examples, the targeted gene may be control sugar and/or starch biosynthesis or storage. The targeted gene may control tannin biosynthesis. The targeted gene may control anthocyanin biosynthesis. The target gene may control metabolite biosynthesis.

The nucleic acid construct may improve nutritional content of the plant, for example, by increasing or decreasing sugar, starch, protein, and/or fat content of an edible product of the plant, enhancing the accumulation of vitamins and/or minerals in the plant. The targeted gene may control sugar biosynthesis, carbohydrate biosynthesis and/or storage, protein biosynthesis and/or degradation, and/or secondary metabolite biosynthesis.

The nucleic acid construct may increase the shelf life of an edible product of the plant, for example by decreasing ethylene biosynthesis in the plant. The targeted gene may control ethylene biosynthesis.

The nucleic acid construct or formulation may be applied as a seed coating. The nucleic acid construct or formulation may be applied as a seed treatment. The nucleic acid construct or formulation may be applied as a seed dressing. The nucleic acid construct or formulation may be applied as a spray. The nucleic acid construct or formulation may be applied as a foliar spray. The nucleic acid construct or formulation may be applied as a powder. The powder may be a wettable powder.

In some instances, the measurements described herein can be made at a temperature of about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 31, 32, 33, 34, 35, 36, 37, 38, 39, or 40° C.

### Kits

Also provided are kits that find use in practicing the subject methods, as mentioned above. A kit can include one or more of the compositions described herein. A kit can include at least nucleic acid construct. A kit can include at least one engineered plant or seed.

A kit can include one or more reagents for performing administration of nucleic acid constructs to plants or seeds (e.g., polynucleotides, buffers, cations, etc.), and the like. Additional reagents that are desired in the protocol to be practiced with the kit components may be present. Such additional reagents include, but are not limited to, one or more of the following an enzyme or combination of enzymes such as a polymerase, reverse transcriptase, nickase, restriction endonuclease, uracil-DNA glycosylase enzyme, enzyme that methylates or demethylates DNA, endonuclease, ligase, etc.

The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, such as printed information on a suitable medium or substrate (e.g., a piece or pieces of paper on which the information is printed), in the packaging of the kit, in a package insert, etc. Yet another means would be a computer readable medium (e.g., diskette, CD, etc.), on which the information has been recorded. Yet another means that may be present is a website address which may be used via the internet to access the information at a removed site.

### Communicating Results

The present disclosure provides the communication of assay results or diagnoses or both to technicians, physicians or subjects, for example. In certain instances, computers will be used to communicate results of the methods herein to interested parties, e.g., customers, technicians, physicians and their subjects, etc. In some instances, the methods can be performed, or results analyzed in a country or jurisdiction which differs from the country or jurisdiction to which the results are communicated. In some instances, a result may be communicated to the subject as soon as possible after the diagnosis is obtained. The results may be sent to a subject by email or communicated to the subject by phone. A computer may be used to communicate the result by email or phone. In certain instances, the message containing result may be generated and delivered automatically to the subject using a combination of computer hardware and software which will be familiar to artisans skilled in telecommunications. In certain instances or some of the method steps, including the preparation of plants and seeds, and communicating of assay results, may be carried out in diverse (e.g., foreign) jurisdictions.

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular instances only, and is not intended to limit the scope of the methods and compositions described herein, which will be limited only by the appended claims. While preferred instances of the present disclosure have been shown and described herein, it will be obvious to those skilled in the art that such instances are provided by way of example only. Numerous variations, changes, and substitutions will now occur to those skilled in the art without departing from the disclosure. It should be understood that various alternatives to the instances of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any instance can be combined with any other instance. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### EXAMPLES

### Example 1—Preparation of CATS Oligonucleotide Nucleic Acid Construct Solutions

Oligonucleotides were custom synthesized. 96 well oligo plates were obtained; one plate for each locu of interest. Pooled stocks for each oligo plate were made by taking 20 uL of a 400 uM stock from each well of that plate and combining these in a 2 mL tube. Afterwards, each pooled sock was combined in a 12 mL sterile falcon tube, mixed by pipetting, and briefly centrifuged to collect all contents. This pooled oligo mix was then pipetted into two eight-well PCT strip tubes and spun down. Tubes were placed in a PTC-200 Thermal cycler at 95 degrees for 1 minute to denature contents, and then cooled at room temperature for 10 minutes. Contents of strip tubles were again combined in a fresh sterile 12 mL falcon tube, mixed by pipetting and centrifuged briefly to collect contents. Applications of serial dilution of this full-strength oligo mix were applied to plants.

Representative sequences of oligonucleotides are shown in SEQ ID NOs 1-664.

### Example 2—Application of CATS Nucleotides to Plants and Seeds

### Vacuum Treatment Method in Corn

Five 20 mL clear glass scintillation vials were obtained, and six B73 Maize seeds were place in each vial, lying flat. Seeds in vials 1-4 received 900 uL of an oligo treatment (full strength, 1/10, 1/100, and 1/1000, respectively) and seeds in vial 5 received 900 uL of water. After incubating for 20 minutes, caps were removed from all five vials, which were then placed in an airtight chamber and vacuumed 18 hours at 400 mbar. After 18 hours, vials were removed from the vacuum and each seed was planted in 100 mm2 plastic pot filled with moistened Sunshine Mix No. 4. Seeds were left to germinate under next light Veg 8 LED panels set to long day conditions.

### Germination and Outgrowth in Solution Method

Five 20 mL clear glass scintillation vials were obtained, and six B73 Maize seeds were placed in each vial, lying flat. Seeds in vials 1-4 received 900 uL of an oligo treatment (full strength, 1/10, 1/100, and 1/1000, respectively) and seeds in vial 5 received 900 uL of water. Vials were placed in the dark with caps on and seeds were left to germinate, for 4 days. Once epicotyls had emerged, vials were removed from dark, embryos were removed from treatment solutions, and each was planted in 100 mm2 plastic pot filled with moistened Sunshine Mix No. 4.

### Syringe Infiltration Method

CATS oligonucleotide solutions described above can be administered directly to plant tissue by syringe in order to locally deliver the oligonucleotides to a particular location on the plant.

### Example 3—Targeting the Phytoene Desaturase (PDS1) in Maize

CATS oligonucleotides were constructed in to target the phytoene desaturase, an enzyme involved in the synthesis of carotenoids in plants. Mutations or knockouts of this gene result in albino phenotypes.

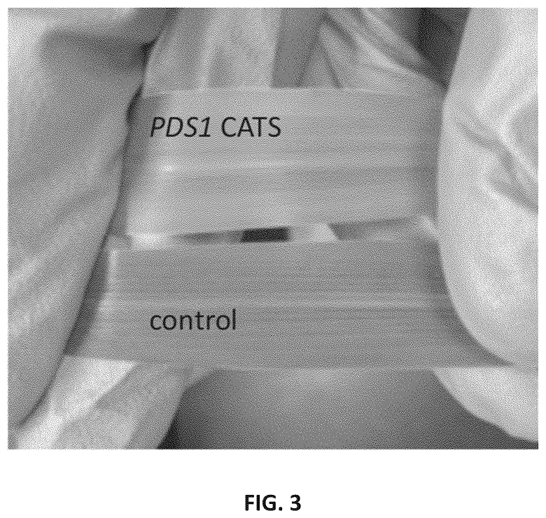

A total of 62 oligonucleotides (31 oligonucleotide pairs, shown in SEQ ID NOs: 1-62) were synthesized targeting transcription regulatory regions upstream of PDS1 gene open reading frame in a maize plant. The mixture of oligonucleotides was applied to maize seeds, and seedlings grown from the seeds. PDS1-CATS plants showed pale green leaf phenotype compared to the uncontacted control plants (FIG. 3).

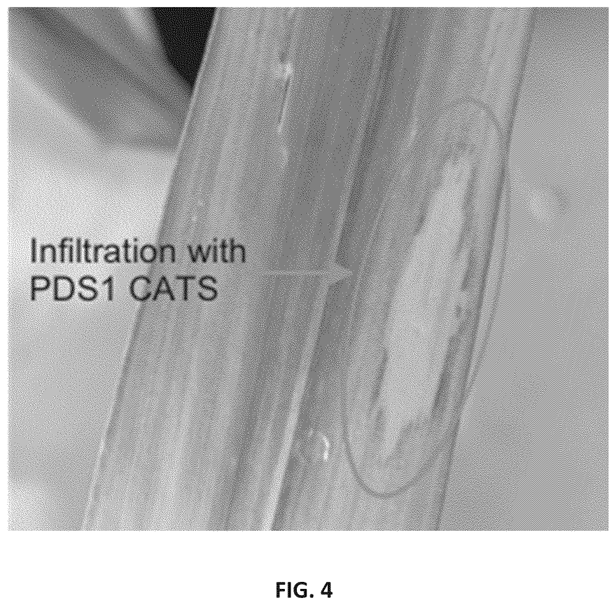

Administration of CATS oligonucleotides directly to a maize leaf by syringe infiltration results in local loss of pigmentation (FIG. 4).

### Confirmation of DNA Methylation by Bisulfite Sequencing

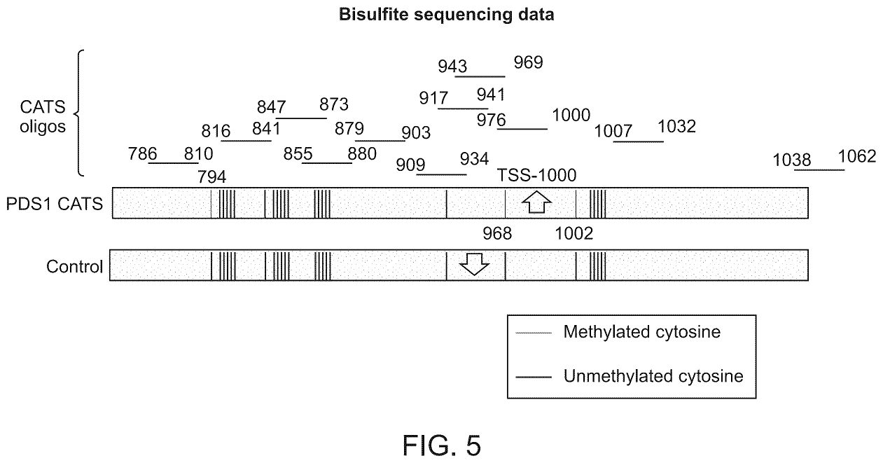

Leaf tissue from sterile scissors were cut and placed in a chilled (LN2) closed cap tube with one medium glass bead. Leaf tissue was bead beated for 1:30 at 1800 RPN. DNA was extracted using the PureLink Plant Total DNA extraction kit. A total of 20 μL of DNA from the extraction was used with Zymo Research Corp Easy DNA Methylation kit. Human standards were used to confirm the kit was working. Primers were designed using MethPrimer2 (http://www.urogene.org/cgi-bin/methprimer2/MethPrimer.cgi) and allowing degeneracy in the primers if necessary. Bi-sulfite treated DNA from the kit was used to perform a PCR reaction using 12.5 μL Zymotaq, 1 μL primer-F, 1 μL primer-R, 4 μL BS treated DNA and 7.5 μL water. The first set of BS-PCR primers were amplified at 50° C. and cover GC islands. Amplicons were sequence cloned to confirm. Initial analysis of the sequenced data indicates that some of the cytosines were methylated (FIG. 5).

### Example 4-Targeting the LZY1 Gravitropism Regulator in Maize

The plant regulator Lazyl (LZY1) is known to control the plant's shoot gravitropism. In wildtype plants, shoots grow in the opposite direction of gravity and respond to changes in orientation by bending to maintain this directionality. In contrast, mutants in LZY1 expression do not respond to changes in orientation. CATS oligos were designed to target the LZY1 regulator. A total of 132 oligonucleotides (67 oligonucleotide pairs, shown in SEQ ID NOs: 63-194) were synthesized targeting transcription regulatory regions upstream of LZY1 gene open reading frame in a maize plant. The mixture of oligonucleotides was applied to maize seeds, and seedlings grown from the seeds.

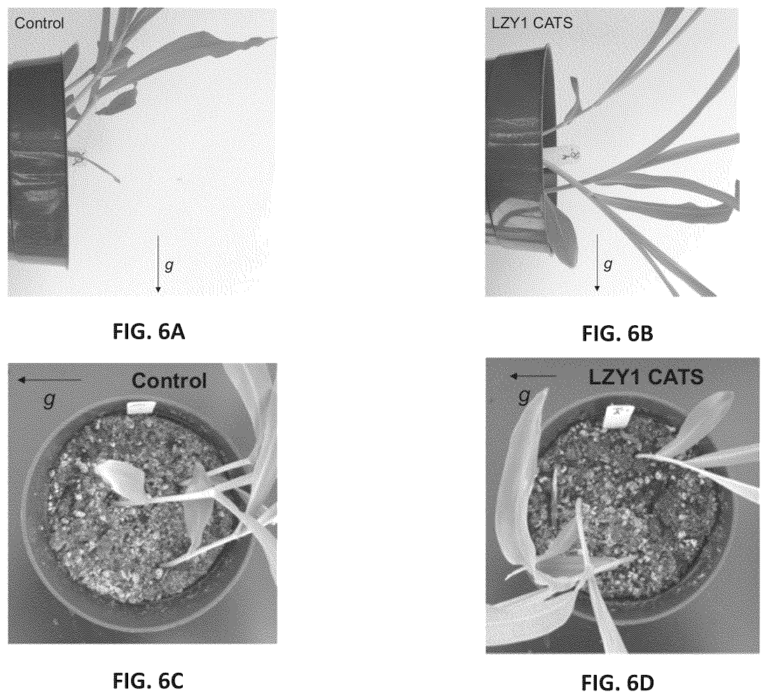

As shown in FIG. 6B and 6D, CATS: LZY1 seedlings display reduced or absent gravitropism when turned on their side, as compared to unmodified plants (FIG. 6A and 6C), where the control plants display a strong gravitropic response.

### Example 5—Targeting Polyphenol Oxidases in Potato

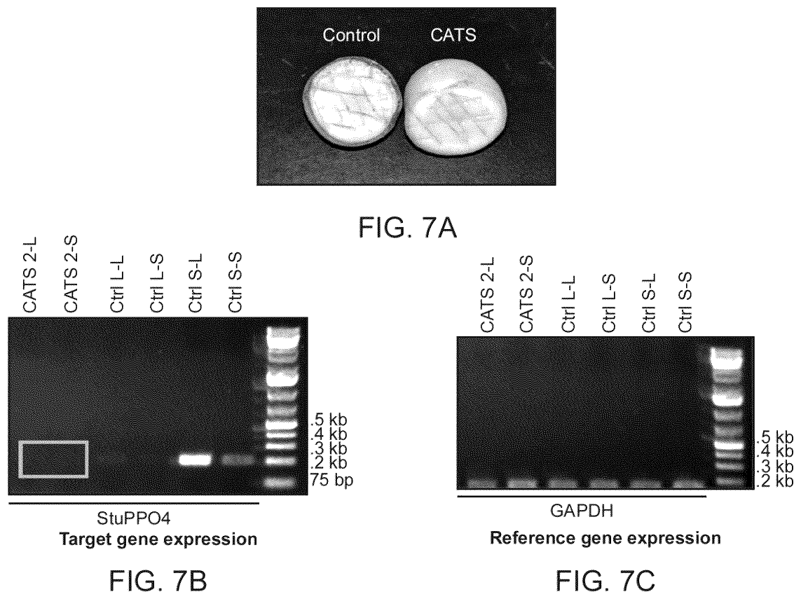

The polyphenol enzyme oxidase (PPO) enzymes in plants are responsible for the enzymatic browning phenotype that occurs after cutting or wounding of the plant. Non-browning crops are desirable from a consumer acceptance and food waste perspective. To achieve non-browning potatoes, CATS oligos were designed to simultaneously target the 7 PPO alleles in potato (Solanum tuberosum). A total of 210 individual oligos (105 oligo pairs, shown in SEQ ID NOs: 195-404) were applied to seed potatoes using the ‘outgrowth in solution’ method above. Plants were grown in greenhouse conditions and tubers were harvested approximately 3 months after planting. RT-PCT analysis of PPO mRNA levels indicated that CATS plants had reduced levels of the transcript (FIG. 7B), and phenotype analysis shows that tubers from CATS plants had greatly reduced enzymatic browning when cut, as compared to the potatoes of unmodified control plants (FIG. 7A).

### Example 6—Creation of Short Stature Corn

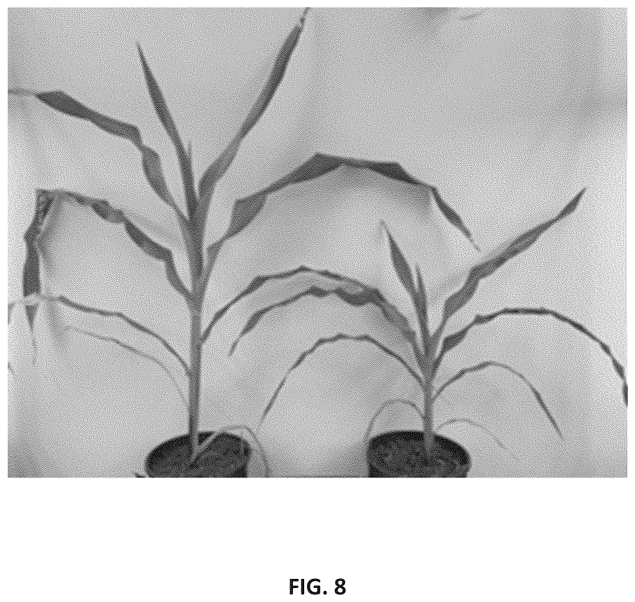

CATS oligos were designed to target genes involved in the biosynthesis of brassinosteroids and involved in the gibberellin-brassinosteroid balance (BWF1 and BR2). A total of 180 individual oligos (90 oligo pairs, shown in SEQ ID NOs: 405-584) were applied to maize seeds. Plants grown from contacted seeds were shorter than control plants not contacted with the CATS oligos (FIG. 8).

### Example 7—Targeting the Old Gold Gene in a Population of Tomato Plants

### Oligo Treatment and Plant Growth Conditions

Oligos were designed to target the microTom genome (˜500 bp upstream and downstream of the Old Gold translation start site, shown in SEQ ID NOs: 585-684) with non-overlapping oligos across this ˜1 kb region. Complementary oligos were annealed by incubating at 95° C. for 10 mins with gradual cooling to room temperature, then annealed oligo pairs were pooled for each treatment. Seeds were incubated with 900 ul of oligos at 250 μM concentration (0.5x TE buffer) either targeting Old Gold (treated) or with random oligos as a control (untreated). Seeds were incubated at room temperature in the dark for 3 days before planting in Sunshine Mix soil #4 and grown under 16 hr photoperiods until two true leaves had established (˜2 weeks). 50-100 mg of true leaf tissue was harvested from each sample that emerged and frozen in liquid nitrogen before being stored at-80 for later analysis.

### RNA Isolation

Total RNA isolation from the starting material was collected using the PureLink Pro 96 total RNA Purification Kit (ThermoFisher Scientific) according to the manufacturer's instructions. Total RNA was eluted in RNase free water. Samples with tissue that were unable to be isolated were discarded. Total RNA was quantified using the Quant-iT™ RNA Assay Kit, broad range,

(ThermoFisher Scientific) using fluorescence on the SpectraMax iD3 plate reader. Quantified total RNA was diluted to Ing using RNase free water. RNA lower than Ing/uL were discarded. After isolation and quantification were complete, there were population sizes of 40 samples of 1 KB treatment; 45 samples of 2.5 KB; 44 samples of RO (random oligonucleotides) control; and 11 samples of Water control.

### Quantitative Real-time PCR Conditions

RT-qPCR was carried out in a 96-well optical plate using Quantstudios 6 Flex Real-Time PCR Systems (Applied Biosystems). Reactions were performed using Luna Universal One-Step RT-qPCR Kit, which uses Luna WarmStart Reverse Transcriptase and SYBR Green I (New England Biolabs); 10 μM of each primer; RNase free water; and 1 μL of diluted RNA (1 ng/uL) in a final volume of 20 μL. The following thermal cycling conditions were used for all amplifications (following the Luna Universal One-Step RT-qPCR Kit manual): 55° C. for 10 minutes for reverse transcription, 95° C. for 1 minute for denaturation, 40 amplification cycles of 95° C. for 10 seconds and 60° C. for 1 minute, and a melt curve of various stages of 60-95° C. All samples were prepared in technical triplicates and three non template controls of RNase free water for each qPCR plate.

### Data Analysis

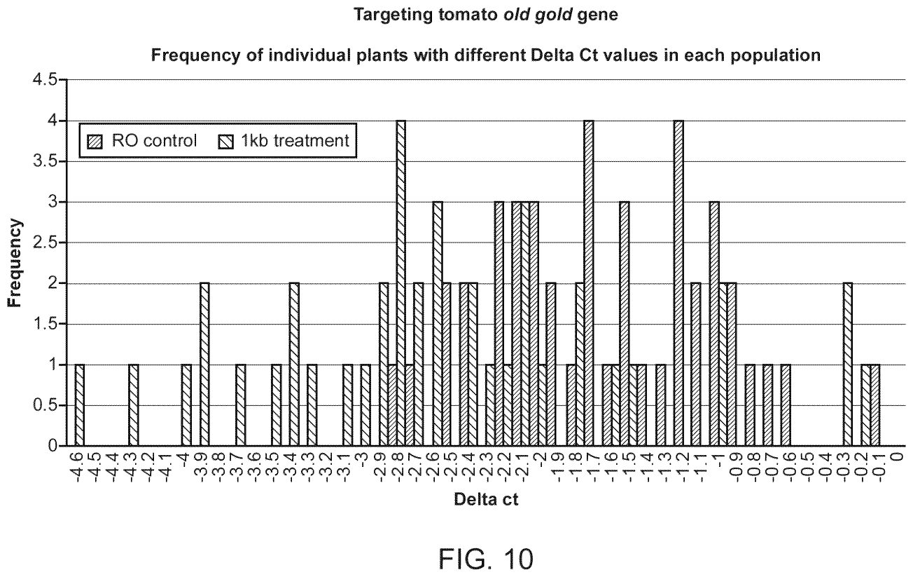

Threshold cycle (Ct) values were averaged from 3 technical replicates of each sample during RT-qPCR and used to calculate the ΔCt value (Old Gold target-GAPDH housekeeping reference). Changes in gene expression for treated (Old Gold) vs untreated (random oligo) populations are displayed as frequency histograms (FIG. 10). ΔCt values for each sample were rounded to 2 d.p. and sorted into groups with a class width of 0.1, ranging from 0 to ˜4.5 ΔCt (log2), with more negative ΔCt values indicating greater down-regulation of Old Gold target gene compared to GAPDH reference gene.

### Example 8—Examples of Sequence Modifications for Oligonucleotides

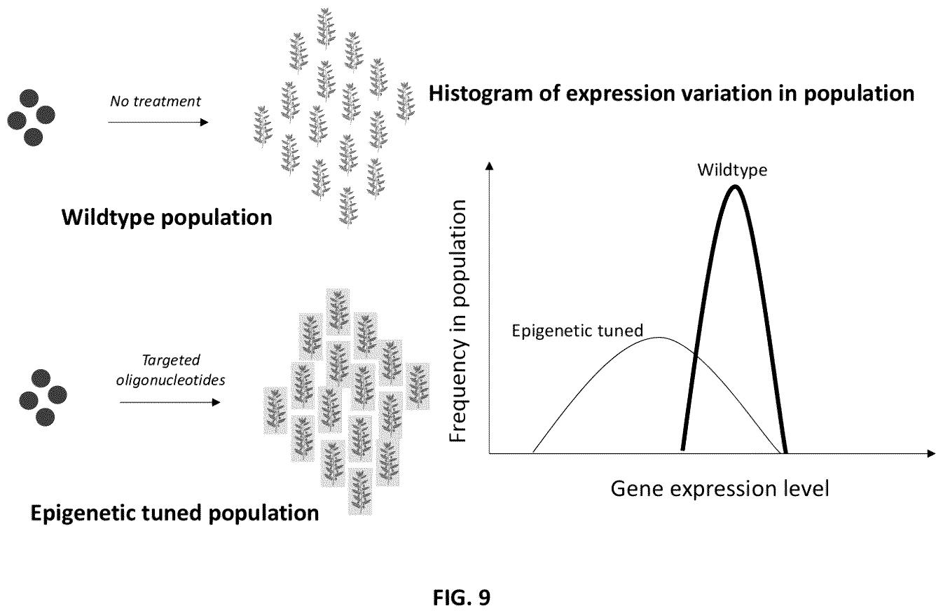

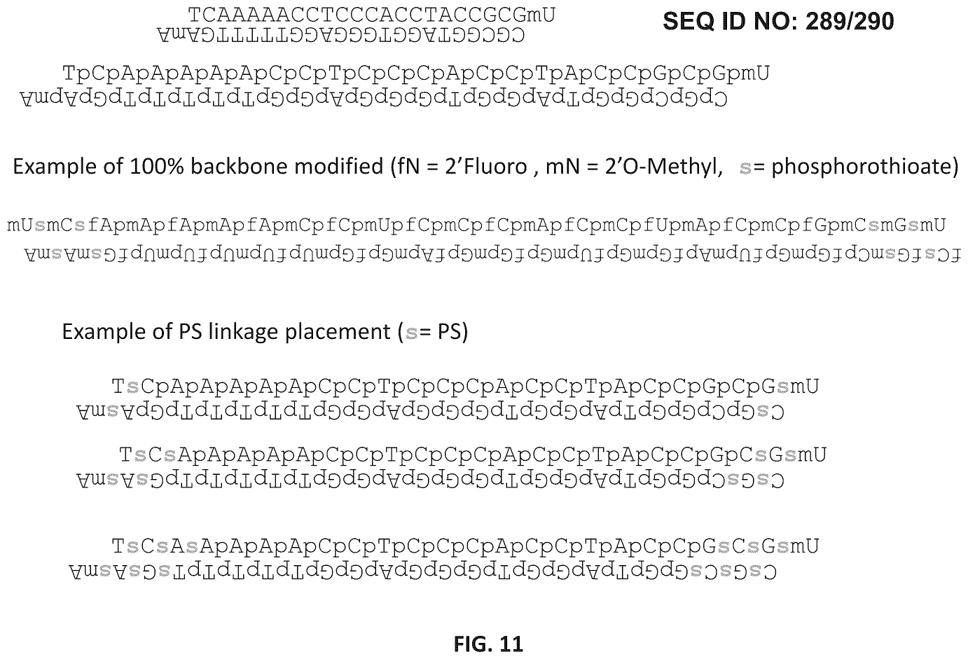

Nucleic acid constructs disclosed herein for example CATs oligonucleotides can have various modifications in individual or multiple locations. These modifications can include but are not limited to phosphorothioate modifications, 2′O-Methyl modifications, 2′Fluoro modifications, or any combination thereof. An example is shown in FIG. 11. An epigenetic modification using constructs herein such as CATs oligonucleotides can create populations with variable gene expression levels (FIG. 9).

Sequences
CATS oligonucleotide sequences for silencing PDS1
gene (mC = 2′-O-methyl cytosine, mG = 2′-O-
methyl guanine, mA = 2′-O-methyl adenine, mT =
2′-O-methyl thymine, mU = 2′-O-methyl uracil)

| | |
|---|---|
| SEQ ID NO: 1 | TGGCTACTATGTATTGATGTTAAmC |
| SEQ ID NO: 2 | TTAACATCAATACATAGTAGCCAmG |
| SEQ ID NO: 3 | CCATTAAGATGTACTCGCTCTGTmA |
| SEQ ID NO: 4 | ACAGAGCGAGTACATCTTAATGGmA |
| SEQ ID NO: 5 | TAATCATTTTTGTCTGTTATTTTmU |
| SEQ ID NO: 6 | AAAATAACAGACAAAAATGATTAmC |
| SEQ ID NO: 7 | GTCTATATTCAGACAGATGATAAmU |
| SEQ ID NO: 8 | TTATCATCTGTCTGAATATAGACmA |
| SEQ ID NO: 9 | CTAGACACATATACCAAGTAATGmA |
| SEQ ID NO: 10 | CATTACTTGGTATATGTGTCTAGmA |
| SEQ ID NO: 11 | GGATATAGGGAGTATGAACAACTmG |
| SEQ ID NO: 12 | AGTTGTTCATACTCCCTATATCCmU |
| SEQ ID NO: 13 | CAATTAGTTCCGTATTGATAATAmU |
| SEQ ID NO: 14 | TATTATCAATACGGAACTAATTGmU |
| SEQ ID NO: 15 | GGATCGACGTATTTATAATAATAmC |
| SEQ ID NO: 16 | TATTATTATAAATACGTCGATCCmA |
| SEQ ID NO: 17 | TGTTCTATATCTATATTTAATTAmU |
| SEQ ID NO: 18 | TAATTAAATATAGATATAGAACAmG |
| SEQ ID NO: 19 | GCGGAGGTCTCCACTCTTCTCTCmU |
| SEQ ID NO: 20 | GAGAGAAGAGTGGAGACCTCCGCmA |
| SEQ ID NO: 21 | CCATCTTATCATCGCCCACGTACmA |
| SEQ ID NO: 22 | GTACGTGGGCGATGATAAGATGGmA |
| SEQ ID NO: 23 | CCCAATTCCTCGCAACTGGGCTCmC |
| SEQ ID NO: 24 | GAGCCCAGTTGCGAGGAATTGGGmU |
| SEQ ID NO: 25 | CGCCTCCACGACACTGCCCCCCGmC |
| SEQ ID NO: 26 | CGGGGGGCAGTGTCGTGGAGGCGmG |
| SEQ ID NO: 27 | CAAGTCCGCCGCCTCCATTCTTCmA |
| SEQ ID NO: 28 | GAAGAATGGAGGCGGCGGACTTGmA |
| SEQ ID NO: 29 | GTGCGTTGGTGGGTCTGAAACAATmA |
| SEQ ID NO: 30 | ATTGTTTCAGACCCACCAACGCACmA |
| SEQ ID NO: 31 | GTCTCAAAAGGAGGTGAGCTGGGmA |
| SEQ ID NO: 32 | CCCAGCTCACCTCCTTTTGAGACmG |
| SEQ ID NO: 33 | TTTAGCCGACTAATTTTAGATGAmG |
| SEQ ID NO: 34 | TCATCTAAAATTAGTCGGCTAAAmU |
| SEQ ID NO: 35 | TAATCTTTAGTTCCGTGCCCCCGmC |
| SEQ ID NO: 36 | CGGGGGCACGGAACTAAAGATTAmU |
| SEQ ID NO: 37 | TGCAGGCTCACCCCGACGTGCCCmC |
| SEQ ID NO: 38 | GGGCACGTCGGGGTGAGCCTGCAmC |
| SEQ ID NO: 39 | GCCCTCCGCGCGCGATGCCCCCAmU |
| SEQ ID NO: 40 | TGGGGGCATCGCGCGCGGAGGGCmA |
| SEQ ID NO: 41 | CACCGACGCAGAGCTCGCCCATGmC |
| SEQ ID NO: 42 | CATGGGCGAGCTCTGCGTCGGTGmU |
| SEQ ID NO: 43 | CACGTGCCCCGGCGGCGTCGCGAmU |
| SEQ ID NO: 44 | TCGCGACGCCGCCGGGGCACGTGmC |
| SEQ ID NO: 45 | TATCCCCGCCGTCGCGCGCCTACmG |
| SEQ ID NO: 46 | GTAGGCGCGCGACGGCGGGGATAmC |
| SEQ ID NO: 47 | GTACCTGGAGCAGAAGCGATTTCmG |
| SEQ ID NO: 48 | GAAATCGCTTCTGCTCCAGGTACmA |
| SEQ ID NO: 49 | TGCCGAGGAGCTCACCGCTGTGAmA |
| SEQ ID NO: 50 | TCACAGCGGTGAGCTCCTCGGCAmA |
| SEQ ID NO: 51 | TAGTCCCGTCCCCTCCAGGTCCTmG |
| SEQ ID NO: 52 | AGGACCTGGAGGGGACGGGACTAmA |
| SEQ ID NO: 53 | AATTCACTTGTGTATTCCCCCGAmA |
| SEQ ID NO: 54 | TCGGGGGAATACACAAGTGAATTmG |
| SEQ ID NO: 55 | GAGACAGTCTGCACGGCTCCTATmC |
| SEQ ID NO: 56 | ATAGGAGCCGTGCAGACTGTCTCmG |
| SEQ ID NO: 57 | GCATGTACCCAATACCCTGTTTTmG |
| SEQ ID NO: 58 | AAAACAGGGTATTGGGTACATGCmU |
| SEQ ID NO: 59 | TGACCTTGGGTACTATTATAGCAmC |
| SEQ ID NO: 60 | TGCTATAATAGTACCCAAGGTCAmA |
| SEQ ID NO: 61 | TTTCAGTTCGAGTGTTAGACCCTmG |
| SEQ ID NO: 62 | AGGGTCTAACACTCGAACTGAAAmC |

CATS oligonucleotide sequences for silencing LZY1
gene (mC = 2′-O-methyl cytosine, mG = 2′-O-methyl
guanine, mA = 2′-O-methyl adenine, mT =
2′-O-methyl thymine, mU = 2′-O-methyl uracil)

| | |
|---|---|
| SEQ ID NO: 63 | CTTTTTGGAATTCTTCTGACCAGmG |
| SEQ ID NO: 64 | CTGGTCAGAAGAATTCCAAAAAGmA |
| SEQ ID NO: 65 | CAAGAAAATTAAGGATATATAGCmG |
| SEQ ID NO: 66 | GCTATATATCCTTAATTTTCTTGmG |
| SEQ ID NO: 67 | TAAGGCATGTCGATAATAAAGACmU |
| SEQ ID NO: 68 | GTCTTTATTATCGACATGCCTTAmG |
| SEQ ID NO: 69 | ATAGGACCGAGCAACTCGGATTCmG |
| SEQ ID NO: 70 | GAATCCGAGTTGCTCGGTCCTATmU |
| SEQ ID NO: 71 | GACTCGGACTTGGACTGTGCAACmU |
| SEQ ID NO: 72 | GTTGCACAGTCCAAGTCCGAGTCmA |
| SEQ ID NO: 73 | AGCCGGGATACTCAAAATCGAAAmU |
| SEQ ID NO: 74 | TTTCGATTTTGAGTATCCCGGCTmG |
| SEQ ID NO: 75 | ACTCGGTCCATAGGTTAATACTCmA |
| SEQ ID NO: 76 | GAGTATTAACCTATGGACCGAGTmC |
| SEQ ID NO: 77 | TCATATTTTACATCTAAACGTTCmG |
| SEQ ID NO: 78 | GAACGTTTAGATGTAAAATATGAmG |
| SEQ ID NO: 79 | TGGAAGCAAACACCCCCTTAGACmG |
| SEQ ID NO: 80 | GTCTAAGGGGGTGTTTGCTTCCAmC |
| SEQ ID NO: 81 | CCACCTCTTGTCGACCATTTGTAmG |
| SEQ ID NO: 82 | TACAAATGGTCGACAAGAGGTGGmU |
| SEQ ID NO: 83 | CATGTTCCGTTCTGCCGACTGATmG |
| SEQ ID NO: 84 | ATCAGTCGGCAGAACGGAACATGmA |
| SEQ ID NO: 85 | CCTGCAGCTGCAGCTGCAGTGCGmU |
| SEQ ID NO: 86 | CGCACTGCAGCTGCAGCTGCAGGmA |
| SEQ ID NO: 87 | ACTCCATCGTCTATTAATGGCTCmG |
| SEQ ID NO: 88 | GAGCCATTAATAGACGATGGAGTmA |
| SEQ ID NO: 89 | CCAGCGCTCGGCTTAGACAAGCCmU |
| SEQ ID NO: 90 | GGCTTGTCTAAGCCGAGCGCTGGmA |
| SEQ ID NO: 91 | ATGAAGGTCAGTCAGTAGTCCCAmC |
| SEQ ID NO: 92 | TGGGACTACTGACTGACCTTCATmG |
| SEQ ID NO: 93 | AGCTTTTAGTCTAGCTCGACAGTmC |
| SEQ ID NO: 94 | ACTGTCGAGCTAGACTAAAAGCTmG |
| SEQ ID NO: 95 | TTCTTTCTAATCCACCTATTTTCmU |
| SEQ ID NO: 96 | GAAAATAGGTGGATTAGAAAGAAmC |
| SEQ ID NO: 97 | GCGACTCACTCTCGTAGTTGGTGmU |
| SEQ ID NO: 98 | CACCAACTACGAGAGTGAGTCGCmC |
| SEQ ID NO: 99 | TCATGACCTCCTCCTCAAGCTCGmG |
| SEQ ID NO: 100 | CGAGCTTGAGGAGGAGGTCATGAmA |
| SEQ ID NO: 101 | CTCATATTTGAAGCCTCCTTGTTmC |
| SEQ ID NO: 102 | AACAAGGAGGCTTCAAATATGAGmA |
| SEQ ID NO: 103 | AGAAAGCATCGACCTTAGCAAGGmU |
| SEQ ID NO: 104 | CCTTGCTAAGGTCGATGCTTTCTmU |
| SEQ ID NO: 105 | TTGTCCATGCGCTTGGTGAGGTCmG |
| SEQ ID NO: 106 | GACCTCACCAAGCGCATGGACAAmG |
| SEQ ID NO: 107 | AATCTAACCTTTGAGTACCAAATmG |
| SEQ ID NO: 108 | ATTTGGTACTCAAAGGTTAGATTmG |
| SEQ ID NO: 109 | TGGAGACACATACACAGAGAAGAmA |
| SEQ ID NO: 110 | TCTTCTCTGTGTATGTGTCTCCAmU |
| SEQ ID NO: 111 | CTTGTTTGGATTGAAACCATTACmA |
| SEQ ID NO: 112 | GTAATGGTTTCAATCCAAACAAGmA |
| SEQ ID NO: 113 | ATATATTGGACTTGTATTCCAAGmC |
| SEQ ID NO: 114 | CTTGGAATACAAGTCCAATATATmA |
| SEQ ID NO: 115 | GTCCTTATAGATTTGGACACTTAmU |
| SEQ ID NO: 116 | TAAGTGTCCAAATCTATAAGGACmU |
| SEQ ID NO: 117 | CAAATCTTCTTGCCTAAGCAAATmU |
| SEQ ID NO: 118 | ATTTGCTTAGGCAAGAAGATTTGmU |
| SEQ ID NO: 119 | CTAAACTCTATTTTATACTCCCTmC |
| SEQ ID NO: 120 | AGGGAGTATAAAATAGAGTTTAGmU |
| SEQ ID NO: 121 | AGTGTTCATTTTGGCTCCTCATTmU |
| SEQ ID NO: 122 | AATGAGGAGCCAAAATGAACACTmA |
| SEQ ID NO: 123 | TCAGATGGATGAAAATGAATCTAmG |
| SEQ ID NO: 124 | TAGATTCATTTTCATCCATCTGAmA |
| SEQ ID NO: 125 | TGAATCCACTGATATGTTAAAACmG |
| SEQ ID NO: 126 | GTTTTAACATATCAGTGGATTCAmU |
| SEQ ID NO: 127 | GGGACGGAGAGAGTATATTCCAAmG |
| SEQ ID NO: 128 | TTGGAATATACTCTCTCCGTCCCmA |
| SEQ ID NO: 129 | CTATCTTTGGGTTTTCATCTTTTmU |
| SEQ ID NO: 130 | AAAAGATGAAAACCCAAAGATAGmC |
| SEQ ID NO: 131 | GACCAGGAGGGACTCTATTTATAmU |
| SEQ ID NO: 132 | TATAAATAGAGTCCCTCCTGGTCmA |
| SEQ ID NO: 133 | GATAATAAAGACTCTGACTAATAmG |
| SEQ ID NO: 134 | TATTAGTCAGAGTCTTTATTATCmG |
| SEQ ID NO: 135 | AGACTCTGACTAATAGGACCGAGmC |
| SEQ ID NO: 136 | CTCGGTCCTATTAGTCAGAGTCTmU |
| SEQ ID NO: 137 | ACTAATAGGACCGAGCAACTCGGmA |
| SEQ ID NO: 138 | CCGAGTTGCTCGGTCCTATTAGTmC |
| SEQ ID NO: 139 | GACCGAGCAACTCGGATTCGGTGmG |
| SEQ ID NO: 140 | CACCGAATCCGAGTTGCTCGGTCmC |
| SEQ ID NO: 141 | AACTCGGATTCGGTGGAGTGACTmC |
| SEQ ID NO: 142 | AGTCACTCCACCGAATCCGAGTTmG |
| SEQ ID NO: 143 | TTCGGTGGAGTGACTCGGACTTGmG |
| SEQ ID NO: 144 | CAAGTCCGAGTCACTCCACCGAAmU |
| SEQ ID NO: 145 | AGTGACTCGGACTTGGACTGTGCmA |
| SEQ ID NO: 146 | GCACAGTCCAAGTCCGAGTCACTmC |
| SEQ ID NO: 147 | GGACTTGGACTGTGCAACTCGGAmU |
| SEQ ID NO: 148 | TCCGAGTTGCACAGTCCAAGTCCmG |
| SEQ ID NO: 149 | ACTGTGCAACTCGGATTCAGCCGmG |
| SEQ ID NO: 150 | CGGCTGAATCCGAGTTGCACAGTmC |
| SEQ ID NO: 151 | ACTCGGATTCAGCCGGGATACTCmA |
| SEQ ID NO: 152 | GAGTATCCCGGCTGAATCCGAGTmU |
| SEQ ID NO: 153 | TCAGCCGGGATACTCAAAATCGAmA |
| SEQ ID NO: 154 | TCGATTTTGAGTATCCCGGCTGAmA |
| SEQ ID NO: 155 | GATACTCAAAATCGAAATCCAAGmG |
| SEQ ID NO: 156 | CTTGGATTTCGATTTTGAGTATCmC |
| SEQ ID NO: 157 | AAATCGAAATCCAAGGGACTCGGmU |
| SEQ ID NO: 158 | CCGAGTCCCTTGGATTTCGATTTmU |
| SEQ ID NO: 159 | ATCCAAGGGACTCGGTCCATAGGmU |
| SEQ ID NO: 160 | CCTATGGACCGAGTCCCTTGGATmU |
| SEQ ID NO: 161 | GACTCGGTCCATAGGTTAATACTmC |
| SEQ ID NO: 162 | AGTATTAACCTATGGACCGAGTCmC |
| SEQ ID NO: 163 | TCATATTTTACATCTAAACGTTCmG |
| SEQ ID NO: 164 | GAACGTTTAGATGTAAAATATGAmG |
| SEQ ID NO: 165 | TACATCTAAACGTTCGATGTGCGmU |
| SEQ ID NO: 166 | CGCACATCGAACGTTTAGATGTAmA |
| SEQ ID NO: 167 | AACGTTCGATGTGCGTGGAAGCAmA |
| SEQ ID NO: 168 | TGCTTCCACGCACATCGAACGTTmU |
| SEQ ID NO: 169 | ATGTGCGTGGAAGCAAACACCCCmC |
| SEQ ID NO: 170 | GGGGTGTTTGCTTCCACGCACATmC |
| SEQ ID NO: 171 | GGAAGCAAACACCCCCTTAGACGmU |
| SEQ ID NO: 172 | CGTCTAAGGGGGTGTTTGCTTCCmA |
| SEQ ID NO: 173 | ACACCCCCTTAGACGTGGGACACmC |
| SEQ ID NO: 174 | GTGTCCCACGTCTAAGGGGGTGTmU |
| SEQ ID NO: 175 | TTAGACGTGGGACACCACCTCTTmG |
| SEQ ID NO: 176 | AAGAGGTGGTGTCCCACGTCTAAmG |
| SEQ ID NO: 177 | GGGACACCACCTCTTGTCGACCAmU |
| SEQ ID NO: 178 | TGGTCGACAAGAGGTGGTGTCCCmA |
| SEQ ID NO: 179 | ACCTCTTGTCGACCATTTGTAGCmC |
| SEQ ID NO: 180 | GCTACAAATGGTCGACAAGAGGTmG |
| SEQ ID NO: 181 | TCGACCATTTGTAGCCTTCTTCAmU |
| SEQ ID NO: 182 | TGAAGAAGGCTACAAATGGTCGAmC |
| SEQ ID NO: 183 | TTGTAGCCTTCTTCATGTTCCGTmU |
| SEQ ID NO: 184 | ACGGAACATGAAGAAGGCTACAAmA |
| SEQ ID NO: 185 | TTCTTCATGTTCCGTTCTGCCGAmC |
| SEQ ID NO: 186 | TCGGCAGAACGGAACATGAAGAAmG |
| SEQ ID NO: 187 | GTTCCGTTCTGCCGACTGATGGAmU |
| SEQ ID NO: 188 | TCCATCAGTCGGCAGAACGGAACmA |
| SEQ ID NO: 189 | CTGCCGACTGATGGATCACTCCTmG |
| SEQ ID NO: 190 | AGGAGTGATCCATCAGTCGGCAGmA |
| SEQ ID NO: 191 | TGATGGATCACTCCTGCAGCTGCmA |
| SEQ ID NO: 192 | GCAGCTGCAGGAGTGATCCATCAmG |
| SEQ ID NO: 193 | CACTCCTGCAGCTGCAGCTGCAGmU |
| SEQ ID NO: 194 | CTGCAGCTGCAGCTGCAGGAGTGmA |

CATS oligonucleotide sequences for silencing
PPO gene (mC = 2′-O-methyl cytosine, mG = 2′-O-
methyl guanine, mA = 2′-O-methyl adenine, mT =
2′-O-methyl thymine, mU = 2′-O-methyl uracil)

| | |
|---|---|
| SEQ ID NO: 195 | ATGAAGCAAAACTCTAAAGTTGAmC |
| SEQ ID NO: 196 | TCAACTTTAGAGTTTTGCTTCATmA |
| SEQ ID NO: 197 | AACCCAGTTTTTCAGCTCTCACTmA |
| SEQ ID NO: 198 | AGTGAGAGCTGAAAAACTGGGTTmG |
| SEQ ID NO: 199 | ATTACCAATTGATCATCATCTTGmC |
| SEQ ID NO: 200 | CAAGATGATGATCAATTGGTAATmU |
| SEQ ID NO: 201 | ATTACAACTTTCCAGCTATTTTGmC |
| SEQ ID NO: 202 | CAAAATAGCTGGAAAGTTGTAATmA |
| SEQ ID NO: 203 | CACTTTATAATCCTAATCCTACAmC |
| SEQ ID NO: 204 | TGTAGGATTAGGATTATAAAGTGmU |
| SEQ ID NO: 205 | AAGCTCATTCAACAACACAATTAmG |
| SEQ ID NO: 206 | TAATTGTGTTGTTGAATGAGCTTmU |
| SEQ ID NO: 207 | TCAAACACAAAATAGAGTTATGGmC |
| SEQ ID NO: 208 | CCATAACTCTATTTTGTGTTTGAmU |
| SEQ ID NO: 209 | TTAACTTCATGTACTACCATTTCmC |
| SEQ ID NO: 210 | GAAATGGTAGTACATGAAGTTAAmA |
| SEQ ID NO: 211 | CATCCAAAATTTTCGTCCGTCCAmA |
| SEQ ID NO: 212 | TGGACGGACGAAAATTTTGGATGmG |
| SEQ ID NO: 213 | CGATAACTTTAAGGTGAATTGTGmA |
| SEQ ID NO: 214 | CACAATTCACCTTAAAGTTATCGmA |
| SEQ ID NO: 215 | TAATGAAGGAAAATCTTTTCCAGmG |
| SEQ ID NO: 216 | CTGGAAAAGATTTTCCTTCATTAmU |
| SEQ ID NO: 217 | GATAGACGAAATGTCCTCCTTGGmU |
| SEQ ID NO: 218 | CCAAGGAGGACATTTCGTCTATCmA |
| SEQ ID NO: 219 | GGCTCTATGGAGCATCTAATCTTmA |
| SEQ ID NO: 220 | AAGATTAGATGCTCCATAGAGCCmC |
| SEQ ID NO: 221 | AACCAACGAGCCATTTGCCCTAGmG |
| SEQ ID NO: 222 | CTAGGGCAAATGGCTCGTTGGTTmA |
| SEQ ID NO: 223 | GTACCACCCCCAGACTTCTCAACmA |
| SEQ ID NO: 224 | GTTGAGAAGTCTGGGGGTGGTACmC |
| SEQ ID NO: 225 | TCTGTCAACCGACCCAACCGACCmC |
| SEQ ID NO: 226 | GGTCGGTTGGGTCGGTTGACAGAmA |
| SEQ ID NO: 227 | TCCAGACTGTAAGTTATTTTTCTmG |
| SEQ ID NO: 228 | AGAAAAATAACTTACAGTCTGGAmU |
| SEQ ID NO: 229 | CAGAGCTTCTAAGAACAAAAACTmU |
| SEQ ID NO: 230 | AGTTTTTGTTCTTAGAAGCTCTGmU |
| SEQ ID NO: 231 | GTATGGCTGCTATACAAAATTCCmC |
| SEQ ID NO: 232 | GGAATTTTGTATAGCAGCCATACmA |
| SEQ ID NO: 233 | CGCTTCCTGGAATAATTGATATGmG |
| SEQ ID NO: 234 | CATATCAATTATTCCAGGAAGCGmG |
| SEQ ID NO: 235 | TATTATATAAGGCAAGGTATAGCmC |
| SEQ ID NO: 236 | GCTATACCTTGCCTTATATAATAmA |
| SEQ ID NO: 237 | TCATTCAAAACCTAGCAATAATGmG |
| SEQ ID NO: 238 | CATTATTGCTAGGTTTTGAATGAmA |
| SEQ ID NO: 239 | GTAGTAATACATCTCTCAAAACTmC |
| SEQ ID NO: 240 | AGTTTTGAGAGATGTATTACTACmU |
| SEQ ID NO: 241 | TTCTTCCTCCACTTCTTTATCTTmC |
| SEQ ID NO: 242 | AAGATAAAGAAGTGGAGGAAGAAmG |
| SEQ ID NO: 243 | AAGCCCTCTTCAACTTTTCATCCmA |
| SEQ ID NO: 244 | GGATGAAAAGTTGAAGAGGGCTTmA |
| SEQ ID NO: 245 | CGTACCAAATGTTCAAAGTTTCAmU |
| SEQ ID NO: 246 | TGAAACTTTGAACATTTGGTACGmU |
| SEQ ID NO: 247 | TACCAATAATAACGGTGACCAAAmA |
| SEQ ID NO: 248 | TTTGGTCACCGTTATTATTGGTAmA |
| SEQ ID NO: 249 | GTTGAAACGAATTCTGTTGATCGmA |
| SEQ ID NO: 250 | CGATCAACAGAATTCGTTTCAACmG |
| SEQ ID NO: 251 | TTCTTCTTGGCTTAGGTGGTCTTmU |
| SEQ ID NO: 252 | AAGACCACCTAAGCCAAGAAGAAmC |
| SEQ ID NO: 253 | TGCTAATGCTATACCATTAGCTGmC |
| SEQ ID NO: 254 | CAGCTAATGGTATAGCATTAGCAmA |
| SEQ ID NO: 255 | AATTATCAATGCTTGTGAATCTCmG |
| SEQ ID NO: 256 | GAGATTCACAAGCATTGATAATTmU |
| SEQ ID NO: 257 | TGTTAAAAAATTTCCTCACCTACmC |
| SEQ ID NO: 258 | GTAGGTGAGGAAATTTTTTAACAmA |
| SEQ ID NO: 259 | GAATTGTTCGATATGAGATCGAGmC |
| SEQ ID NO: 260 | CTCGATCTCATATCGAACAATTCmG |
| SEQ ID NO: 261 | TGGAGTAATATTTTATTTGGCTCmC |
| SEQ ID NO: 262 | GAGCCAAATAAAATATTACTCCAmU |
| SEQ ID NO: 263 | TATATAAGGCAATGTATAGCCCTmA |
| SEQ ID NO: 264 | AGGGCTATACATTGCCTTATATAmA |
| SEQ ID NO: 265 | TCATCCAAAAACTAGCAATGGCAmA |
| SEQ ID NO: 266 | TGCCATTGCTAGTTTTTGGATGAmA |
| SEQ ID NO: 267 | TAATAGTAGTAGTACCACTCTCAmA |
| SEQ ID NO: 268 | TGAGAGTGGTACTACTACTATTAmC |
| SEQ ID NO: 269 | TTTACTTCTTCCTCCACTTCTTTmA |
| SEQ ID NO: 270 | AAAGAAGTGGAGGAAGAAGTAAAmA |
| SEQ ID NO: 271 | CTCCTAAGCCCTCTCAACTTTTCmC |
| SEQ ID NO: 272 | GAAAAGTTGAGAGGGCTTAGGAGmU |
| SEQ ID NO: 273 | ACGTAACAAAACGTTCAAAGTTTmC |
| SEQ ID NO: 274 | AAACTTTGAACGTTTTGTTACGTmU |
| SEQ ID NO: 275 | GTTACCAATAATAACGGTGACCAmA |
| SEQ ID NO: 276 | TGGTCACCGTTATTATTGGTAACmC |
| SEQ ID NO: 277 | ACGTTGAAACGAATTCTGTTGATmC |
| SEQ ID NO: 278 | ATCAACAGAATTCGTTTCAACGTmU |
| SEQ ID NO: 279 | TGTTCTTCTTGGTTTAGGAGGTCmU |
| SEQ ID NO: 280 | GACCTCCTAAACCAAGAAGAACAmU |
| SEQ ID NO: 281 | GTTGCTAATGCTATACCATTAGCmU |
| SEQ ID NO: 282 | GCTAATGGTATAGCATTAGCAACmA |
| SEQ ID NO: 283 | CTTCTCCAACTCCACCTCCTGATmC |
| SEQ ID NO: 284 | ATCAGGAGGTGGAGTTGGAGAAGmC |
| SEQ ID NO: 285 | CCAAACAATCTGTTCAGCTATTCmA |
| SEQ ID NO: 286 | GAATAGCTGAACAGATTGTTTGGmU |
| SEQ ID NO: 287 | AAATGTGTAAAAGATTTCCCACAmC |
| SEQ ID NO: 288 | TGTGGGAAATCTTTTACACATTTmU |
| SEQ ID NO: 289 | TCAAAAACCTCCCACCTACCGCGmU |
| SEQ ID NO: 290 | CGCGGTAGGTGGGAGGTTTTTGAmA |
| SEQ ID NO: 291 | TGTTGGAGTGGTAGGTGAGCCTCmU |
| SEQ ID NO: 292 | GAGGCTCACCTACCACTCCAACAmU |
| SEQ ID NO: 293 | AGCAACATACTATATAATGCAAGmG |
| SEQ ID NO: 294 | CTTGCATTATATAGTATGTTGCTmA |
| SEQ ID NO: 295 | TATGAATCTTCATCAACCAAAAGmC |
| SEQ ID NO: 296 | CTTTTGGTTGATGAAGATTCATAmU |
| SEQ ID NO: 297 | AATGGCAAGTGTGTGCAATAGTAmG |
| SEQ ID NO: 298 | TACTATTGCACACACTTGCCATTmG |
| SEQ ID NO: 299 | ACTACAACTCTCAAAACTCCTTTmC |
| SEQ ID NO: 300 | AAAGGAGTTTTGAGAGTTGTAGTmA |
| SEQ ID NO: 301 | CCAATACTTCTTTATCTTCAACTmC |
| SEQ ID NO: 302 | AGTTGAAGATAAAGAAGTATTGGmA |
| SEQ ID NO: 303 | CTCTCAACTTTTCCTCCATGGAAmA |
| SEQ ID NO: 304 | TTCCATGGAGGAAAAGTTGAGAGmG |
| SEQ ID NO: 305 | CAAATGTTCAAAGTTTCATGCAAmG |
| SEQ ID NO: 306 | TTGCATGAAACTTTGAACATTTGmG |
| SEQ ID NO: 307 | ATAATAACGGTGACCAAAACGTTmG |
| SEQ ID NO: 308 | AACGTTTTGGTCACCGTTATTATmU |
| SEQ ID NO: 309 | TTCTGTTGATCGAAGAAATGTTCmU |
| SEQ ID NO: 310 | GAACATTTCTTCGATCAACAGAAmU |
| SEQ ID NO: 311 | TTAGGAGGTCTATATGGTGTTGCmU |
| SEQ ID NO: 312 | GCAACACCATATAGACCTCCTAAmA |
| SEQ ID NO: 313 | TACCATTAGCTGCATCCGCTGCTmC |
| SEQ ID NO: 314 | AGCAGCGGATGCAGCTAATGGTAmU |
| SEQ ID NO: 315 | GATCAAAGGATGGCTAAATTTTTmC |
| SEQ ID NO: 316 | AAAAATTTAGCCATCCTTTGATCmA |
| SEQ ID NO: 317 | TTGAACTTGAGGATCAATATTTCmC |
| SEQ ID NO: 318 | GAAATATTGATCCTCAAGTTCAAmA |
| SEQ ID NO: 319 | GAGAGTGAGTAATTACTCCAAGAmU |
| SEQ ID NO: 320 | TCTTGGAGTAATTACTCACTCTCmU |
| SEQ ID NO: 321 | ACAATTATCACCAACGTGTTACAmC |
| SEQ ID NO: 322 | TGTAACACGTTGGTGATAATTGTmA |
| SEQ ID NO: 323 | GCTACATATACCTTCACCATTTTmG |
| SEQ ID NO: 324 | AAAATGGTGAAGGTATATGTAGCmA |
| SEQ ID NO: 325 | GCAACTCTTCTAACAAAAAATCAmC |
| SEQ ID NO: 326 | TGATTTTTTGTTAGAAGAGTTGCmA |
| SEQ ID NO: 327 | AACACAATGTCTTCTTCTAGTACmU |
| SEQ ID NO: 328 | GTACTAGAAGAAGACATTGTGTTmG |
| SEQ ID NO: 329 | TTCCATTATGCACCAACAAATCCmC |
| SEQ ID NO: 330 | GGATTTGTTGGTGCATAATGGAAmG |
| SEQ ID NO: 331 | TTCCTTCACCACCAACAACTCATmC |
| SEQ ID NO: 332 | ATGAGTTGTTGGTGGTGAAGGAAmG |
| SEQ ID NO: 333 | TCAAAACCCTCTCAACTTTTCCTmC |
| SEQ ID NO: 334 | AGGAAAAGTTGAGAGGGTTTTGAmU |
| SEQ ID NO: 335 | GACGTAATCAAAGTTTCAAGGTTmU |
| SEQ ID NO: 336 | AACCTTGAAACTTTGATTACGTCmU |
| SEQ ID NO: 337 | CGTCAACAATAATGTTGGCGAGCmA |
| SEQ ID NO: 338 | GCTCGCCAACATTATTGTTGACGmU |
| SEQ ID NO: 339 | AACCTTGACGCTGTTGATAGGCGmA |
| SEQ ID NO: 340 | CGCCTATCAACAGCGTCAAGGTTmU |
| SEQ ID NO: 341 | TTTTAGGGTTAGGAGGTCTTTATmG |
| SEQ ID NO: 342 | ATAAAGACCTCCTAACCCTAAAAmG |
| SEQ ID NO: 343 | TAATCTTGCACCATTAGCCTCTGmC |
| SEQ ID NO: 344 | CAGAGGCTAATGGTGCAAGATTAmG |
| SEQ ID NO: 345 | TGCAAAAGAAAATAGGATCTGCAmU |
| SEQ ID NO: 346 | TGCAGATCCTATTTTCTTTTGCAmU |
| SEQ ID NO: 347 | ATACAAAACCATTTCAAAACTGCmG |
| SEQ ID NO: 348 | GCAGTTTTGAAATGGTTTTGTATmU |
| SEQ ID NO: 349 | CGTTTGTACTAGGTACATGAATTmU |
| SEQ ID NO: 350 | AATTCATGTACCTAGTACAAACGmU |
| SEQ ID NO: 351 | AAATACTGATGAAACGCTGCAAAmG |
| SEQ ID NO: 352 | TTTGCAGCGTTTCATCAGTATTTmA |
| SEQ ID NO: 353 | ACTCATCCCAGCAATGGCTTCTTmC |
| SEQ ID NO: 354 | AAGAAGCCATTGCTGGGATGAGTmG |
| SEQ ID NO: 355 | CCTTTGTGCACTACCAATATTCCmC |
| SEQ ID NO: 356 | GGAATATTGGTAGTGCACAAAGGmU |
| SEQ ID NO: 357 | TCTCCAATAATACCAACTCATCTmU |
| SEQ ID NO: 358 | AGATGAGTTGGTATTATTGGAGAmA |
| SEQ ID NO: 359 | AAAACCCTCTCAGCTTTTCCTCCmA |
| SEQ ID NO: 360 | GGAGGAAAAGCTGAGAGGGTTTTmG |
| SEQ ID NO: 361 | CGTAGTCAAAGTTTCAAGGTTTCmA |
| SEQ ID NO: 362 | GAAACCTTGAAACTTTGACTACGmC |
| SEQ ID NO: 363 | ATAGCGAGCATGACAAAAATAACmC |
| SEQ ID NO: 364 | GTTATTTTTGTCATGCTCGCTATmA |
| SEQ ID NO: 365 | CGATGCTGTTGATAGGAGAAATGmU |
| SEQ ID NO: 366 | CATTTCTCCTATCAACAGCATCGmU |
| SEQ ID NO: 367 | GGGTTAGGAGGTCTTTATGGTGCmU |
| SEQ ID NO: 368 | GCACCATAAAGACCTCCTAACCCmU |
| SEQ ID NO: 369 | TTGCACCATTAGCCACTGCTGCTmC |
| SEQ ID NO: 370 | AGCAGCAGTGGCTAATGGTGCAAmG |
| SEQ ID NO: 371 | ACCACCTGATTTGAAAACTTGTAmG |
| SEQ ID NO: 372 | TACAAGTTTTCAAATCAGGTGGTmG |
| SEQ ID NO: 373 | ACTGTAACTCCTGGTGGTCCAGCmA |
| SEQ ID NO: 374 | GCTGGACCACCAGGAGTTACAGTmG |
| SEQ ID NO: 375 | TATGTGCTCACGTGGACACATTAmC |
| SEQ ID NO: 376 | TAATGTGTCCACGTGAGCACATAmG |
| SEQ ID NO: 377 | GATGCAATATTTATGATGTTCACmG |
| SEQ ID NO: 378 | GTGAACATCATAAATATTGCATCmA |
| SEQ ID NO: 379 | ATTATATTCTCGACAAGTTGAACmG |
| SEQ ID NO: 380 | GTTCAACTTGTCGAGAATATAATmU |
| SEQ ID NO: 381 | GGAAATGATGGAGATTATTATGGmU |
| SEQ ID NO: 382 | CCATAATAATCTCCATCATTTCCmA |
| SEQ ID NO: 383 | TCTTCCTCACAAGGTAATTACAAmA |
| SEQ ID NO: 384 | TTGTAATTACCTTGTGAGGAAGAmA |
| SEQ ID NO: 385 | CCTTAGCTTGCTCCATATTATTCmU |
| SEQ ID NO: 386 | GAATAATATGGAGCAAGCTAAGGmA |
| SEQ ID NO: 387 | TGCTAGCCCTAGATGTTCATGAAmU |
| SEQ ID NO: 388 | TTCATGAACATCTAGGGCTAGCAmU |
| SEQ ID NO: 389 | CAAGCAAAAAAATGTCTTCCATTmC |
| SEQ ID NO: 390 | AATGGAAGACATTTTTTTGCTTGmU |
| SEQ ID NO: 391 | CACTACCAATACTCTCTCTTCTTmC |
| SEQ ID NO: 392 | AAGAAGAGAGAGTATTGGTAGTGmG |
| SEQ ID NO: 393 | ACCACTTTTTCCAACTTGCATTCmU |
| SEQ ID NO: 394 | GAATGCAAGTTGGAAAAAGTGGTmG |
| SEQ ID NO: 395 | GCAAAAACATCAAAAATTTCCTCmC |
| SEQ ID NO: 396 | GAGGAAATTTTTGATGTTTTTGCmA |
| SEQ ID NO: 397 | AGCATAATGTCCATCGTAATTTCmC |
| SEQ ID NO: 398 | GAAATTACGATGGACATTATGCTmU |
| SEQ ID NO: 399 | GCAAAACCATAGATGATAATAGTmC |
| SEQ ID NO: 400 | ACTATTATCATCTATGGTTTTGCmA |
| SEQ ID NO: 401 | CAATAACTCACCCATCGACATTTmC |
| SEQ ID NO: 402 | AAATGTCGATGGGTGAGTTATTGmU |
| SEQ ID NO: 403 | ACAATATGATCGATAGAAGAAACmG |
| SEQ ID NO: 404 | GTTTCTTCTATCGATCATATTGTmU |

CATS oligonucleotide sequences for silencing
DWF1 and BR2 genes (mC = 2′-O-methyl cytosine,
mG = 2′-O-methyl guanine, mA = 2′-O-methyl
adenine, mT = 2′-O-methyl thymine, mU =
2′-O-methyl uracil)

| | |
|---|---|
| SEQ ID NO: 405 | TCGGCCTGCCGAGCCTAGACAATmA |
| SEQ ID NO: 406 | ATTGTCTAGGCTCGGCAGGCCGAmC |
| SEQ ID NO: 407 | TCTCCTCTCGATGGGGTCTCTCCmC |
| SEQ ID NO: 408 | GGAGAGACCCCATCGAGAGGAGAmG |
| SEQ ID NO: 409 | ATCCAGGCGCCGCCGGTGACCTTmC |
| SEQ ID NO: 410 | AAGGTCACCGGCGGCGCCTGGATmC |
| SEQ ID NO: 411 | GCGCACGCGCGGATCATTCGTCCmC |
| SEQ ID NO: 412 | GGACGAATGATCCGCGCGTGCGCmA |
| SEQ ID NO: 413 | ACAGTCTGACACGTTAGATAGAGmA |
| SEQ ID NO: 414 | CTCTATCTAACGTGTCAGACTGTmC |
| SEQ ID NO: 415 | ATGCGCCAGGTCGTGGACCGTCCmC |
| SEQ ID NO: 416 | GGACGGTCCACGACCTGGCGCATmG |
| SEQ ID NO: 417 | AGAGAACACTGCCGTCGGTTTTTmA |
| SEQ ID NO: 418 | AAAAACCGACGGCAGTGTTCTCTmG |
| SEQ ID NO: 419 | TTGACGTTCGAAAAGATGTCAACmA |
| SEQ ID NO: 420 | GTTGACATCTTTTCGAACGTCAAmU |
| SEQ ID NO: 421 | AGTTTTTTTATACAACTGAGAGAmG |
| SEQ ID NO: 422 | TCTCTCAGTTGTATAAAAAAACTmC |
| SEQ ID NO: 423 | GAGTGAGTTAAATGGCAACAAACmA |
| SEQ ID NO: 424 | GTTTGTTGCCATTTAACTCACTCmG |
| SEQ ID NO: 425 | GGAAAAAAACTATGAGATGTCATmC |
| SEQ ID NO: 426 | ATGACATCTCATAGTTTTTTTCCmU |
| SEQ ID NO: 427 | ATGACGGTAAATAAATATGGATGmG |
| SEQ ID NO: 428 | CATCCATATTTATTTACCGTCATmA |
| SEQ ID NO: 429 | CTAAAACGAAAAGTGGCAAAACCmU |
| SEQ ID NO: 430 | GGTTTTGCCACTTTTCGTTTTAGmU |
| SEQ ID NO: 431 | GACGGGTGTCGCCGAGTGCAGCCmG |
| SEQ ID NO: 432 | GGCTGCACTCGGCGACACCCGTCmC |
| SEQ ID NO: 433 | ACCCCCACCGATGTCCTGAGATTmG |
| SEQ ID NO: 434 | AATCTCAGGACATCGGTGGGGGTmU |
| SEQ ID NO: 435 | AAGCCGCAGGCAGCATCTGCATCmU |
| SEQ ID NO: 436 | GATGCAGATGCTGCCTGCGGCTTmC |
| SEQ ID NO: 437 | CTCCGCTCCGCCTACTGCTGCTGmG |
| SEQ ID NO: 438 | CAGCAGCAGTAGGCGGAGCGGAGmG |
| SEQ ID NO: 439 | GGCGGAGAAGGAGGCCCTTGCGCmC |
| SEQ ID NO: 440 | GCGCAAGGGCCTCCTTCTCCGCCmU |
| SEQ ID NO: 441 | GCCGGATCAGAGCCGGTAAGACCmA |
| SEQ ID NO: 442 | GGTCTTACCGGCTCTGATCCGGCmC |
| SEQ ID NO: 443 | CGCTCCTCCTCGCTGGTTGCTTTmC |
| SEQ ID NO: 444 | AAAGCAACCAGCGAGGAGGAGCGmU |
| SEQ ID NO: 445 | CGCCGGTATTCCCAGTCCGTGTGmC |
| SEQ ID NO: 446 | CACACGGACTGGGAATACCGGCGmG |
| SEQ ID NO: 447 | GTCTGCTCCCGTCGCTGCCTAGAmU |
| SEQ ID NO: 448 | TCTAGGCAGCGACGGGAGCAGACmA |
| SEQ ID NO: 449 | GGATCTTTCGTGCATGGCGGCAGmA |
| SEQ ID NO: 450 | CTGCCGCCATGCACGAAAGATCCmU |
| SEQ ID NO: 451 | CCCCCCCCCCCCCCCCCCCCCCCmC |
| SEQ ID NO: 452 | GGGGGGGGGGGGGGGGGGGGGGGmG |
| SEQ ID NO: 453 | CGTGTATACGAGTTTTCTCTAGGmC |
| SEQ ID NO: 454 | CCTAGAGAAAACTCGTATACACGmG |
| SEQ ID NO: 455 | CATGTTGGATTGGATTGCGCTAGmU |
| SEQ ID NO: 456 | CTAGCGCAATCCAATCCAACATGmC |
| SEQ ID NO: 457 | GAGGTGCCGGCCGTACCCATCCTmC |
| SEQ ID NO: 458 | AGGATGGGTACGGCCGGCACCTCmU |
| SEQ ID NO: 459 | AGAAAAAAGGCCCAGTCATTTTTmG |
| SEQ ID NO: 460 | AAAAATGACTGGGCCTTTTTTCTmG |
| SEQ ID NO: 461 | ATTTATTTTTACAGCTGCCACATmG |
| SEQ ID NO: 462 | ATGTGGCAGCTGTAAAAATAAATmA |
| SEQ ID NO: 463 | TTTTTGTTGGGGTTTTACTACTAmC |
| SEQ ID NO: 464 | TAGTAGTAAAACCCCAACAAAAAmC |
| SEQ ID NO: 465 | AACTGTTTTGTCAAATACTGTAAmC |
| SEQ ID NO: 466 | TTACAGTATTTGACAAAACAGTTmC |
| SEQ ID NO: 467 | AAAGCTGTTTGTAGGAGTGAAGCmU |
| SEQ ID NO: 468 | GCTTCACTCCTACAAACAGCTTTmA |
| SEQ ID NO: 469 | AAACAGAACTTCATATTGTTCCAmG |
| SEQ ID NO: 470 | TGGAACAATATGAAGTTCTGTTTmA |
| SEQ ID NO: 471 | TTCCAACAAAAAAATTGCAATTCmG |
| SEQ ID NO: 472 | GAATTGCAATTTTTTTGTTGGAAmC |
| SEQ ID NO: 473 | GCTACCAGTACAGCGCTAGATGGmA |
| SEQ ID NO: 474 | CCATCTAGCGCTGTACTGGTAGCmC |
| SEQ ID NO: 475 | CGAACATGAAACGTTTACTTTTTmC |
| SEQ ID NO: 476 | AAAAAGTAAACGTTTCATGTTCGmC |
| SEQ ID NO: 477 | TGTTTGATGGATCACATTTATCTmC |
| SEQ ID NO: 478 | AGATAAATGTGATCCATCAAACAmU |
| SEQ ID NO: 479 | TGTTGGATACCGGTACTTTTTACmG |
| SEQ ID NO: 480 | GTAAAAAGTACCGGTATCCAACAmC |
| SEQ ID NO: 481 | GTACAGGGGCCACTGGCTATATAmU |
| SEQ ID NO: 482 | TATATAGCCAGTGGCCCCTGTACmA |
| SEQ ID NO: 483 | CACTCCATTAATTTCCAGGGATGmC |
| SEQ ID NO: 484 | CATCCCTGGAAATTAATGGAGTGmA |
| SEQ ID NO: 485 | CTCTCTCTGCTACATACATCCATmU |
| SEQ ID NO: 486 | ATGGATGTATGTAGCAGAGAGAGmA |
| SEQ ID NO: 487 | TTTTTTGTGGAATTTTGCACTTGmG |
| SEQ ID NO: 488 | CAAGTGCAAAATTCCACAAAAAAmA |
| SEQ ID NO: 489 | TCTCAGTTTAATTTGGAGGATCAmA |
| SEQ ID NO: 490 | TGATCCTCCAAATTAAACTGAGAmA |
| SEQ ID NO: 491 | AGACATACATGGCGGATCCTCTGmG |
| SEQ ID NO: 492 | CAGAGGATCCGCCATGTATGTCTmC |
| SEQ ID NO: 493 | AAGGTCTTAGCGGACTACTTGGTmG |
| SEQ ID NO: 494 | ACCAAGTAGTCCGCTAAGACCTTmC |
| SEQ ID NO: 495 | CTTCGTGGCCCTTCCAATATCTGmC |
| SEQ ID NO: 496 | CAGATATTGGAAGGGCCACGAAGmA |
| SEQ ID NO: 497 | TGGTGAACACGTGGTCCGCCATGmA |
| SEQ ID NO: 498 | CATGGCGGACCACGTGTTCACCAmG |
| SEQ ID NO: 499 | GAACACCAGGAGAACGTAGAGGGmU |
| SEQ ID NO: 500 | CCCTCTACGTTCTCCTGGTGTTCmC |
| SEQ ID NO: 501 | ATCCGAAGAGGGACGGCCTCGTCmU |
| SEQ ID NO: 502 | GACGAGGCCGTCCCTCTTCGGATmC |
| SEQ ID NO: 503 | GTCGTGGGCATGCGCAACGTGGAmC |
| SEQ ID NO: 504 | TCCACGTTGCGCATGCCCACGACmC |
| SEQ ID NO: 505 | GGTCGACCTCTCCGCGTTGAGGAmA |
| SEQ ID NO: 506 | TCCTCAACGCGGAGAGGTCGACCmU |
| SEQ ID NO: 507 | GGATGGTCGCCAGGGTGGAGCCTmC |
| SEQ ID NO: 508 | AGGCTCCACCCTGGCGACCATCCmU |
| SEQ ID NO: 509 | AAGGCTACCTGCCCCATGAACCTmC |
| SEQ ID NO: 510 | AGGTTCATGGGGCAGGTAGCCTTmG |
| SEQ ID NO: 511 | GGACGACCTTACCGTCGGTGGTCmU |
| SEQ ID NO: 512 | GACCACCGACGGTAAGGTCGTCCmA |
| SEQ ID NO: 513 | GGAGCTCTCACGTCTACGGCCTCmU |
| SEQ ID NO: 514 | GAGGCCGTAGACGTGAGAGCTCCmC |
| SEQ ID NO: 515 | GAGGTCGTTCTTGCGGATGGGCAmG |
| SEQ ID NO: 516 | TGCCCATCCGCAAGAACGACCTCmC |
| SEQ ID NO: 517 | CGAGCACTCCGACCTCTTCTATGmG |
| SEQ ID NO: 518 | CATAGAAGAGGTCGGAGTGCTCGmU |
| SEQ ID NO: 519 | TCGGGCTCCTGGTTTCGGCTGAGmA |
| SEQ ID NO: 520 | CTCAGCCGAAACCAGGAGCCCGAmU |
| SEQ ID NO: 521 | TACATGAGGCTCACGTACACTCCmU |
| SEQ ID NO: 522 | GGAGTGTACGTGAGCCTCATGTAmC |
| SEQ ID NO: 523 | CGCGGAGGCTTATGCTGATTCGTmU |
| SEQ ID NO: 524 | ACGAATCAGCATAAGCCTCCGCGmA |
| SEQ ID NO: 525 | CACATGAACCCGTATCGCGAGATmG |
| SEQ ID NO: 526 | ATCTCGCGATACGGGTTCATGTGmU |
| SEQ ID NO: 527 | TCTTTATCACAGTGGATGCATATmG |
| SEQ ID NO: 528 | ATATGCATCCACTGTGATAAAGAmA |
| SEQ ID NO: 529 | ACAGATGGTTAGCGAGTGACAGTmA |
| SEQ ID NO: 530 | ACTGTCACTCGCTAACCATCTGTmU |
| SEQ ID NO: 531 | AGTTGTCCGACACTTCATCGGTAmA |
| SEQ ID NO: 532 | TACCGATGAAGTGTCGGACAACTmU |
| SEQ ID NO: 533 | ACCGAGTGAATGGAAGAAAAACGmA |
| SEQ ID NO: 534 | CGTTTTTCTTCCATTCACTCGGTmU |
| SEQ ID NO: 535 | ACAGCAGGTTTTCTTAAAAAACGmU |
| SEQ ID NO: 536 | CGTTTTTTAAGAAAACCTGCTGTmG |
| SEQ ID NO: 537 | TTAAGAAGAGACCAAAATATGGTmC |
| SEQ ID NO: 538 | ACCATATTTTGGTCTCTTCTTAAmU |
| SEQ ID NO: 539 | TTCTAAACATTAGTTCTCATCACmC |
| SEQ ID NO: 540 | GTGATGAGAACTAATGTTTAGAAmA |
| SEQ ID NO: 541 | CATCTAGTTTGCAACGGTCCAGTmU |
| SEQ ID NO: 542 | ACTGGACCGTTGCAAACTAGATGmG |
| SEQ ID NO: 543 | GACTCGCAGCGAGAGAATTTTTTmU |
| SEQ ID NO: 544 | AAAAAATTCTCTCGCTGCGAGTCmC |
| SEQ ID NO: 545 | ATTCACTTTCGGACAAATCGAACmU |
| SEQ ID NO: 546 | GTTCGATTTGTCCGAAAGTGAATmU |
| SEQ ID NO: 547 | AACCATGAGACCTTTTCGCCGCAmG |
| SEQ ID NO: 548 | TGCGGCGAAAAGGTCTCATGGTTmA |
| SEQ ID NO: 549 | GGCCGTTAGATTTTAGTGACGATmG |
| SEQ ID NO: 550 | ATCGTCACTAAAATCTAACGGCCmG |
| SEQ ID NO: 551 | GCAACGTGCCGCATGCATCCATTmC |
| SEQ ID NO: 552 | AATGGATGCATGCGGCACGTTGCmG |
| SEQ ID NO: 553 | ACAGTACATGTAGGAGTACTGTGmC |
| SEQ ID NO: 554 | CACAGTACTCCTACATGTACTGTmG |
| SEQ ID NO: 555 | ACATTCAGTCTCTCTCACTAGTTmG |
| SEQ ID NO: 556 | AACTAGTGAGAGAGACTGAATGTmA |
| SEQ ID NO: 557 | CTACAAAGACATGAGCTGCCGGGmA |
| SEQ ID NO: 558 | CCCGGCAGCTCATGTCTTTGTAGmC |
| SEQ ID NO: 559 | GAGCGAGCGAGCCTGACGGTCTCmA |
| SEQ ID NO: 560 | GAGACCGTCAGGCTCGCTCGCTCmC |
| SEQ ID NO: 561 | ACTCCCAAGCCAATTATTATAAGmA |
| SEQ ID NO: 562 | CTTATAATAATTGGCTTGGGAGTmG |
| SEQ ID NO: 563 | ACTCCAGCTCTTAACCAATCCACmU |
| SEQ ID NO: 564 | GTGGATTGGTTAAGAGCTGGAGTmU |
| SEQ ID NO: 565 | CACCTCCTCTGCTTTGCTCTGCCmA |
| SEQ ID NO: 566 | GGCAGAGCAAAGCAGAGGAGGTGmG |
| SEQ ID NO: 567 | GGGGGCAGAGGAGCTCCCCCTCCmC |
| SEQ ID NO: 568 | GGAGGGGGAGCTCCTCTGCCCCCmC |
| SEQ ID NO: 569 | TCGCCATGTCTAGCAGCGACCCGmG |
| SEQ ID NO: 570 | CGGGTCGCTGCTAGACATGGCGAmG |
| SEQ ID NO: 571 | GCGCGTCGTCGTTCTCGGTTCGCmC |
| SEQ ID NO: 572 | GCGAACCGAGAACGACGACGCGCmG |
| SEQ ID NO: 573 | GGCGACGAGTGGGCCCGGCCCGAmG |
| SEQ ID NO: 574 | TCGGGCCGGGCCCACTCGTCGCCmG |
| SEQ ID NO: 575 | ATCTGCCGTCTCCCGCCCACCAGmC |
| SEQ ID NO: 576 | CTGGTGGGCGGGAGACGGCAGATmG |
| SEQ ID NO: 577 | AGCCGGGCAACCGGAAGCAGCAGmA |
| SEQ ID NO: 578 | CTGCTGCTTCCGGTTGCCCGGCTmA |
| SEQ ID NO: 579 | CCTGCTCCTGCTGGCCGCAGCAGmC |
| SEQ ID NO: 580 | CTGCTGCGGCCAGCAGGAGCAGGmG |
| SEQ ID NO: 581 | CGCCTACTACATCTGCCGGTGGCmG |
| SEQ ID NO: 582 | GCCACCGGCAGATGTAGTAGGCGmU |
| SEQ ID NO: 583 | TCCTCCTTCTTCGCCTCCCCCTCmC |
| SEQ ID NO: 584 | GAGGGGGAGGCGAAGAAGGAGGAmG |

CATS oligonucleotide sequences for silencing
Old Gold gene (mC = 2′-O-methyl cytosine,
mG = 2′-O-methyl guanine, mA = 2′-O-methyl
adenine, mT = 2′-O-methyl thymine, mU =
2′-O-methyl uracil)

| | |
|---|---|
| SEQ ID NO: 585 | GGTTTAAAAAAGATTTCTTTTTTmU |
| SEQ ID NO: 586 | AAAAAAGAAATCTTTTTTAAACCmU |
| SEQ ID NO: 587 | GTAATCGACACACTAATGCAAAGmA |
| SEQ ID NO: 588 | CTTTGCATTAGTGTGTCGATTACmU |
| SEQ ID NO: 589 | AACATCTTGGACCTAAATAATTGmU |
| SEQ ID NO: 590 | CAATTATTTAGGTCCAAGATGTTmU |
| SEQ ID NO: 591 | CTTTCCATTTTCATCTTTAAATAmU |
| SEQ ID NO: 592 | TATTTAAAGATGAAAATGGAAAGmG |
| SEQ ID NO: 593 | ACAATTTTTTTTTGGGCTAAAATmG |
| SEQ ID NO: 594 | ATTTTAGCCCAAAAAAAAATTGTmG |
| SEQ ID NO: 595 | TGGTGGAGTTATGACCACATATTmG |
| SEQ ID NO: 596 | AATATGTGGTCATAACTCCACCAmU |
| SEQ ID NO: 597 | AGTGCTCAAAAGGAGAGTCTACTmG |
| SEQ ID NO: 598 | AGTAGACTCTCCTTTTGAGCACTmU |
| SEQ ID NO: 599 | CCACCACAAGTACTATGCAACAAmA |
| SEQ ID NO: 600 | TTGTTGCATAGTACTTGTGGTGGmA |
| SEQ ID NO: 601 | AAGAAAATGGAAACTTTTCTCTCmU |
| SEQ ID NO: 602 | GAGAGAAAAGTTTCCATTTTCTTmG |
| SEQ ID NO: 603 | CACTAGCTGTTTACATGGTGAGCmA |
| SEQ ID NO: 604 | GCTCACCATGTAAACAGCTAGTGmA |
| SEQ ID NO: 605 | AGAAATACTTAGTATATATCTATmA |
| SEQ ID NO: 606 | ATAGATATATACTAAGTATTTCTmA |
| SEQ ID NO: 607 | ACTTTTCATTCTGTAATTCTTTAmA |
| SEQ ID NO: 608 | TAAAGAATTACAGAATGAAAAGTmG |
| SEQ ID NO: 609 | CTGTTTAAAGCTTGATTTTTTTTmA |
| SEQ ID NO: 610 | AAAAAAAATCAAGCTTTAAACAGmU |
| SEQ ID NO: 611 | ATGTTCTGCTTCATTTGTGTTGAmA |
| SEQ ID NO: 612 | TCAACACAAATGAAGCAGAACATmG |
| SEQ ID NO: 613 | ATTGGGGAACTTTCTTGAATCCAmG |
| SEQ ID NO: 614 | TGGATTCAAGAAAGTTCCCCAATmU |
| SEQ ID NO: 615 | CATTTGAAGTTTTCTTGAAACAAmA |
| SEQ ID NO: 616 | TTGTTTCAAGAAAACTTCAAATGmG |
| SEQ ID NO: 617 | CATTACCCTGTTGGAAAAAGATGmG |
| SEQ ID NO: 618 | CATCTTTTTCCAACAGGGTAATGmA |
| SEQ ID NO: 619 | TACTTTGTTGAAAACCCCAAATAmA |
| SEQ ID NO: 620 | TATTTGGGGTTTTCAACAAAGTAmU |
| SEQ ID NO: 621 | CTTGAATTTCTGAACCCACATCAmU |
| SEQ ID NO: 622 | TGATGTGGGTTCAGAAATTCAAGmG |
| SEQ ID NO: 623 | GTTTTGCTGTTAAAGCTAGTACCmU |
| SEQ ID NO: 624 | GGTACTAGCTTTAACAGCAAAACmC |
| SEQ ID NO: 625 | GGTTCTAGGAAGTTTTGTGAAACmU |
| SEQ ID NO: 626 | GTTTCACAAAACTTCCTAGAACCmA |
| SEQ ID NO: 627 | TGGGTAGAAGTGTTTGTGTTAAGmG |
| SEQ ID NO: 628 | CTTAACACAAACACTTCTACCCAmA |
| SEQ ID NO: 629 | TAGTAGTAGTGCTCTTTTAGAGCmU |
| SEQ ID NO: 630 | GCTCTAAAAGAGCACTACTACTAmC |
| SEQ ID NO: 631 | GTACCTGAGACCAAAAAGGAGAAmU |
| SEQ ID NO: 632 | TTCTCCTTTTTGGTCTCAGGTACmA |
| SEQ ID NO: 633 | TTGATTTTGAGCTTCCTATGTATmG |
| SEQ ID NO: 634 | ATACATAGGAAGCTCAAAATCAAmG |
| SEQ ID NO: 635 | CTTGCTGTGGTTGGTGGTGGCCCmU |
| SEQ ID NO: 636 | GGGCCACCACCAACCACAGCAAGmA |
| SEQ ID NO: 637 | CAGGACTTGCTGTTGCACAGCAAmG |
| SEQ ID NO: 638 | TTGCTGTGCAACAGCAAGTCCTGmC |
| SEQ ID NO: 639 | TTCTGAAGCAGGACTCTCTGTTTmG |
| SEQ ID NO: 640 | AAACAGAGAGTCCTGCTTCAGAAmA |
| SEQ ID NO: 641 | TCAATTGATCCGAATCCTAAATTmG |
| SEQ ID NO: 642 | AATTTAGGATTCGGATCAATTGAmA |
| SEQ ID NO: 643 | TATGGCCTAATAACTATGGTGTTmU |
| SEQ ID NO: 644 | AACACCATAGTTATTAGGCCATAmU |
| SEQ ID NO: 645 | TTGTTAGATTGTCTAGATGCTACmC |
| SEQ ID NO: 646 | GTAGCATCTAGACAATCTAACAAmG |
| SEQ ID NO: 647 | GGTCTGGTGCAGCAGTGTACATTmG |
| SEQ ID NO: 648 | AATGTACACTGCTGCACCAGACCmA |
| SEQ ID NO: 649 | TGATAATACGGCTAAAGATCTTCmA |
| SEQ ID NO: 650 | GAAGATCTTTAGCCGTATTATCAmU |
| SEQ ID NO: 651 | AGACCTTATGGAAGGGTTAACCGmG |
| SEQ ID NO: 652 | CGGTTAACCCTTCCATAAGGTCTmA |
| SEQ ID NO: 653 | AACAGCTGAAATCGAAAATGATGmC |
| SEQ ID NO: 654 | CATCATTTTCGATTTCAGCTGTTmU |
| SEQ ID NO: 655 | TTCCACCAAGCCAAAGTTATAAAmG |
| SEQ ID NO: 656 | TTTATAACTTTGGCTTGGTGGAAmU |
| SEQ ID NO: 657 | TGATTCATGAGGAATCGAAATCCmA |
| SEQ ID NO: 658 | GGATTTCGATTCCTCATGAATCAmC |
| SEQ ID NO: 659 | GTTGATATGCAATGATGGTATTAmC |
| SEQ ID NO: 660 | TAATACCATCATTGCATATCAACmA |
| SEQ ID NO: 661 | ATTCAGGCAACGGTGGTGCTCGAmU |
| SEQ ID NO: 662 | TCGAGCACCACCGTTGCCTGAATmA |
| SEQ ID NO: 663 | CAACTGGCTTCTCTAGATCTCTTmG |
| SEQ ID NO: 664 | AAGAGATCTAGAGAAGCCAGTTGmC |

What is claimed is:

1. A method of inducing an epigenetic modification in a plant organism, comprising applying a formulation to an agricultural object, the formulation comprising an excipient and a double stranded artificial nucleic acid construct comprising (a) a first strand and a second strand each having a length of 20 to 30 nucleotides or nucleosides or a combination thereof; and (b) a terminal overhang at one or both of the 3′ ends of the first strand and/or second strand;
wherein: one or both of the 3′ terminal end overhangs comprise a ribose unit having a methoxy group (—O-Me) at a 2′ position;
wherein the nucleic acid construct does not comprise a regulatory element,
wherein at least 80% of the sugars in the double stranded artificial nucleic acid construct comprises a deoxyribose,
wherein the agricultural object is a seed or a plant, or a combination thereof,
wherein the applying causes methylation of at least one base of a nucleotide or nucleoside in a nucleic acid sequence of a gene of the agricultural object, and
wherein the applying causes methylation via an interaction between the artificial nucleic acid construct and an endogenous DNA methyltransferase.

2. The method of claim 1, wherein one strand of the nucleic acid construct comprises at least 80% sequence identity to a portion of a transcription regulatory region of the gene.

3. The method of claim 1, wherein one strand of the nucleic acid construct comprises at least 100% sequence identity to a portion of a transcription regulatory region of the gene.

4. The method of claim 1, wherein the transcription regulatory region is a transcription start site, a TATA box, or an upstream activating sequence.

5. The method of claim 1, wherein every sugar in the double stranded artificial nucleic construct is a deoxyribose except for the terminal 3′ sugar of the first strand and the second strand.

6. The method of claim 1, wherein the at least one base is a cytosine.

7. The method of claim 1, wherein both of the 3′ ends of the first strand and second strand comprise a terminal overhang and each terminal end overhang is one nucleotide long.

8. The method of claim 1, wherein the agricultural object is a plant and the formulation is applied to a leaf, a stalk, a root, a flower,, or any combination thereof.

9. The method of claim 1, wherein the agricultural object comprises a seed.

10. The method of claim 1, wherein the method results in at least partial silencing of the gene in the agricultural object for at least one reproduction cycle of the agricultural object.

11. The method of claim 1, wherein the plant organism is soybean, corn, rice, tomato, alfalfa, wheat, potato, or pea.

12. The method of claim 1, wherein the formulation is a liquid.

13. The method of claim 1, wherein the excipient is water.

14. The method of claim 1, wherein the first strand and the second strand each have a length of 24 nucleotides.
